(19) United States
(12) Patent Application Publication
(10) Pub. No.: US 2020/0235306 A1
(43) Pub. Date: Jul. 23, 2020

(54) ORGANIC COMPOUND AND ORGANIC ELECTROLUMINESCENCE DEVICE USING THE SAME

(71) Applicants: FENG-WEN YEN, TAIPEI (TW); WAN-SHIUAN WU, TAIPEI (TW)

(72) Inventors: FENG-WEN YEN, TAIPEI (TW); WAN-SHIUAN WU, TAIPEI (TW)

(73) Assignee: LUMINESCENCE TECHNOLOGY CORPORATION, HSINCHU (TW)

(21) Appl. No.: 16/252,688

(22) Filed: Jan. 20, 2019

(51) Int. Cl. H01L 51/00; C07D 405/10; C07D 405/14; C07D 471/04; C07D 491/048; C07D 495/04; C09K 11/06; C09K 11/02

(52) U.S. Cl. CPC H01L 51/0067; C07D 405/10; H01L 51/0073; C07D 405/14; C07D 471/04; H01L 51/5012; C07D 491/048; C07D 495/04; C09K 11/06; C09K 11/025; C09K 2211/1018; H01L 51/0072

(57) ABSTRACT

The present invention discloses an organic compound resented by the following formula (1) and an organic electroluminescence device using the organic compound. The organic compound may be for lowering a driving voltage or power consumption or increasing a current efficiency of half-life of the organic electroluminescence device.

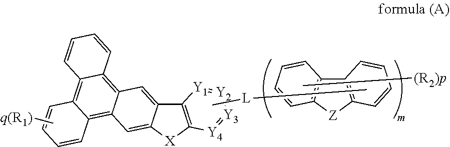

The same definition as described in the present invention.

### FIELD OF INVENTION

The present invention relates generally to an organic compound and, more specifically, to an organic electroluminescence device using the organic compound.

### BACKGROUND OF THE INVENTION

An organic electroluminescence (organic EL) device is an organic light-emitting diode (OLED) in which the light emitting layer is a film made from organic compounds, which emits light in response to an electric current. The light emitting layer containing the organic compound is sandwiched between two electrodes. The organic EL device is applied to flat panel displays due to its high illumination, low weight, ultra-thin profile, self-illumination without back light, low power consumption, wide viewing angle, high contrast, simple fabrication methods and rapid response time.

Typically, organic EL device is composed of organic material layers sandwiched between two electrodes. The organic material layers include, e.g., hole injection layer (HIL), hole transporting layer (HTL), emitting layer (EML), electron transporting layer (ETL), and electron injection layer (EIL). The basic mechanism of organic EL involves the injection, transport, and recombination of carriers as well as exciton formation for emitting light. When an external voltage is applied across the organic EL device, electrons and holes are injected from the cathode and the anode, respectively. Electrons will be injected from the cathode into a LUMO (lowest unoccupied molecular orbital) and holes will be injected from the anode into a HOMO (highest occupied molecular orbital). Subsequently, the electrons recombine with holes in the light emitting layer to form excitons, which then deactivate to emit light. When luminescent molecules absorb energy to achieve an excited state, the exciton may either be in a singlet state or a triplet state, depending on how the spins of the electrons and holes have been combined. It is well known that the excitons formed under electrical excitation typically include 25% singlet excitons and 75% triplet excitons. In the fluorescence materials, however, the electrically generated energy in the 75% triplet excitons will be dissipated as heat for decay from the triplet state is spin forbidden. Therefore, a fluorescent electroluminescence device has only 25% internal quantum efficiency, which leads to the theoretically highest external quantum efficiency (EQE) of only 5% due to only ˜20% of the light out-coupling efficiency of the device. In contrast to fluorescent electroluminescence devices, phosphorescent organic EL devices make use of spin-orbit interactions to facilitate intersystem crossing between singlet and triplet states, thus obtaining emission from both singlet and triplet states and the internal quantum efficiency of electroluminescence devices from 25% to 100%.

However, there is still a need for improvement in the case of use of those organic materials in an organic EL device of some prior art displays, for example, in relation to the half-lift time, current efficiency or driving voltage of the organic EL device.

### SUMMARY OF THE INVENTION

Accordingly, an object of the invention is to provide an organic compound and an organic EL device using the same, which can exhibit improved luminance, current efficiency, or half-lift time.

Another object of the invention is to provide an organic compound and an organic EL device using the same, which may lower a driving voltage or increasing a current efficiency or half-life time for the organic EL device.

Still another object of the present invention is to provide an organic compound, which can be used as a hole blocking material (HBM), electron transport material (ETM) or phosphorescent host in an organic EL device to improve driving voltage, current efficiency or half-life time.

According to the invention, an organic compound which may be used in organic EL devices is disclosed. The organic compound may be represented by the following formula (A):

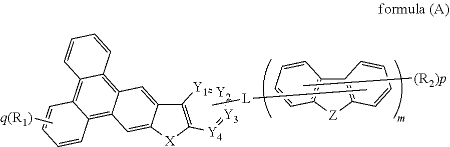

wherein X may be a divalent bridge selected from the group consisting of O and S. The symbol m may represent 0, 1 or 2. L may represent a single bond, a substituted or unsubstituted aryl group having 6 to 40 ring carbon atoms (e.g., 6), or a substituted or unsubstituted heteroaryl group having 3 to 40 (e.g., 6, 12, 14, 18) ring carbon atoms, if m represents 1 or 2. L may represent a substituted or unsubstituted heteroaryl group having 3 to 40 (e.g., 12, 16, 18, 20, 21, 22, 23, 24, 26, 28, 30, 31, 32, 33, 34, 35, 36, 37) ring carbon atoms, if m represents 0. Z may represent O, S or NR₃. Y₁ to Y₄ may independently represent nitrogen atom or CR₄. R₃ and R₄ may independently represent a hydrogen atom, a substituent, or a bond to L. p may represent an integer of 0 to 7 (e.g., 1 or 2), q may represent an integer of 0 to 10 (e.g., 1). R₁ and R₂ may be independently selected from the group consisting of a hydrogen atom, a halide, alkyl group having 1 to 20 (e.g., 1, 4 or 6) carbon atoms, a substituted or unsubstituted aryl group having 6 to 30 carbon atoms (e.g., 6, 10, 14, 16, 18, 24), a substituted or unsubstituted aralkyl group having 7 to 30 carbon atoms, a substituted or unsubstituted heteroaryl group having 3 to 30 carbon atoms.

L may also represent a substituted phenyl group, a substituted triazinyl group, a substituted diazinyl group, a substituted pyridinyl group, or a substituted derivative of diazine group.

R₂ and R₃ may also represent a substituted or unsubstituted phenyl group, a substituted or unsubstituted biphenyl group, a substituted or unsubstituted naphthyl group, a substituted or unsubstituted anthracenyl group, a substituted or unsubstituted phenanthrenyl group, a substituted or unsubstituted phenanthrolinyl group, a substituted or unsubstituted triphenylenyl group, a substituted or unsubstituted pyrenyl group, a substituted or unsubstituted fluorenyl group, a substituted or unsubstituted dibenzofuranyl group, a substituted or unsubstituted benzonaphthofuranyl group, a substituted or unsubstituted dibenzothiophenyl group, a substituted or unsubstituted carbazolyl group, a substituted or unsubstituted biscarbazolyl group, a substituted or unsubstituted benzocarbazolyl group, a substituted or unsubstituted phenothiazinyl group, a substituted or unsubstituted phenoxazinyl group, a substituted or unsubstituted dimethylcarbazinyl group, a substituted or unsubstituted benzimidazole group, a substituted or unsubstituted triazinyl group, a substituted or unsubstituted diazinyl group, or a substituted or unsubstituted pyridinyl group.

The present invention further discloses an organic electroluminescence device. The organic electroluminescence (EL) device comprises a pair of electrodes having a cathode and an anode. The organic EL device may comprise, a light emitting layer and one or more layers of organic thin film layers between the pair of electrodes. The light emitting layer and/or the one or more organic thin film layers comprise an organic compound of formula (A). The electron transport material may be doped with about 40% LiQ. The electron transport layer may have a thickness of about 35 nm.

The light emitting layer comprising the organic compound of formula (A) may be a host material. The one or more layers of organic thin film layers comprises an electron transport layer having an organic material of formula (A). The one or more layers of organic thin film layers may comprise a hole blocking layer having an organic material of formula (A). The light emitting layer comprising the organic compound of formula (A) may be a fluorescent emitter. The light emitting layer may emit fluorescent blue lights. The device may be an organic light emitting device. The device may be a lighting panel or a backlight panel.

An organic EL device of the present invention comprises an organic compound of formula (A) as an electron transport material to collocate with, for example, a fluorescent host material H2 to emit a blue light, thereby lowing a driving voltage to about 4.3-5.9V, increasing a current efficiency to about 5.2-6.3 cd/A, or increasing a half-life time to about 320-580 hours.

To emit a red light, an organic EL device of the present invention comprises an organic compound of formula (A) as a phosphorescent host material to collocate with, for example, a hole blocking material HB3, thereby lowing a driving voltage to about 4.3-4.6V, increasing a current efficiency to about 19-22 cd/A, or increasing a half-life time to about 460-540 hours. The organic compound may be, for example, selected from but not limited to the group consisting of compounds A7, A4, A11, A139, A13, A160, A12, A80, A168, A129 and the combination thereof, as shown in the description of the preferred embodiments.

To emit a red light, an organic EL device of the present invention comprises an organic compound of formula (A) as a hole blocking material to collocate with, for example, phosphorescent host material A7 and A139, thereby lowing a driving voltage to about 4.3-4.6V, increasing a current efficiency to about 21-24 cd/A, or increasing a half-life time to about 470-500 hours. The organic compound may be, for example, selected from but not limited to the group consisting of compounds A4, A7, A27, A29 and the combination thereof, as shown in the description of the preferred embodiments.

To emit a red light, an organic EL device of the present invention comprises an organic compound of formula (A) as a phosphorescent host material to collocate with, for example, a hole blocking material A139, thereby lowing a driving voltage to about 4.2-4.6V, increasing a current efficiency to about 19-25 cd/A, or increasing a half-life time to about 490-630 hours. The organic compound may be, for example, selected from but not limited to the group consisting of compounds A139, A167, A110, A143, A182, A171, A35, A143 and the combination thereof, as shown in the description of the preferred embodiments.

### BRIEF DESCRIPTION OF THE DRAWINGS

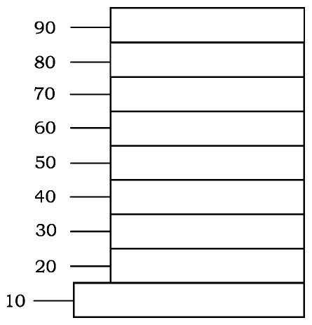

The FIG. 1 is a schematic view showing an organic EL device according to an embodiment of the present invention.

### DESCRIPTION OF THE PREFERRED EMBODIMENTS

What probed into the invention is the organic compound and organic EL device using the organic compound. Detailed descriptions of the production, structure and elements will be provided as follows such that the invention can be fully understand. Obviously, the application of the invention is not confined to specific details familiar to those skilled in the art. On the other hand, the common elements and procedures that are known to everyone are not described in detail to avoid unnecessary limits of the invention. Some preferred embodiments of the present invention will now be described in greater detail as follows. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

In one embodiment of the present invention, an organic compound can be used as a hole blocking material (HBM), an electron transport material (ETM) or phosphorescent host material to lower driving voltage, increase current efficiency or half-life time for organic EL devices is disclosed. The organic compound may be represented by the following formula (A):

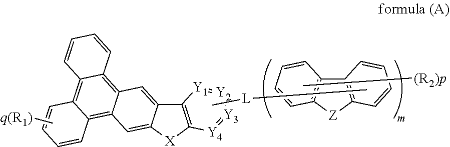

wherein X may be independently a divalent bridge selected from the group consisting of O and S. The symbol m may represent 01, or 2. L may represent a single bond, a substituted or unsubstituted aryl group having 6 to 40 ring carbon atoms, or a substituted or unsubstituted heteroaryl group having 3 to 40 ring carbon atoms, if m represents 1 or 2. L may represent a substituted or unsubstituted heteroaryl group having 3 to 40 ring carbon atoms, if m represents 0. Z may represent O, S or NR₃. Y₁ to Y₄ may independently represent a nitrogen atom or CR₄. R₃ and R₄ may independently represent a hydrogen atom, a substituent, or a bond to L. The symbol p may represent an integer of 0, 1, 2, 3, 4, 5, 6 or 7. The symbol q may represent an integer of 0, 1, 2, 3, 4, 5, 6, 7, 8, 9 or 10. R₁ and R₂ may be independently selected from the group consisting of a hydrogen atom, a halide, an alkyl group having 1 to 20 carbon atoms, a substituted or unsubstituted aryl group having 6 to 30 carbon atoms, a substituted or unsubstituted aralkyl group having 7 to 30 carbon atoms, or a substituted or unsubstituted heteroaryl group having 3 to 30 carbon atoms.

In some embodiments, L may be selected from the group consisting of:

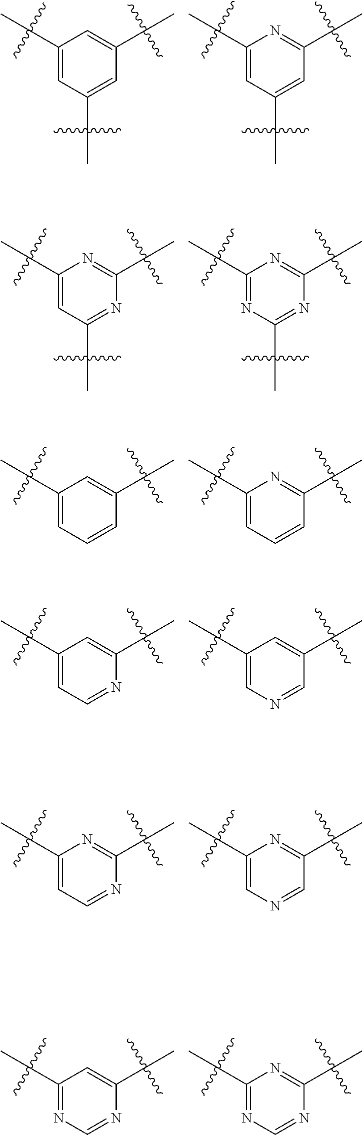

if m represents 1 or 2.

L may be selected from the group consisting of:

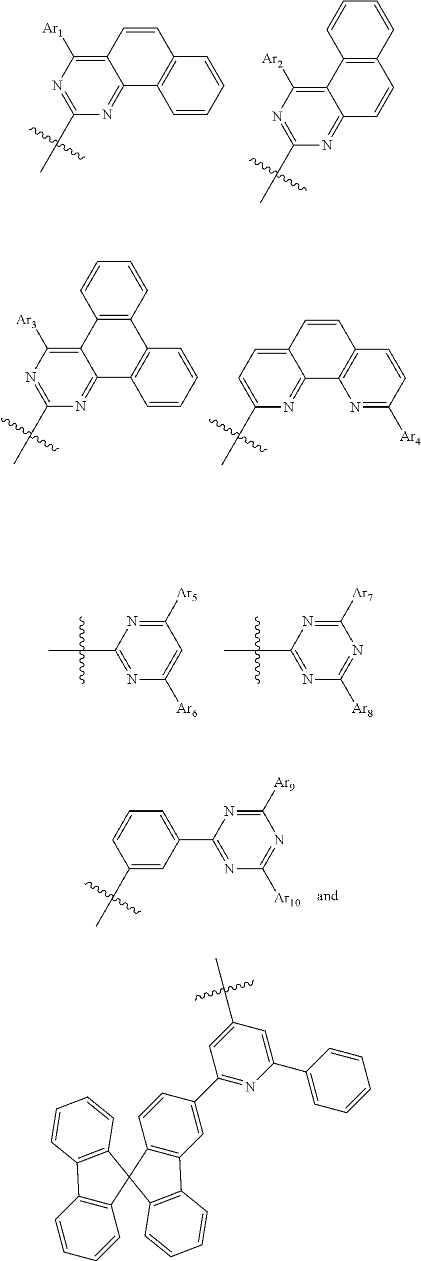

if m represents 0, and wherein Ar₁ to Ar₄ independently represent a hydrogen atom, a substituted or unsubstituted aryl group having 6 to 30 ring carbon atoms, and wherein Ar₈ to Ar₁₀ independently represent a hydrogen atom, a substituted or unsubstituted aryl group having 6 to 30 ring carbon atoms, or a substituted or unsubstituted heteroaryl group having 3 to 30 ring carbon atoms. L may also be selected from the group consisting of:

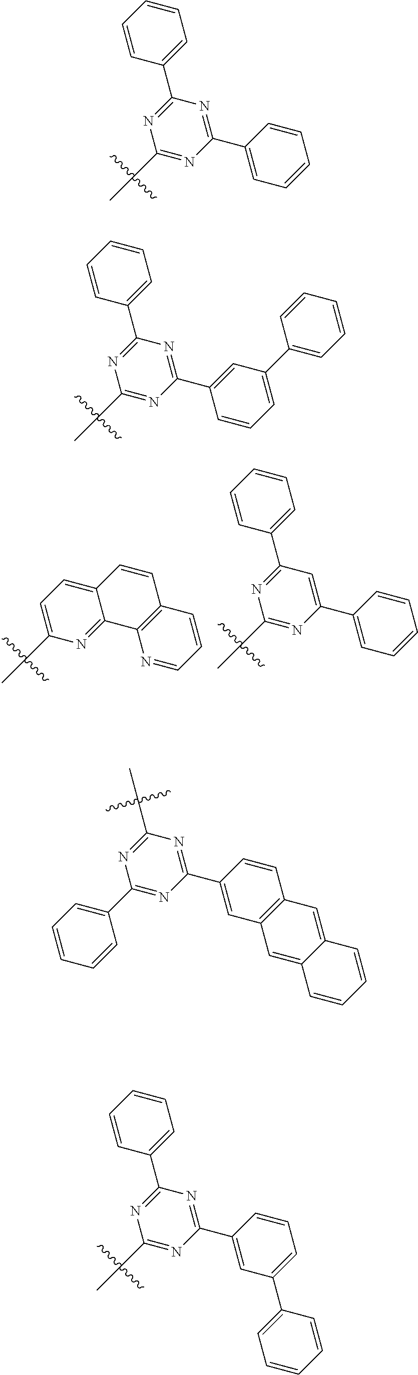

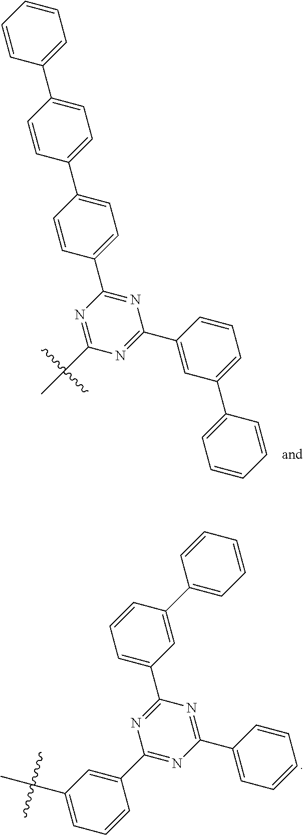 [SMILES: *c1nc(-c2ccc(-c3ccc(-c4ccccc4)cc3)cc2)nc(-c2cccc(-c3ccccc3)c2)n1 ; *c1cccc(-c2nc(-c3ccccc3)nc(-c3cccc(-c4ccccc4)c3)n2)c1]

In some embodiments, the organic compound may be one of the following compounds:

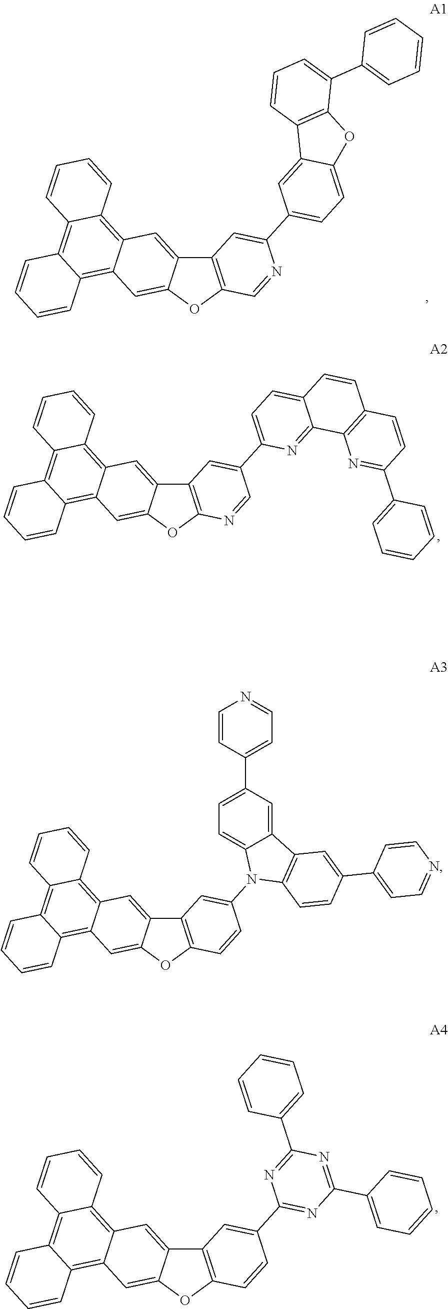 [SMILES: c1ccc(-c2cccc3c2oc2ccc(-c4cc5c(cn4)oc4cc6c7ccccc7c7ccccc7c6cc45)cc23)cc1 ; c1ccc(-c2ccc3ccc4ccc(-c5cnc6oc7cc8c9ccccc9c9ccccc9c8cc7c6c5)nc4c3n2)cc1 ; c1ccc2c(c1)c1ccccc1c1cc3c(cc21)oc1ccc(-n2c4ccc(-c5ccncc5)cc4c4cc(-c5ccncc5)ccc42)cc13 ; c1ccc(-c2nc(-c3ccccc3)nc(-c3ccc4oc5cc6c7ccccc7c7ccccc7c6cc5c4c3)n2)cc1]

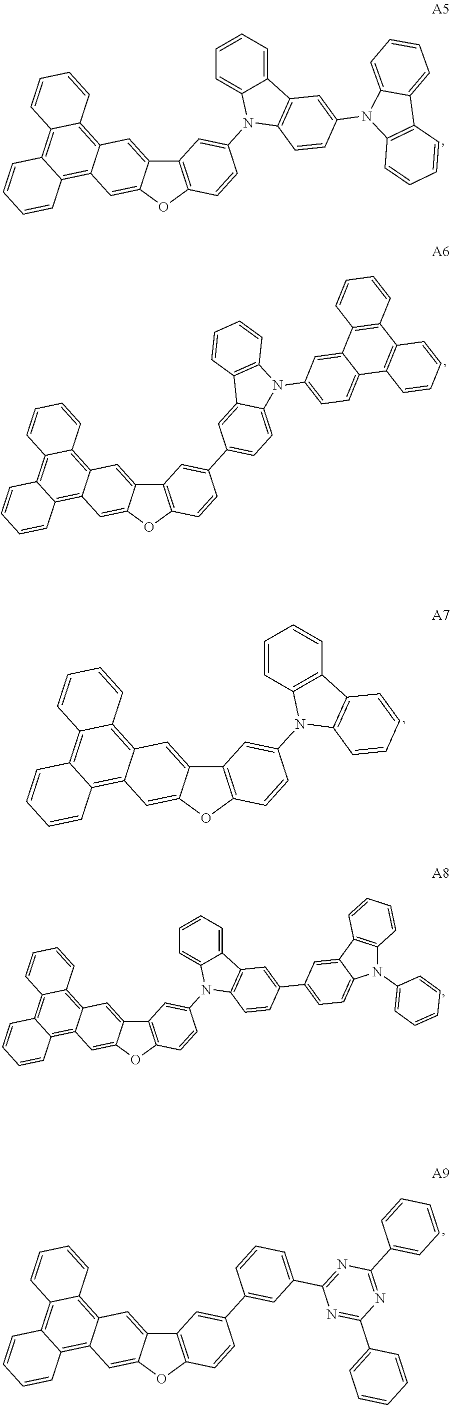 [SMILES: c1ccc2c(c1)c1ccccc1c1cc3c(cc21)oc1ccc(-n2c4ccccc4c4cc(-n5c6ccccc6c6ccccc65)ccc42)cc13 ; c1ccc2c(c1)c1ccccc1c1cc(-n3c4ccccc4c4cc(-c5ccc6oc7cc8c9ccccc9c9ccccc9c8cc7c6c5)ccc43)ccc21 ; c1ccc2c(c1)c1ccccc1c1cc3c(cc21)oc1ccc(-n2c4ccccc4c4ccccc42)cc13 ; c1ccc(-n2c3ccccc3c3cc(-c4ccc5c(c4)c4ccccc4n5-c4ccc5oc6cc7c8ccccc8c8ccccc8c7cc6c5c4)ccc32)cc1 ; c1ccc(-c2nc(-c3ccccc3)nc(-c3cccc(-c4ccc5oc6cc7c8ccccc8c8ccccc8c7cc6c5c4)c3)n2)cc1]

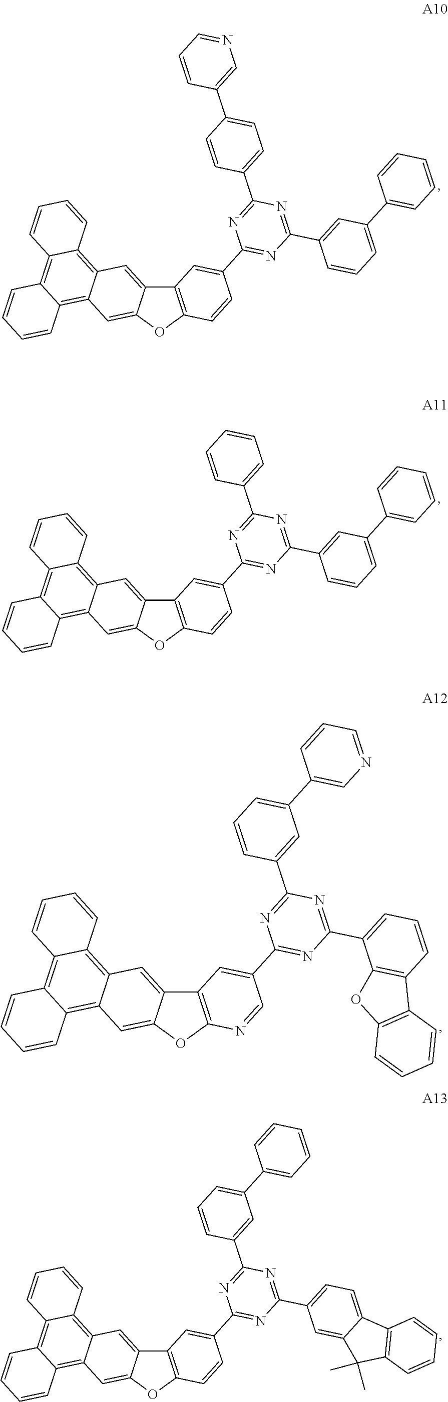 [SMILES: c1ccc(-c2cccc(-c3nc(-c4ccc(-c5cccnc5)cc4)nc(-c4ccc5oc6cc7c8ccccc8c8ccccc8c7cc6c5c4)n3)c2)cc1 ; c1ccc(-c2cccc(-c3nc(-c4ccccc4)nc(-c4ccc5oc6cc7c8ccccc8c8ccccc8c7cc6c5c4)n3)c2)cc1 ; c1cncc(-c2cccc(-c3nc(-c4cnc5oc6cc7c8ccccc8c8ccccc8c7cc6c5c4)nc(-c4cccc5c4oc4ccccc45)n3)c2)c1 ; CC1(C)c2ccccc2-c2ccc(-c3nc(-c4cccc(-c5ccccc5)c4)nc(-c4ccc5oc6cc7c8ccccc8c8ccccc8c7cc6c5c4)n3)cc21]

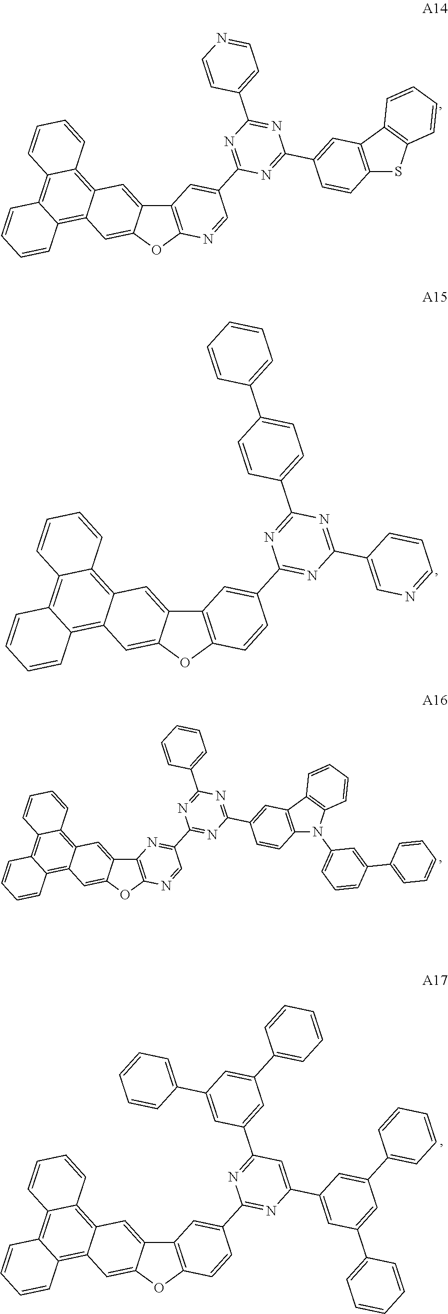 [SMILES: c1ccc2c(c1)sc1ccc(-c3nc(-c4ccncc4)nc(-c4cnc5oc6cc7c8ccccc8c8ccccc8c7cc6c5c4)n3)cc12 ; c1ccc(-c2ccc(-c3nc(-c4cccnc4)nc(-c4ccc5oc6cc7c8ccccc8c8ccccc8c7cc6c5c4)n3)cc2)cc1 ; c1ccc(-c2cccc(-n3c4ccccc4c4cc(-c5nc(-c6ccccc6)nc(-c6cnc7oc8cc9c%10ccccc%10c%10ccccc%10c9cc8c7n6)n5)ccc43)c2)cc1 ; c1ccc(-c2cc(-c3ccccc3)cc(-c3cc(-c4cc(-c5ccccc5)cc(-c5ccccc5)c4)nc(-c4ccc5oc6cc7c8ccccc8c8ccccc8c7cc6c5c4)n3)c2)cc1]

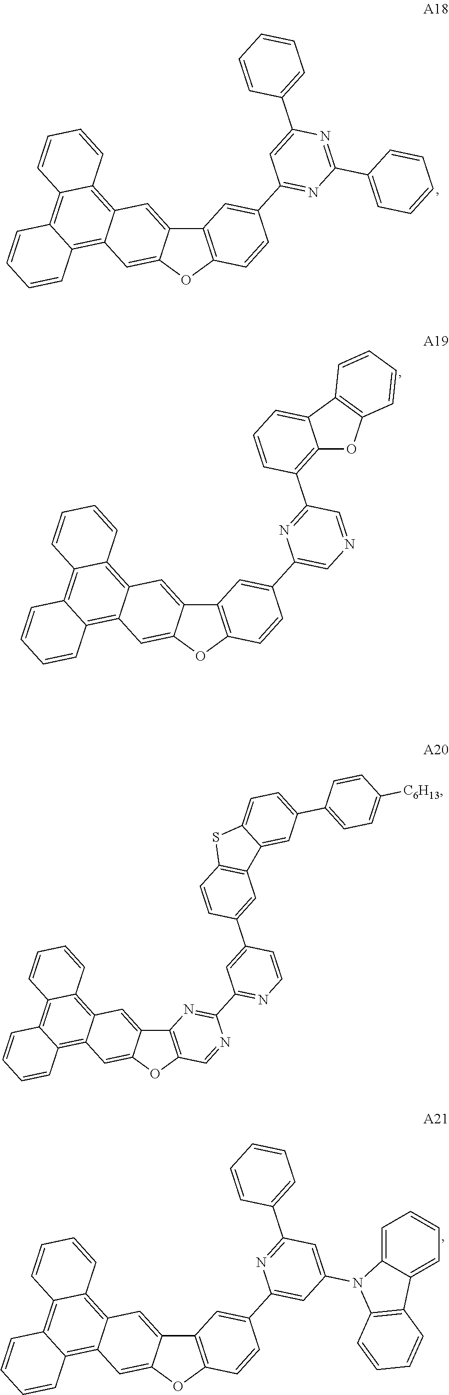

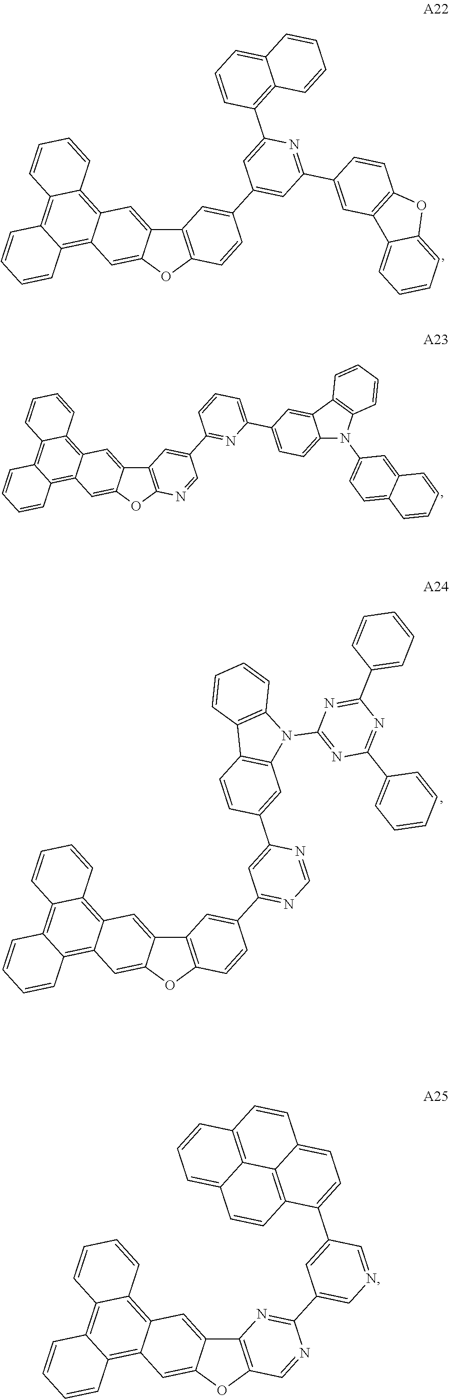 [SMILES: c1ccc2c(-c3cc(-c4ccc5oc6cc7c8ccccc8c8ccccc8c7cc6c5c4)cc(-c4ccc5oc6ccccc6c5c4)n3)cccc2c1 ; c1cc(-c2cnc3oc4cc5c6ccccc6c6ccccc6c5cc4c3c2)nc(-c2ccc3c(c2)c2ccccc2n3-c2ccc3ccccc3c2)c1 ; c1ccc(-c2nc(-c3ccccc3)nc(-n3c4ccccc4c4ccc(-c5cc(-c6ccc7oc8cc9c%10ccccc%10c%10ccccc%10c9cc8c7c6)ncn5)cc43)n2)cc1 ; c1cc2ccc3ccc(-c4cncc(-c5ncc6oc7cc8c9ccccc9c9ccccc9c8cc7c6n5)c4)c4ccc(c1)c2c34]

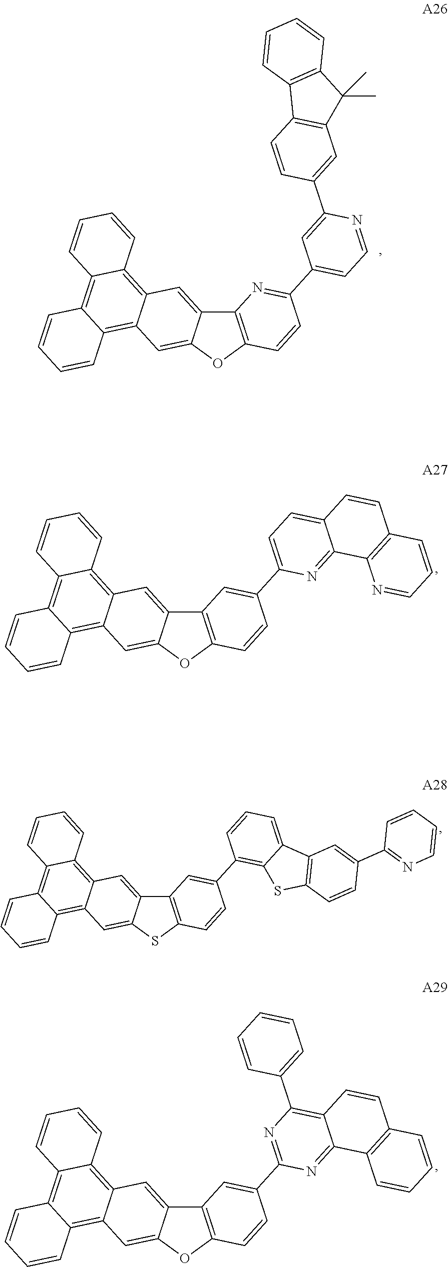 [SMILES: CC1(C)c2ccccc2-c2ccc(-c3cc(-c4ccc5oc6cc7c8ccccc8c8ccccc8c7cc6c5n4)ccn3)cc21 ; c1cnc2c(c1)ccc1ccc(-c3ccc4oc5cc6c7ccccc7c7ccccc7c6cc5c4c3)nc12 ; c1ccc(-c2ccc3sc4c(-c5ccc6sc7cc8c9ccccc9c9ccccc9c8cc7c6c5)cccc4c3c2)nc1 ; c1ccc(-c2nc(-c3ccc4oc5cc6c7ccccc7c7ccccc7c6cc5c4c3)nc3c2ccc2ccccc23)cc1]

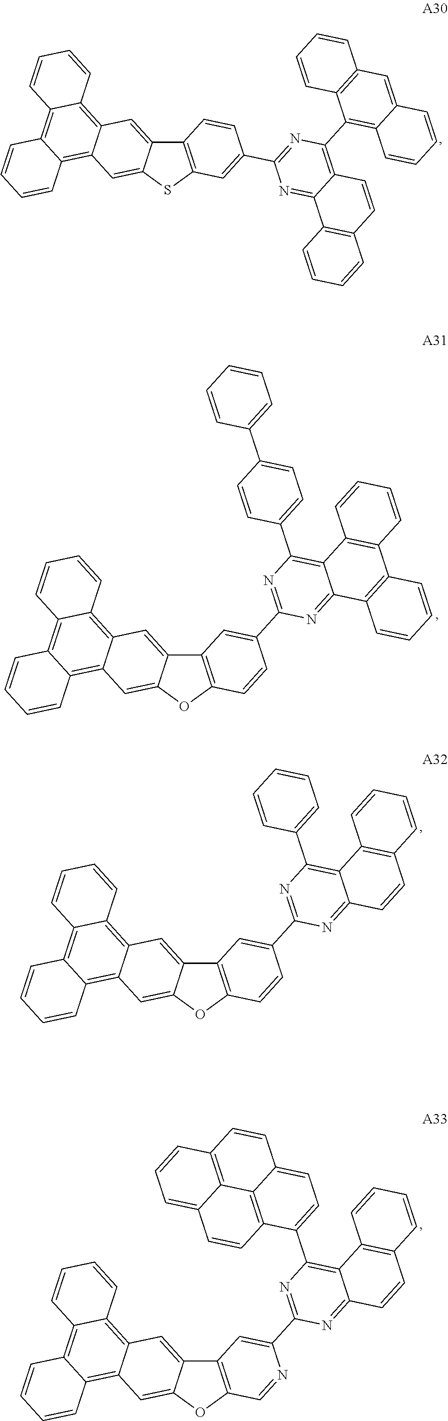 [SMILES: c1ccc2c(-c3nc(-c4ccc5c(c4)sc4cc6c7ccccc7c7ccccc7c6cc45)nc4c3ccc3ccccc34)c3ccccc3cc2c1 ; c1ccc(-c2ccc(-c3nc(-c4ccc5oc6cc7c8ccccc8c8ccccc8c7cc6c5c4)nc4c5ccccc5c5ccccc5c34)cc2)cc1 ; c1ccc(-c2nc(-c3ccc4oc5cc6c7ccccc7c7ccccc7c6cc5c4c3)nc3ccc4ccccc4c23)cc1 ; c1ccc2c(c1)ccc1nc(-c3cc4c(cn3)oc3cc5c6ccccc6c6ccccc6c5cc34)nc(-c3ccc4ccc5cccc6ccc3c4c56)c12]

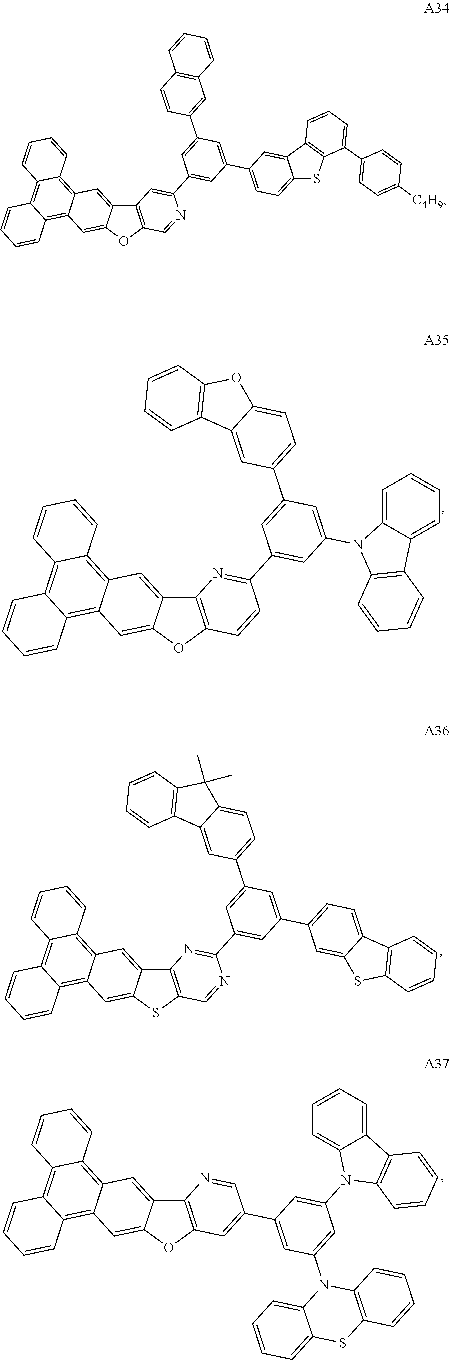

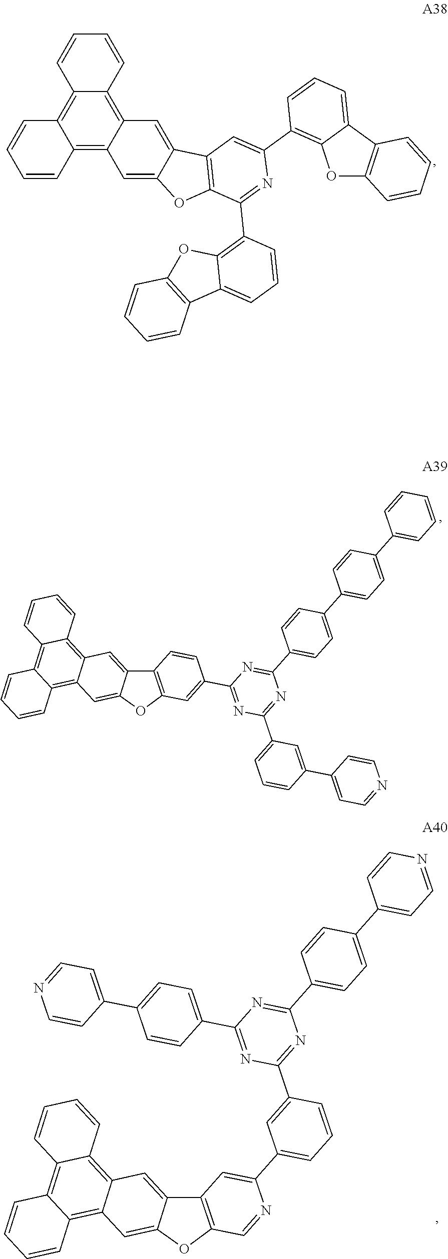 [SMILES: c1ccc2c(c1)oc1c(-c3cc4c(oc5cc6c7ccccc7c7ccccc7c6cc54)c(-c4cccc5c4oc4ccccc45)n3)cccc12 ; c1ccc(-c2ccc(-c3ccc(-c4nc(-c5cccc(-c6ccncc6)c5)nc(-c5ccc6c(c5)oc5cc7c8ccccc8c8ccccc8c7cc56)n4)cc3)cc2)cc1 ; c1cc(-c2cc3c(cn2)oc2cc4c5ccccc5c5ccccc5c4cc23)cc(-c2nc(-c3ccc(-c4ccncc4)cc3)nc(-c3ccc(-c4ccncc4)cc3)n2)c1]

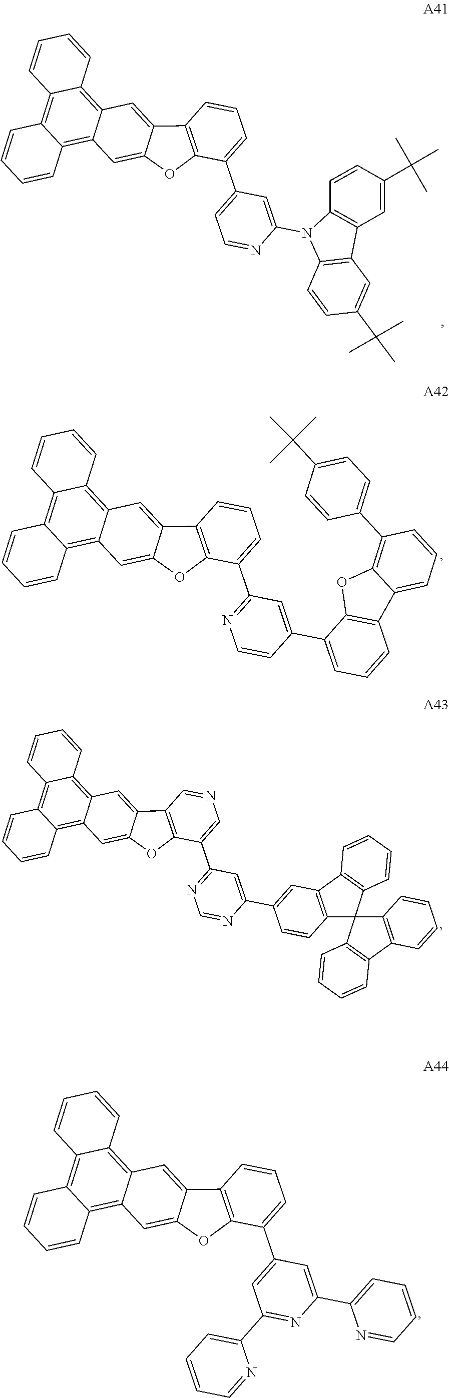 [SMILES: CC(C)(C)c1ccc2c(c1)c1cc(C(C)(C)C)ccc1n2-c1cc(-c2cccc3c2oc2cc4c5ccccc5c5ccccc5c4cc23)ccn1 ; CC(C)(C)c1ccc(-c2cccc3c2oc2c(-c4ccnc(-c5cccc6c5oc5cc7c8ccccc8c8ccccc8c7cc56)c4)cccc23)cc1 ; c1ccc2c(c1)-c1ccccc1C21c2ccccc2-c2cc(-c3cc(-c4cncc5c4oc4cc6c7ccccc7c7ccccc7c6cc45)ncn3)ccc21 ; c1ccc(-c2cc(-c3cccc4c3oc3cc5c6ccccc6c6ccccc6c5cc34)cc(-c3ccccn3)n2)nc1]

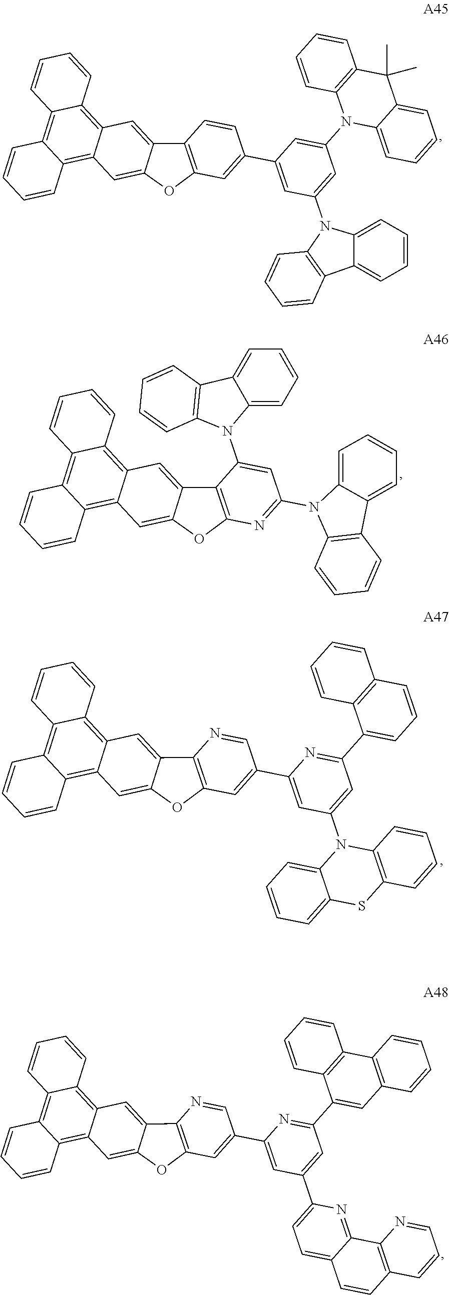

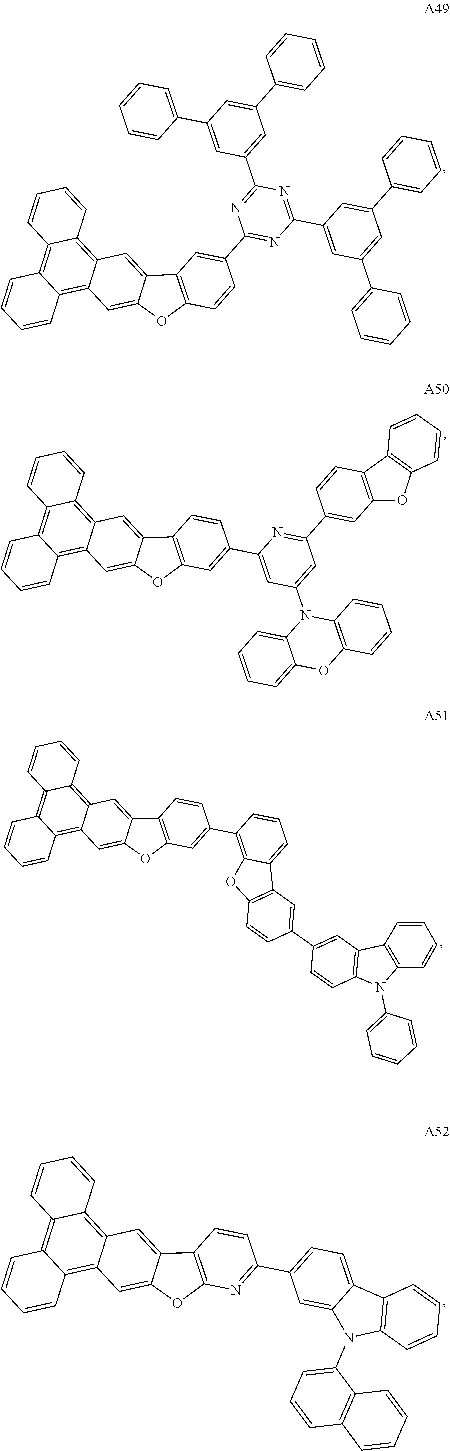 [SMILES: c1ccc(-c2cc(-c3ccccc3)cc(-c3nc(-c4cc(-c5ccccc5)cc(-c5ccccc5)c4)nc(-c4ccc5oc6cc7c8ccccc8c8ccccc8c7cc6c5c4)n3)c2)cc1 ; c1ccc2c(c1)Oc1ccccc1N2c1cc(-c2ccc3c(c2)oc2ccccc23)nc(-c2ccc3c(c2)oc2cc4c5ccccc5c5ccccc5c4cc23)c1 ; c1ccc(-n2c3ccccc3c3cc(-c4ccc5oc6c(-c7ccc8c(c7)oc7cc9c%10ccccc%10c%10ccccc%10c9cc78)cccc6c5c4)ccc32)cc1 ; c1ccc2c(-n3c4ccccc4c4ccc(-c5ccc6c(n5)oc5cc7c8ccccc8c8ccccc8c7cc56)cc43)cccc2c1]

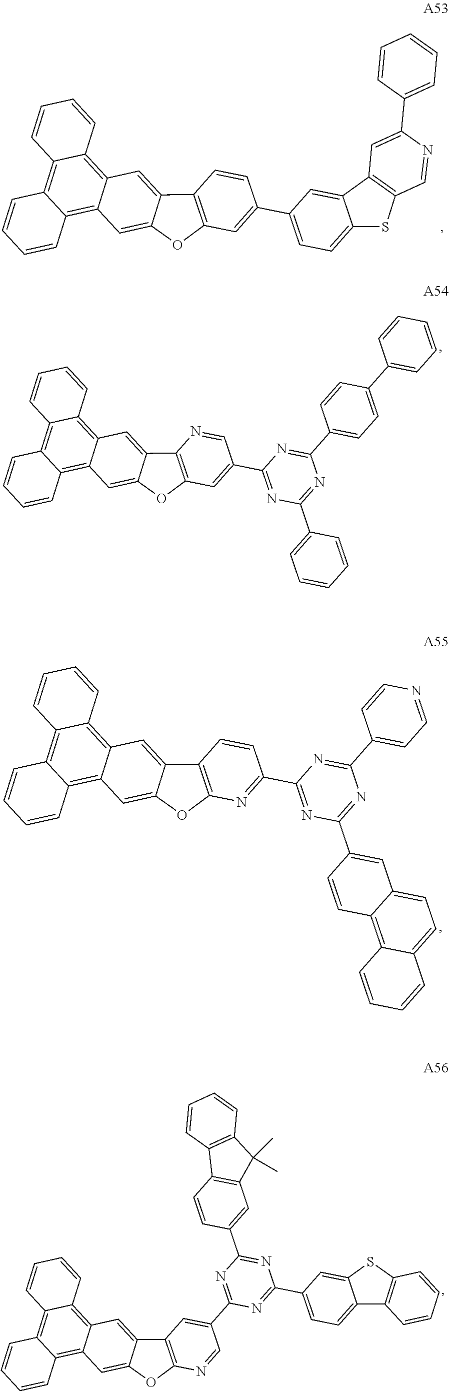 [SMILES: c1ccc(-c2cc3c(cn2)sc2ccc(-c4ccc5c(c4)oc4cc6c7ccccc7c7ccccc7c6cc45)cc23)cc1 ; c1ccc(-c2ccc(-c3nc(-c4ccccc4)nc(-c4cnc5c(c4)oc4cc6c7ccccc7c7ccccc7c6cc45)n3)cc2)cc1 ; c1ccc2c(c1)ccc1cc(-c3nc(-c4ccncc4)nc(-c4ccc5c(n4)oc4cc6c7ccccc7c7ccccc7c6cc45)n3)ccc12 ; CC1(C)c2ccccc2-c2ccc(-c3nc(-c4ccc5c(c4)sc4ccccc45)nc(-c4cnc5oc6cc7c8ccccc8c8ccccc8c7cc6c5c4)n3)cc21]

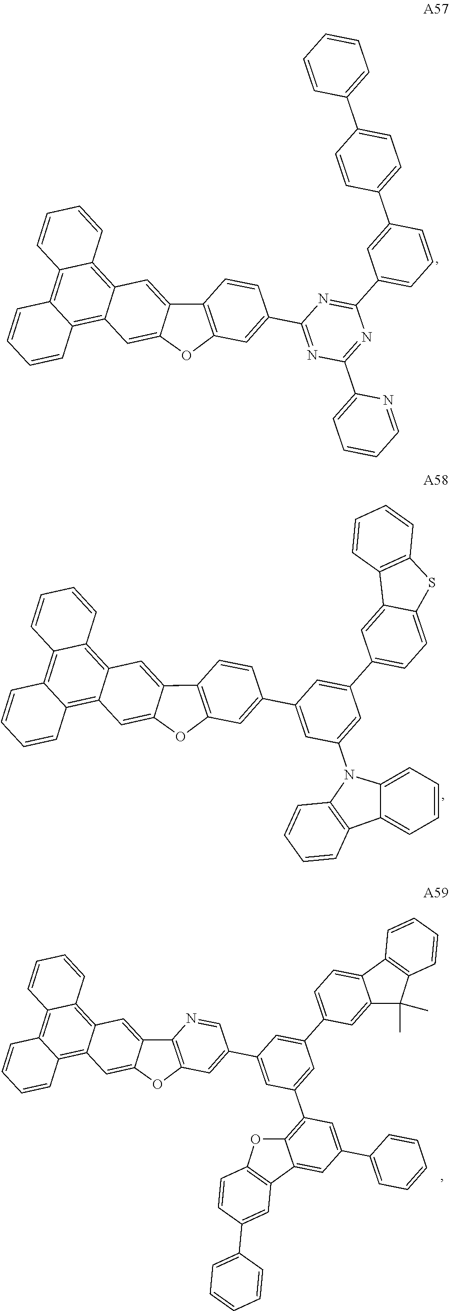 [SMILES: c1ccc(-c2ccc(-c3cccc(-c4nc(-c5ccc6c(c5)oc5cc7c8ccccc8c8ccccc8c7cc56)nc(-c5ccccn5)n4)c3)cc2)cc1 ; c1ccc2c(c1)sc1ccc(-c3cc(-c4ccc5c(c4)oc4cc6c7ccccc7c7ccccc7c6cc45)cc(-n4c5ccccc5c5ccccc54)c3)cc12 ; CC1(C)c2ccccc2-c2ccc(-c3cc(-c4cnc5c(c4)oc4cc6c7ccccc7c7ccccc7c6cc45)cc(-c4cc(-c5ccccc5)cc5c4oc4ccc(-c6ccccc6)cc45)c3)cc21]

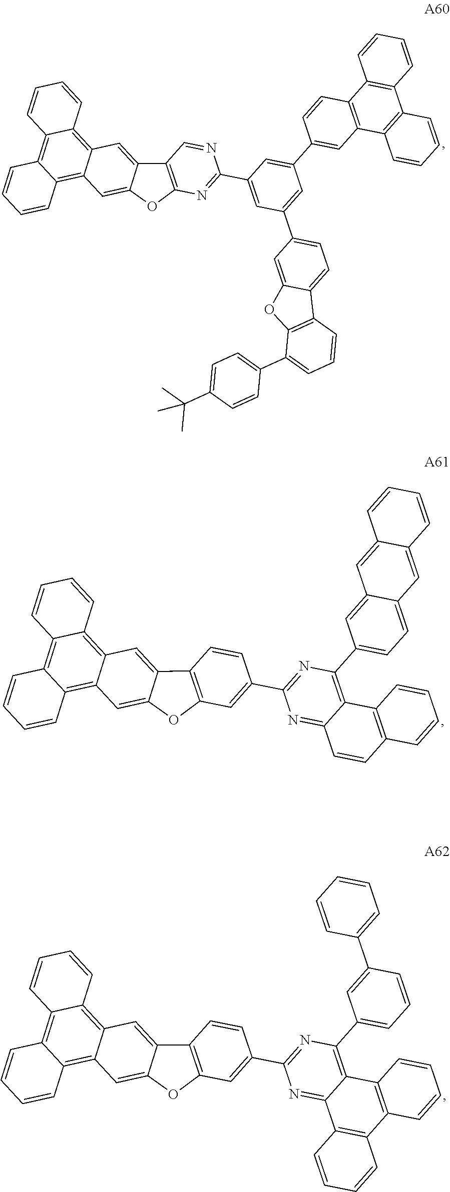 [SMILES: CC(C)(C)c1ccc(-c2cccc3c2oc2cc(-c4cc(-c5ccc6c7ccccc7c7ccccc7c6c5)cc(-c5ncc6c(n5)oc5cc7c8ccccc8c8ccccc8c7cc56)c4)ccc23)cc1 ; c1ccc2cc3cc(-c4nc(-c5ccc6c(c5)oc5cc7c8ccccc8c8ccccc8c7cc56)nc5ccc6ccccc6c45)ccc3cc2c1 ; c1ccc(-c2cccc(-c3nc(-c4ccc5c(c4)oc4cc6c7ccccc7c7ccccc7c6cc45)nc4c5ccccc5c5ccccc5c34)c2)cc1]

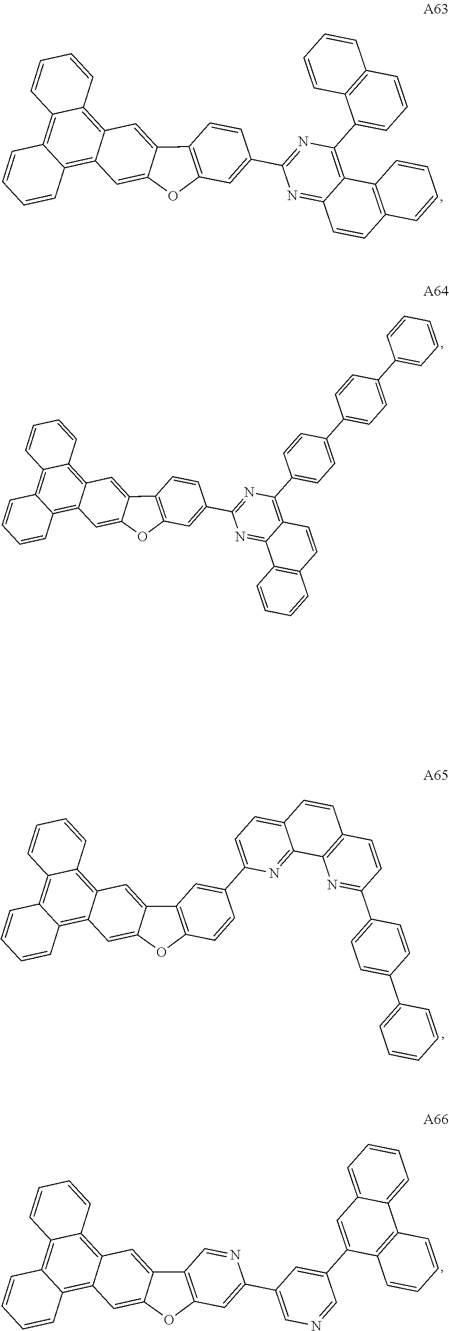 [SMILES: c1ccc2c(-c3nc(-c4ccc5c(c4)oc4cc6c7ccccc7c7ccccc7c6cc45)nc4ccc5ccccc5c34)cccc2c1 ; c1ccc(-c2ccc(-c3ccc(-c4nc(-c5ccc6c(c5)oc5cc7c8ccccc8c8ccccc8c7cc56)nc5c4ccc4ccccc45)cc3)cc2)cc1 ; c1ccc(-c2ccc(-c3ccc4ccc5ccc(-c6ccc7oc8cc9c%10ccccc%10c%10ccccc%10c9cc8c7c6)nc5c4n3)cc2)cc1 ; c1ccc2c(c1)cc(-c1cncc(-c3cc4oc5cc6c7ccccc7c7ccccc7c6cc5c4cn3)c1)c1ccccc12]

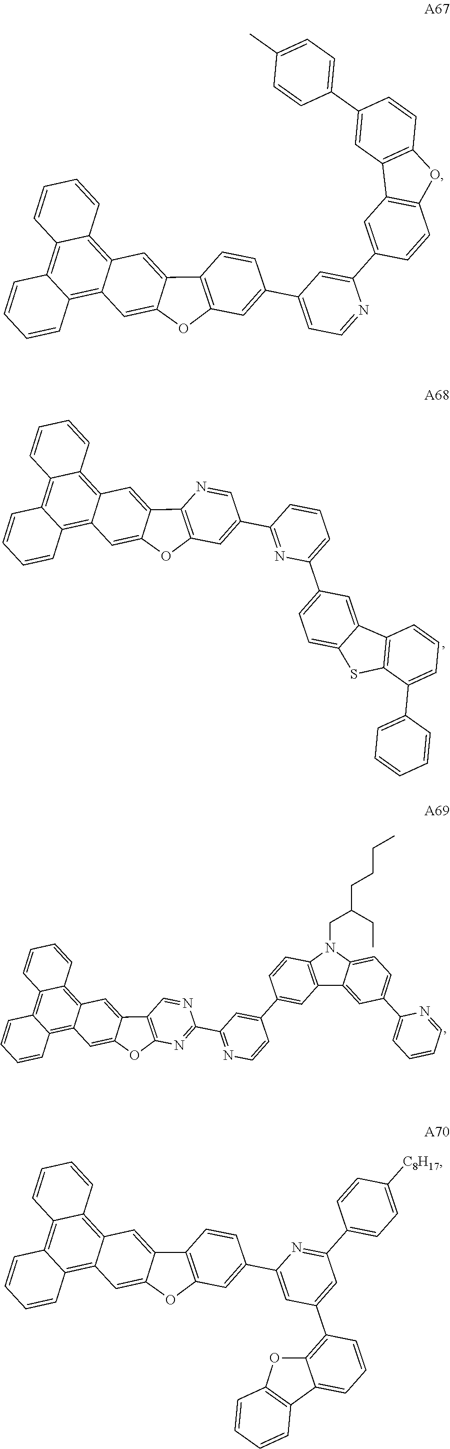

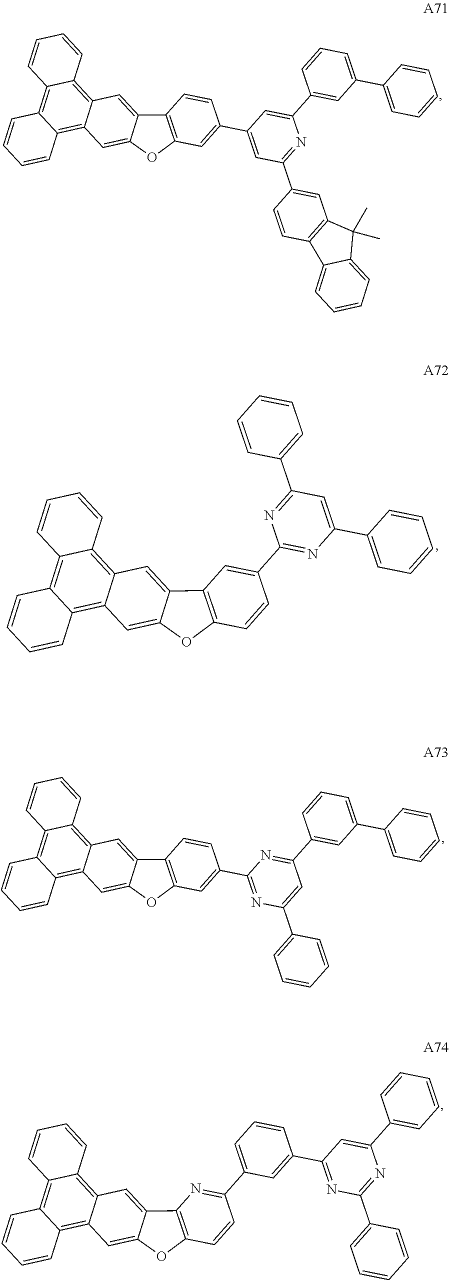 [SMILES: CC1(C)c2ccccc2-c2ccc(-c3cc(-c4ccc5c(c4)oc4cc6c7ccccc7c7ccccc7c6cc45)cc(-c4cccc(-c5ccccc5)c4)n3)cc21 ; c1ccc(-c2cc(-c3ccccc3)nc(-c3ccc4oc5cc6c7ccccc7c7ccccc7c6cc5c4c3)n2)cc1 ; c1ccc(-c2cccc(-c3cc(-c4ccccc4)nc(-c4ccc5c(c4)oc4cc6c7ccccc7c7ccccc7c6cc45)n3)c2)cc1 ; c1ccc(-c2cc(-c3cccc(-c4ccc5oc6cc7c8ccccc8c8ccccc8c7cc6c5n4)c3)nc(-c3ccccc3)n2)cc1]

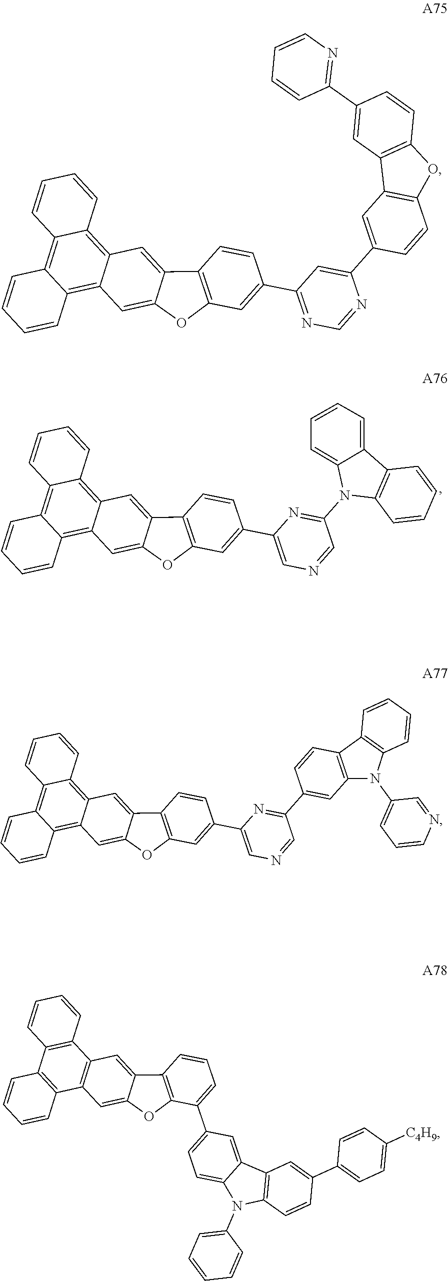

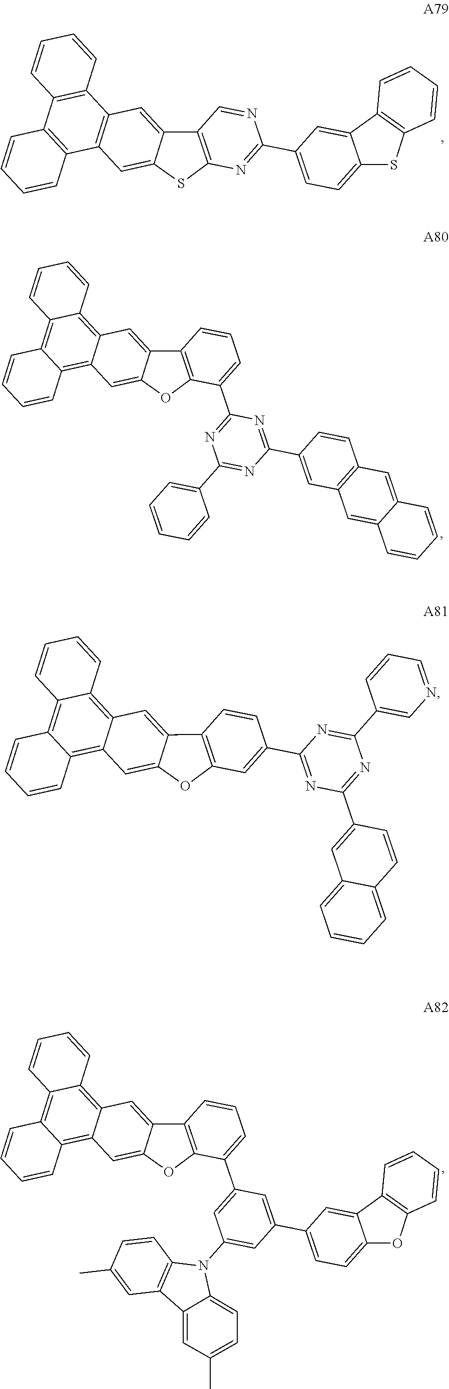 [SMILES: c1ccc2c(c1)sc1ccc(-c3ncc4c(n3)sc3cc5c6ccccc6c6ccccc6c5cc34)cc12 ; c1ccc(-c2nc(-c3ccc4cc5ccccc5cc4c3)nc(-c3cccc4c3oc3cc5c6ccccc6c6ccccc6c5cc34)n2)cc1 ; c1cncc(-c2nc(-c3ccc4ccccc4c3)nc(-c3ccc4c(c3)oc3cc5c6ccccc6c6ccccc6c5cc34)n2)c1 ; Cc1ccc2c(c1)c1cc(C)ccc1n2-c1cc(-c2ccc3oc4ccccc4c3c2)cc(-c2cccc3c2oc2cc4c5ccccc5c5ccccc5c4cc23)c1]

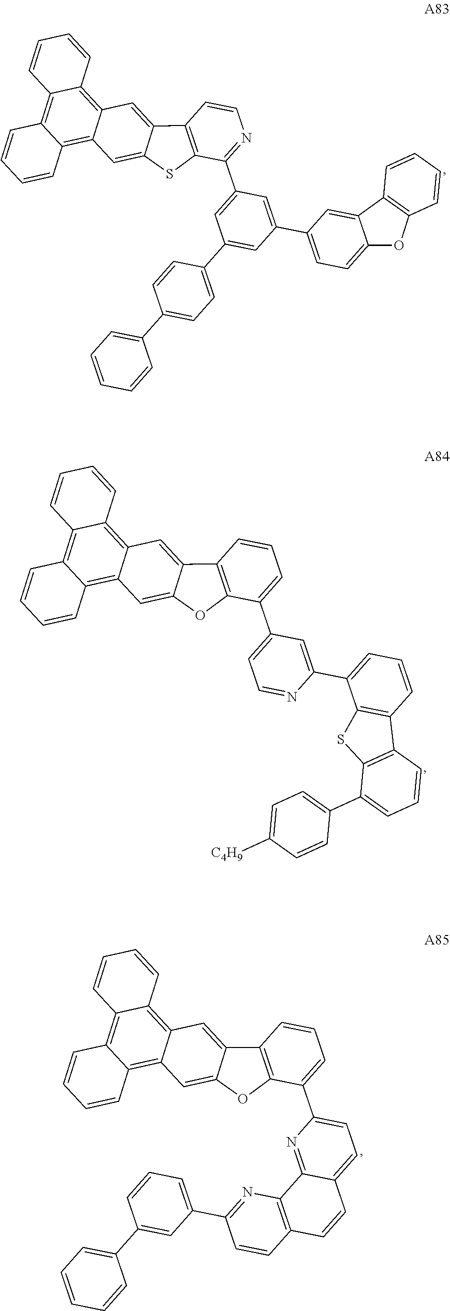

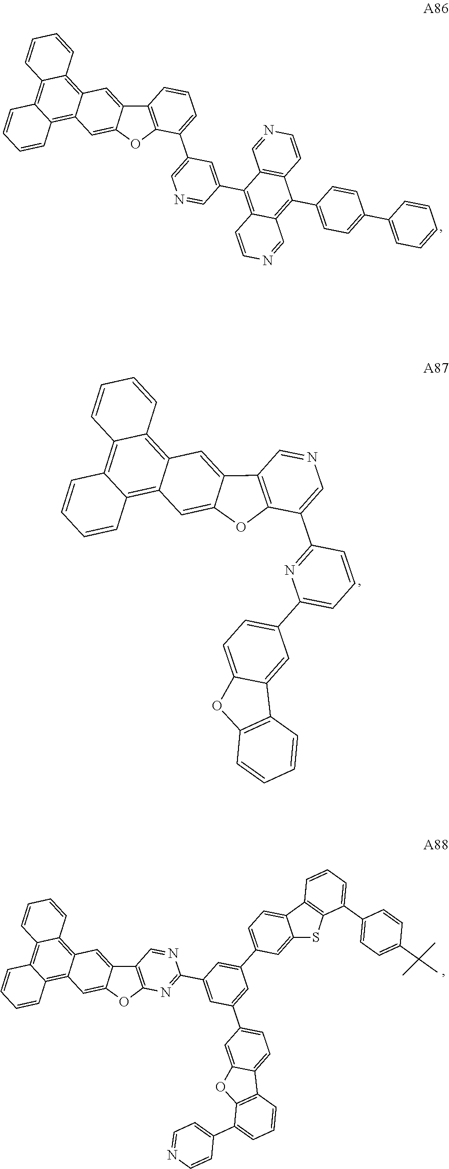 [SMILES: c1ccc(-c2ccc(-c3c4ccncc4c(-c4cncc(-c5cccc6c5oc5cc7c8ccccc8c8ccccc8c7cc56)c4)c4ccncc34)cc2)cc1 ; c1cc(-c2ccc3oc4ccccc4c3c2)nc(-c2cncc3c2oc2cc4c5ccccc5c5ccccc5c4cc23)c1 ; CC(C)(C)c1ccc(-c2cccc3c2sc2cc(-c4cc(-c5ccc6c(c5)oc5c(-c7ccncc7)cccc56)cc(-c5ncc6c(n5)oc5cc7c8ccccc8c8ccccc8c7cc56)c4)ccc23)cc1]

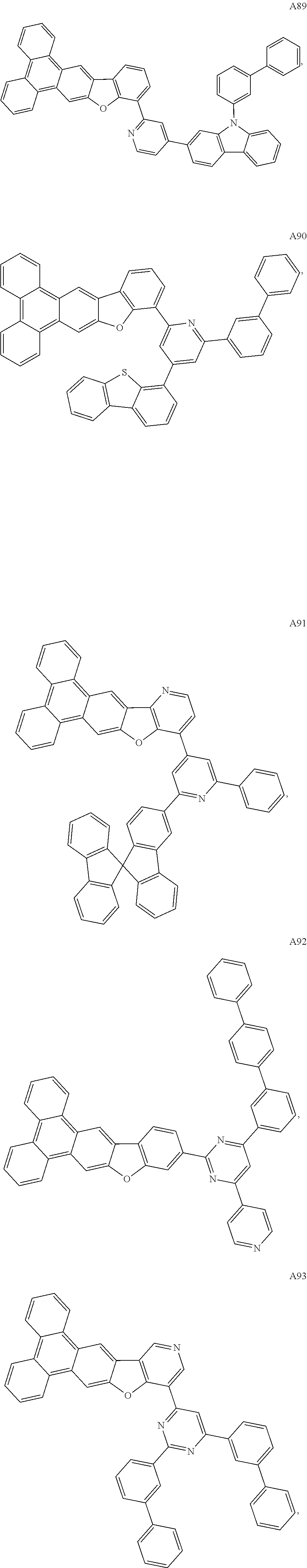 [SMILES: c1ccc(-c2cccc(-n3c4ccccc4c4ccc(-c5ccnc(-c6cccc7c6oc6cc8c9ccccc9c9ccccc9c8cc67)c5)cc43)c2)cc1 ; c1ccc(-c2cccc(-c3cc(-c4cccc5c4sc4ccccc45)cc(-c4cccc5c4oc4cc6c7ccccc7c7ccccc7c6cc45)n3)c2)cc1 ; c1ccc(-c2cc(-c3ccnc4c3oc3cc5c6ccccc6c6ccccc6c5cc34)cc(-c3ccc4c(c3)-c3ccccc3C43c4ccccc4-c4ccccc43)n2)cc1 ; c1ccc(-c2ccc(-c3cccc(-c4cc(-c5ccncc5)nc(-c5ccc6c(c5)oc5cc7c8ccccc8c8ccccc8c7cc56)n4)c3)cc2)cc1 ; c1ccc(-c2cccc(-c3cc(-c4cncc5c4oc4cc6c7ccccc7c7ccccc7c6cc45)nc(-c4cccc(-c5ccccc5)c4)n3)c2)cc1]

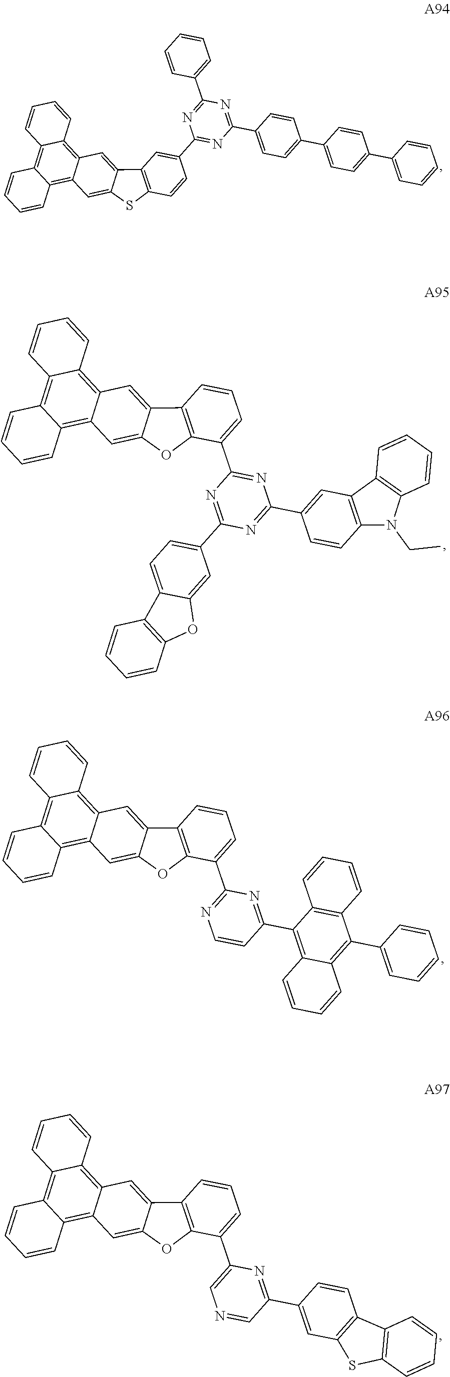 [SMILES: c1ccc(-c2ccc(-c3ccc(-c4nc(-c5ccccc5)nc(-c5ccc6sc7cc8c9ccccc9c9ccccc9c8cc7c6c5)n4)cc3)cc2)cc1 ; CCn1c2ccccc2c2cc(-c3nc(-c4ccc5c(c4)oc4ccccc45)nc(-c4cccc5c4oc4cc6c7ccccc7c7ccccc7c6cc45)n3)ccc21 ; c1ccc(-c2c3ccccc3c(-c3ccnc(-c4cccc5c4oc4cc6c7ccccc7c7ccccc7c6cc45)n3)c3ccccc23)cc1 ; c1ccc2c(c1)sc1cc(-c3cncc(-c4cccc5c4oc4cc6c7ccccc7c7ccccc7c6cc45)n3)ccc12]

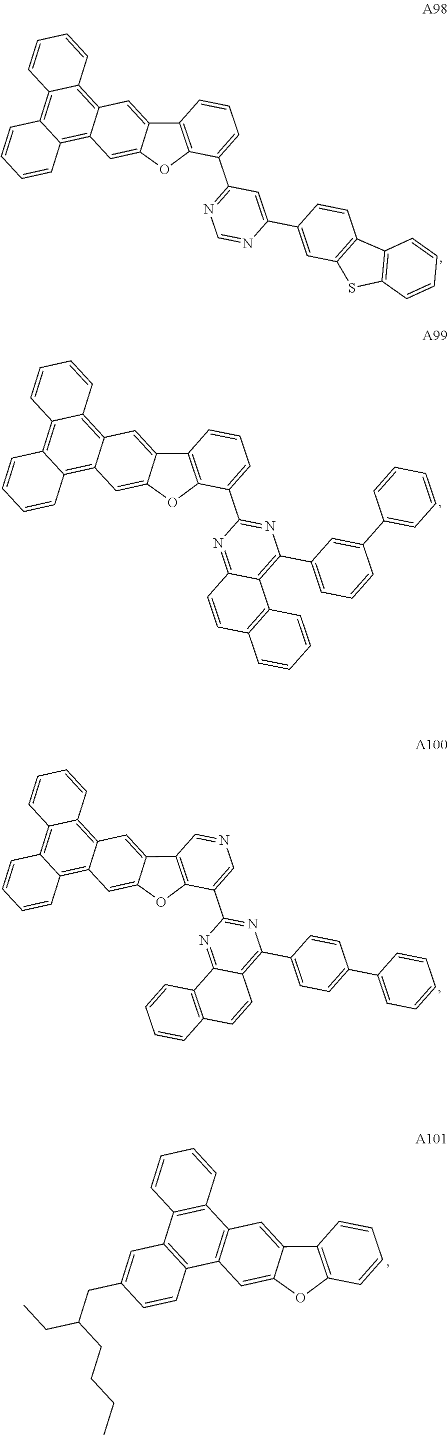 [SMILES: c1ccc2c(c1)sc1cc(-c3cc(-c4cccc5c4oc4cc6c7ccccc7c7ccccc7c6cc45)ncn3)ccc12 ; c1ccc(-c2cccc(-c3nc(-c4cccc5c4oc4cc6c7ccccc7c7ccccc7c6cc45)nc4ccc5ccccc5c34)c2)cc1 ; c1ccc(-c2ccc(-c3nc(-c4cncc5c4oc4cc6c7ccccc7c7ccccc7c6cc45)nc4c3ccc3ccccc34)cc2)cc1 ; CCCCC(CC)Cc1ccc2c(c1)c1ccccc1c1cc3c(cc21)oc1ccccc13]

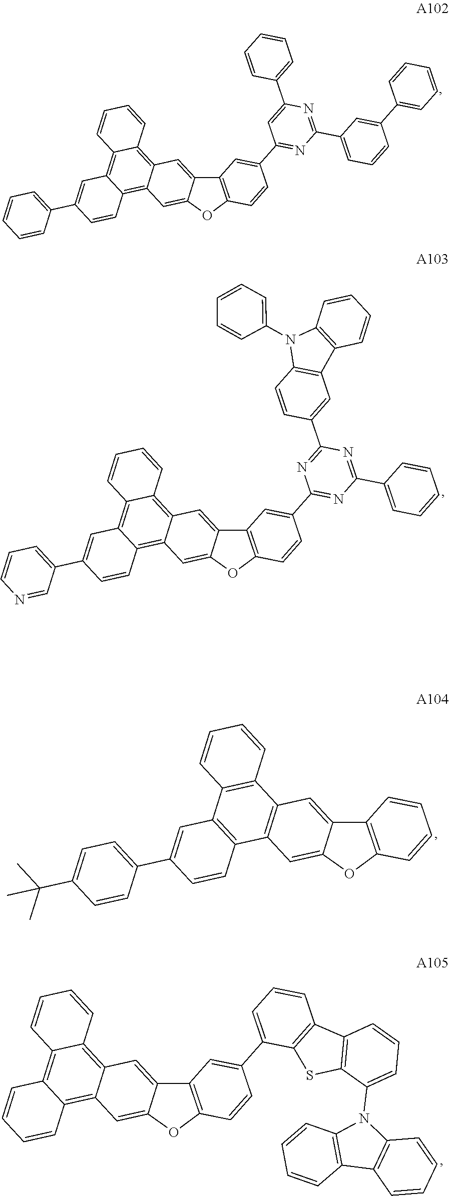 [SMILES: c1ccc(-c2cccc(-c3nc(-c4ccccc4)cc(-c4ccc5oc6cc7c8ccc(-c9ccccc9)cc8c8ccccc8c7cc6c5c4)n3)c2)cc1 ; c1ccc(-c2nc(-c3ccc4oc5cc6c7ccc(-c8cccnc8)cc7c7ccccc7c6cc5c4c3)nc(-c3ccc4c(c3)c3ccccc3n4-c3ccccc3)n2)cc1 ; CC(C)(C)c1ccc(-c2ccc3c(c2)c2ccccc2c2cc4c(cc32)oc2ccccc24)cc1 ; c1cc(-c2ccc3oc4cc5c6ccccc6c6ccccc6c5cc4c3c2)c2sc3c(-n4c5ccccc5c5ccccc54)cccc3c2c1]

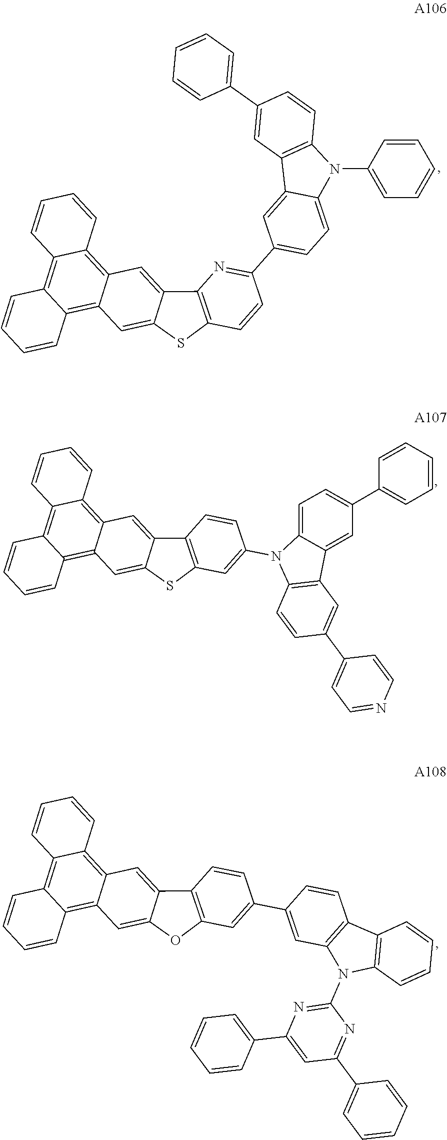 [SMILES: c1ccc(-c2ccc3c(c2)c2cc(-c4ccc5sc6cc7c8ccccc8c8ccccc8c7cc6c5n4)ccc2n3-c2ccccc2)cc1 ; c1ccc(-c2ccc3c(c2)c2cc(-c4ccncc4)ccc2n3-c2ccc3c(c2)sc2cc4c5ccccc5c5ccccc5c4cc23)cc1 ; c1ccc(-c2cc(-c3ccccc3)nc(-n3c4ccccc4c4ccc(-c5ccc6c(c5)oc5cc7c8ccccc8c8ccccc8c7cc56)cc43)n2)cc1]

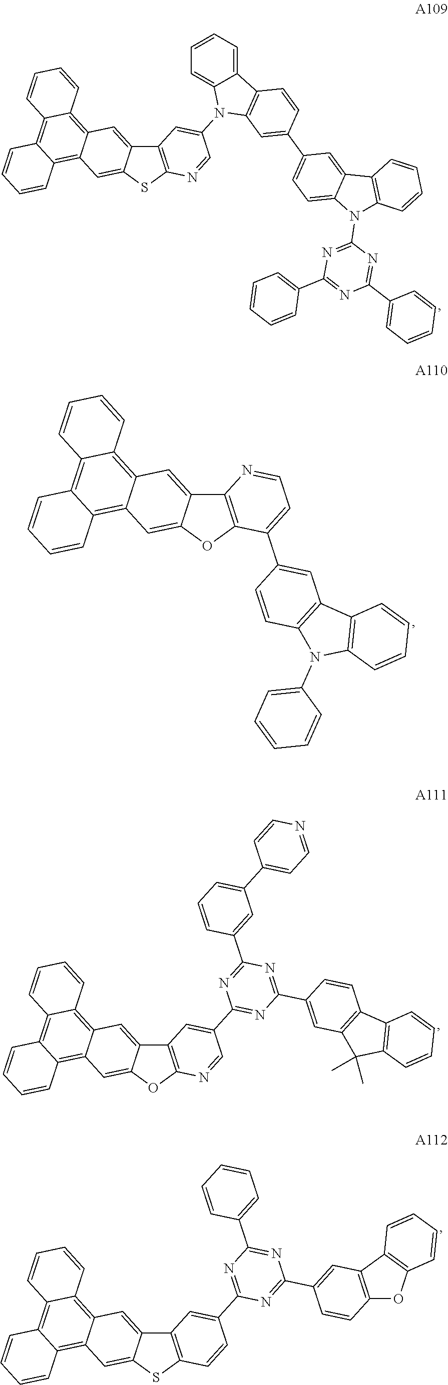 [SMILES: c1ccc(-c2nc(-c3ccccc3)nc(-n3c4ccccc4c4cc(-c5ccc6c7ccccc7n(-c7cnc8sc9cc%10c%11ccccc%11c%11ccccc%11c%10cc9c8c7)c6c5)ccc43)n2)cc1 ; c1ccc(-n2c3ccccc3c3cc(-c4ccnc5c4oc4cc6c7ccccc7c7ccccc7c6cc45)ccc32)cc1 ; CC1(C)c2ccccc2-c2ccc(-c3nc(-c4cccc(-c5ccncc5)c4)nc(-c4cnc5oc6cc7c8ccccc8c8ccccc8c7cc6c5c4)n3)cc21 ; c1ccc(-c2nc(-c3ccc4oc5ccccc5c4c3)nc(-c3ccc4sc5cc6c7ccccc7c7ccccc7c6cc5c4c3)n2)cc1]

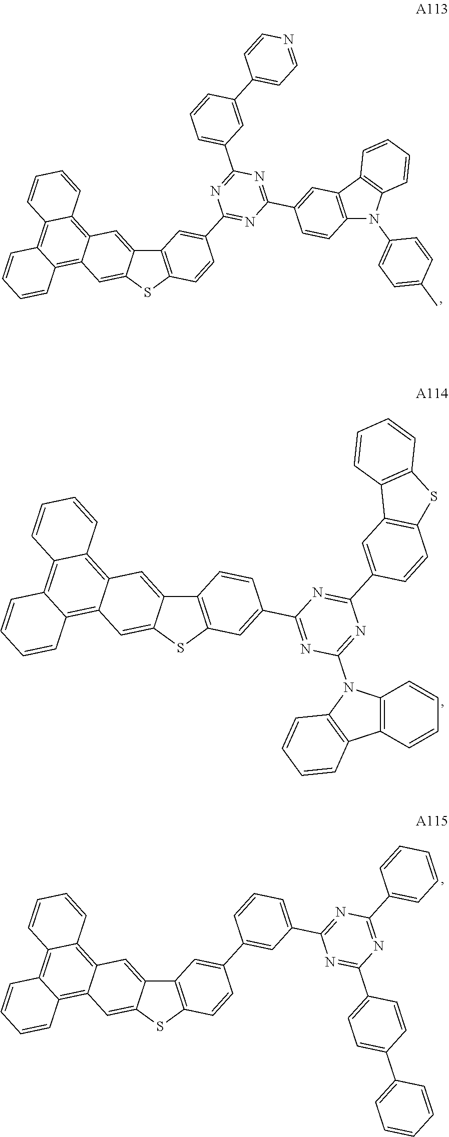 [SMILES: Cc1ccc(-n2c3ccccc3c3cc(-c4nc(-c5cccc(-c6ccncc6)c5)nc(-c5ccc6sc7cc8c9ccccc9c9ccccc9c8cc7c6c5)n4)ccc32)cc1 ; c1ccc2c(c1)sc1ccc(-c3nc(-c4ccc5c(c4)sc4cc6c7ccccc7c7ccccc7c6cc45)nc(-n4c5ccccc5c5ccccc54)n3)cc12 ; c1ccc(-c2ccc(-c3nc(-c4ccccc4)nc(-c4cccc(-c5ccc6sc7cc8c9ccccc9c9ccccc9c8cc7c6c5)c4)n3)cc2)cc1]

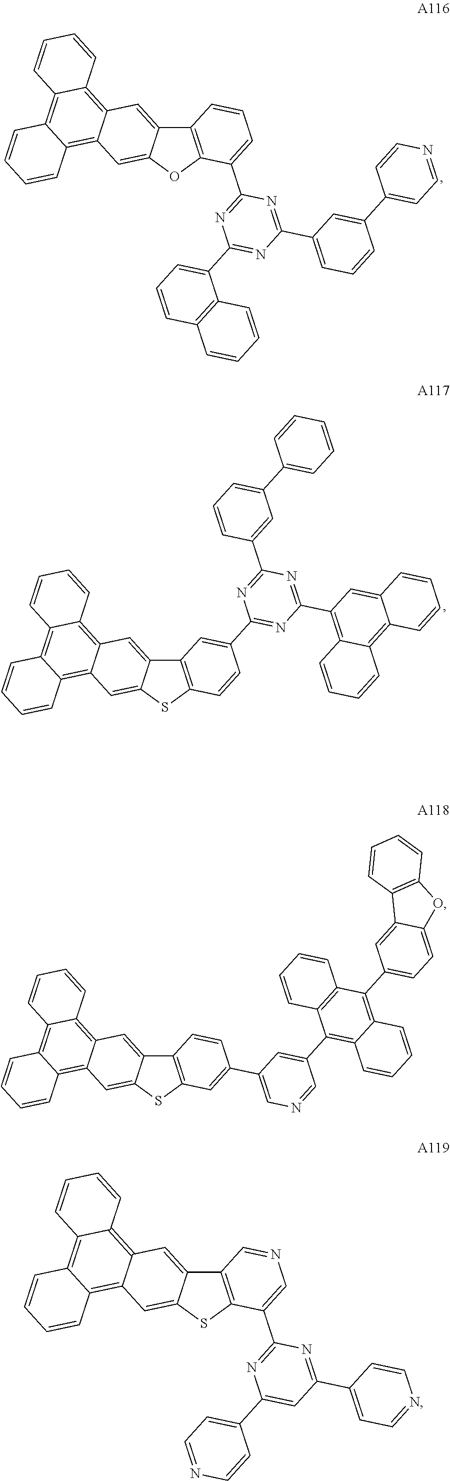 [SMILES: c1cc(-c2ccncc2)cc(-c2nc(-c3cccc4ccccc34)nc(-c3cccc4c3oc3cc5c6ccccc6c6ccccc6c5cc34)n2)c1 ; c1ccc(-c2cccc(-c3nc(-c4ccc5sc6cc7c8ccccc8c8ccccc8c7cc6c5c4)nc(-c4cc5ccccc5c5ccccc45)n3)c2)cc1 ; c1ccc2c(c1)oc1ccc(-c3c4ccccc4c(-c4cncc(-c5ccc6c(c5)sc5cc7c8ccccc8c8ccccc8c7cc56)c4)c4ccccc34)cc12 ; c1ccc2c(c1)c1ccccc1c1cc3c(cc21)sc1c(-c2nc(-c4ccncc4)cc(-c4ccncc4)n2)cncc13]

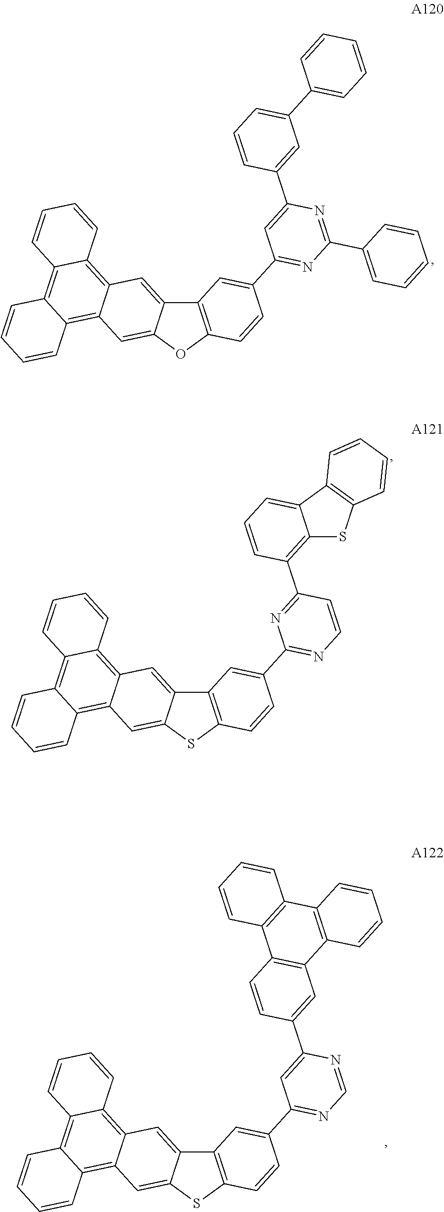 [SMILES: c1ccc(-c2cccc(-c3cc(-c4ccc5oc6cc7c8ccccc8c8ccccc8c7cc6c5c4)nc(-c4ccccc4)n3)c2)cc1 ; c1ccc2c(c1)sc1c(-c3ccnc(-c4ccc5sc6cc7c8ccccc8c8ccccc8c7cc6c5c4)n3)cccc12 ; c1ccc2c(c1)c1ccccc1c1cc(-c3cc(-c4ccc5sc6cc7c8ccccc8c8ccccc8c7cc6c5c4)ncn3)ccc21]

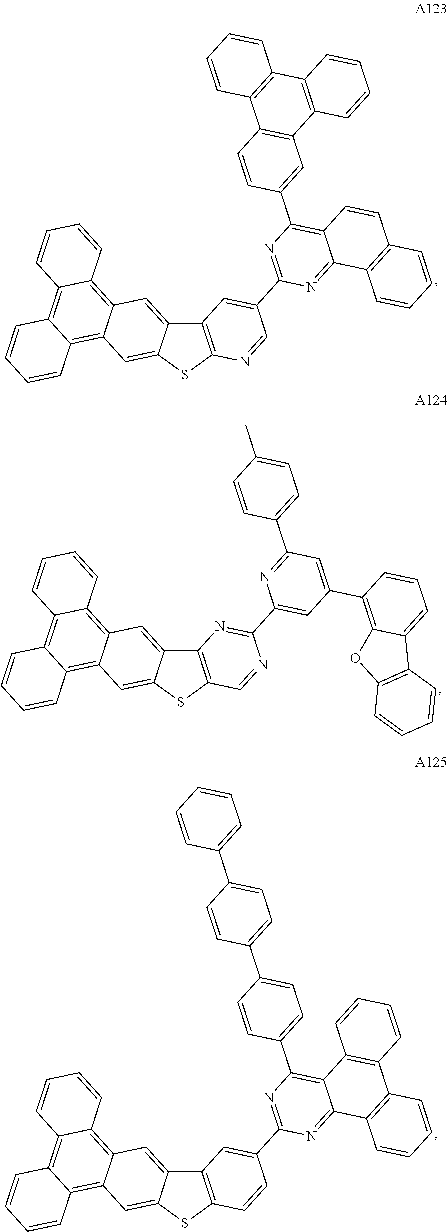 [SMILES: c1ccc2c(c1)ccc1c(-c3ccc4c5ccccc5c5ccccc5c4c3)nc(-c3cnc4sc5cc6c7ccccc7c7ccccc7c6cc5c4c3)nc12 ; Cc1ccc(-c2cc(-c3cccc4c3oc3ccccc34)cc(-c3ncc4sc5cc6c7ccccc7c7ccccc7c6cc5c4n3)n2)cc1 ; c1ccc(-c2ccc(-c3ccc(-c4nc(-c5ccc6sc7cc8c9ccccc9c9ccccc9c8cc7c6c5)nc5c6ccccc6c6ccccc6c45)cc3)cc2)cc1]

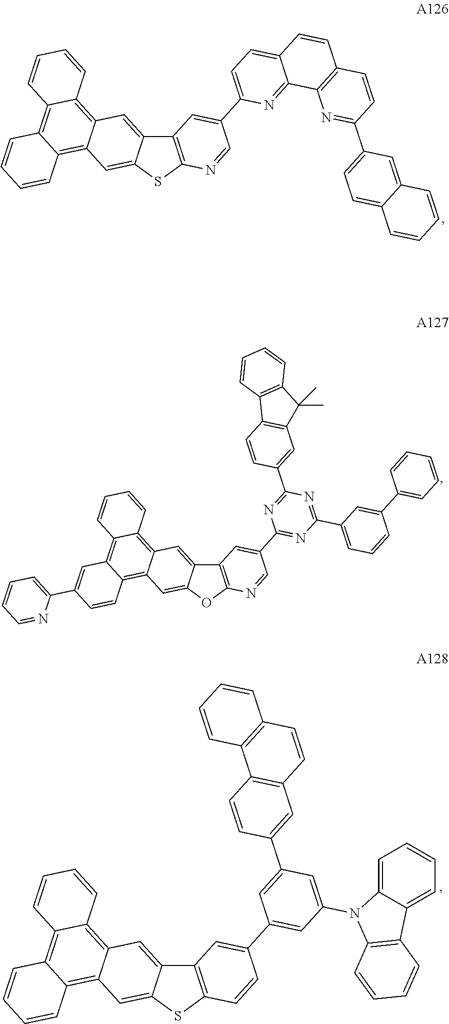 [SMILES: c1ccc2cc(-c3ccc4ccc5ccc(-c6cnc7sc8cc9c%10ccccc%10c%10ccccc%10c9cc8c7c6)nc5c4n3)ccc2c1 ; CC1(C)c2ccccc2-c2ccc(-c3nc(-c4cccc(-c5ccccc5)c4)nc(-c4cnc5oc6cc7c8ccc(-c9ccccn9)cc8c8ccccc8c7cc6c5c4)n3)cc21 ; c1ccc2c(c1)ccc1cc(-c3cc(-c4ccc5sc6cc7c8ccccc8c8ccccc8c7cc6c5c4)cc(-n4c5ccccc5c5ccccc54)c3)ccc12]

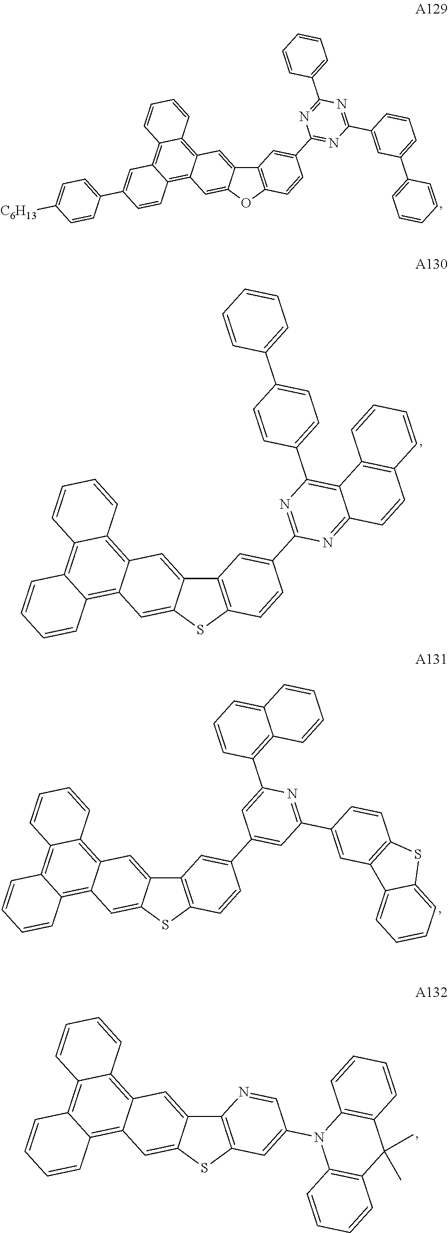

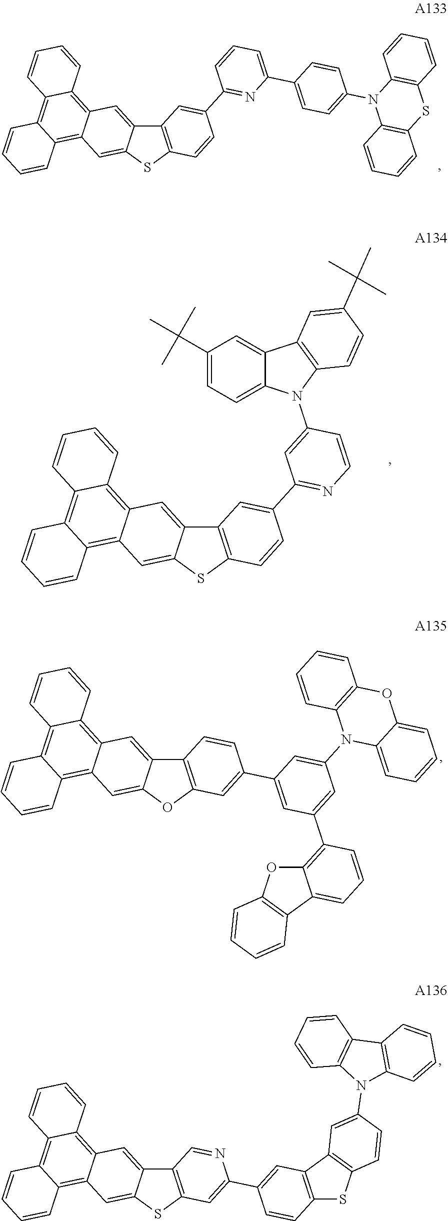 [SMILES: c1cc(-c2ccc(N3c4ccccc4Sc4ccccc43)cc2)nc(-c2ccc3sc4cc5c6ccccc6c6ccccc6c5cc4c3c2)c1 ; CC(C)(C)c1ccc2c(c1)c1cc(C(C)(C)C)ccc1n2-c1ccnc(-c2ccc3sc4cc5c6ccccc6c6ccccc6c5cc4c3c2)c1 ; c1ccc2c(c1)Oc1ccccc1N2c1cc(-c2ccc3c(c2)oc2cc4c5ccccc5c5ccccc5c4cc23)cc(-c2cccc3c2oc2ccccc23)c1 ; c1ccc2c(c1)c1ccccc1c1cc3c(cc21)sc1cc(-c2ccc4sc5ccc(-n6c7ccccc7c7ccccc76)cc5c4c2)ncc13]

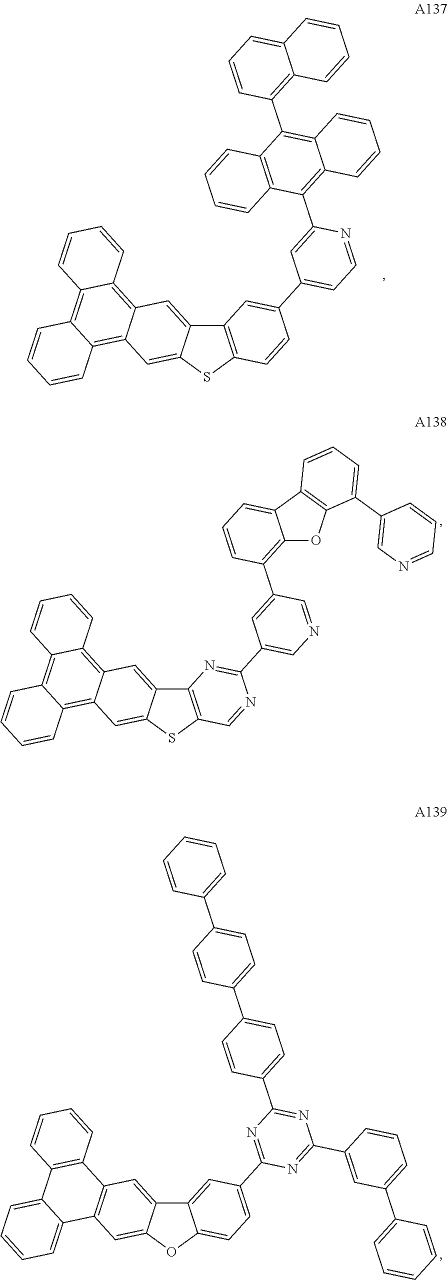 [SMILES: c1ccc2c(-c3c4ccccc4c(-c4cc(-c5ccc6sc7cc8c9ccccc9c9ccccc9c8cc7c6c5)ccn4)c4ccccc34)cccc2c1 ; c1cncc(-c2cccc3c2oc2c(-c4cncc(-c5ncc6sc7cc8c9ccccc9c9ccccc9c8cc7c6n5)c4)cccc23)c1 ; c1ccc(-c2ccc(-c3ccc(-c4nc(-c5cccc(-c6ccccc6)c5)nc(-c5ccc6oc7cc8c9ccccc9c9ccccc9c8cc7c6c5)n4)cc3)cc2)cc1]

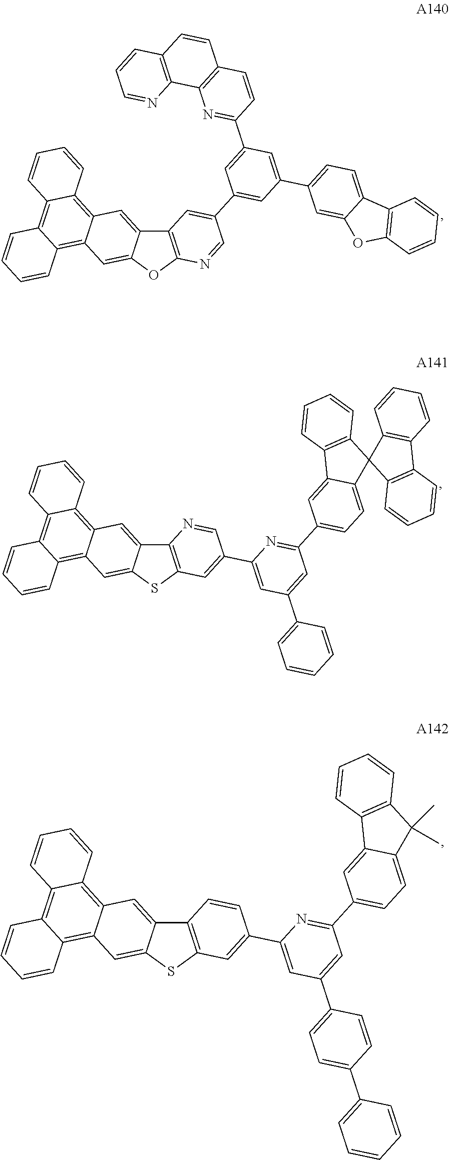 [SMILES: c1cnc2c(c1)ccc1ccc(-c3cc(-c4ccc5c(c4)oc4ccccc45)cc(-c4cnc5oc6cc7c8ccccc8c8ccccc8c7cc6c5c4)c3)nc12 ; c1ccc(-c2cc(-c3ccc4c(c3)-c3ccccc3C43c4ccccc4-c4ccccc43)nc(-c3cnc4c(c3)sc3cc5c6ccccc6c6ccccc6c5cc34)c2)cc1 ; CC1(C)c2ccccc2-c2cc(-c3cc(-c4ccc(-c5ccccc5)cc4)cc(-c4ccc5c(c4)sc4cc6c7ccccc7c7ccccc7c6cc45)n3)ccc21]

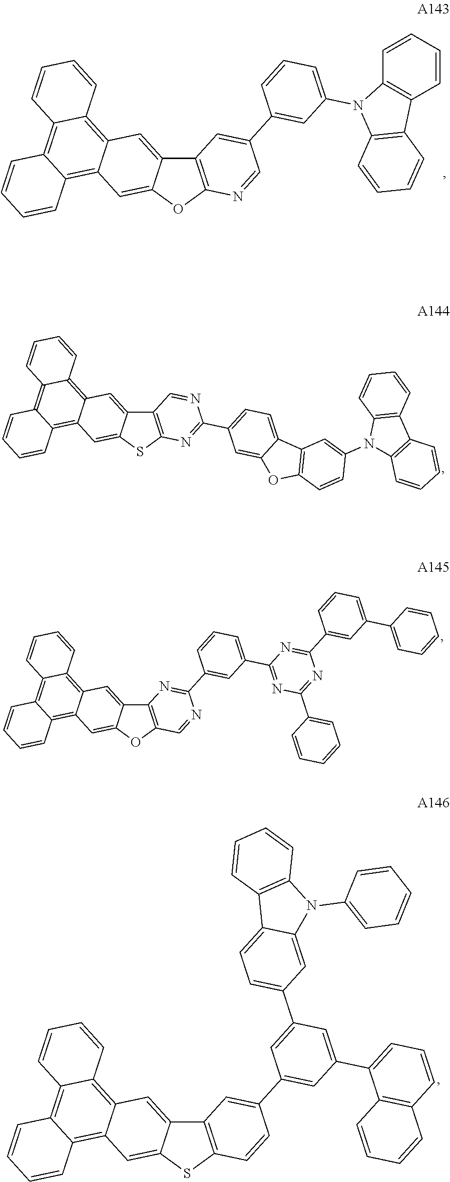 [SMILES: c1cc(-c2cnc3oc4cc5c6ccccc6c6ccccc6c5cc4c3c2)cc(-n2c3ccccc3c3ccccc32)c1 ; c1ccc2c(c1)c1ccccc1c1cc3c(cc21)sc1nc(-c2ccc4c(c2)oc2ccc(-n5c6ccccc6c6ccccc65)cc24)ncc13 ; c1ccc(-c2cccc(-c3nc(-c4ccccc4)nc(-c4cccc(-c5ncc6oc7cc8c9ccccc9c9ccccc9c8cc7c6n5)c4)n3)c2)cc1 ; c1ccc(-n2c3ccccc3c3ccc(-c4cc(-c5ccc6sc7cc8c9ccccc9c9ccccc9c8cc7c6c5)cc(-c5cccc6ccccc56)c4)cc32)cc1]

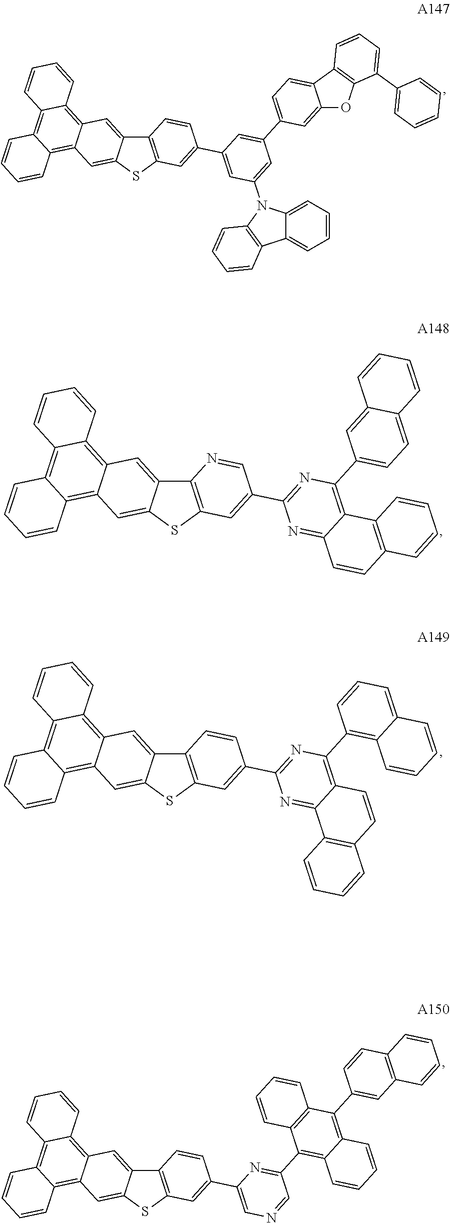 [SMILES: c1ccc(-c2cccc3c2oc2cc(-c4cc(-c5ccc6c(c5)sc5cc7c8ccccc8c8ccccc8c7cc56)cc(-n5c6ccccc6c6ccccc65)c4)ccc23)cc1 ; c1ccc2cc(-c3nc(-c4cnc5c(c4)sc4cc6c7ccccc7c7ccccc7c6cc45)nc4ccc5ccccc5c34)ccc2c1 ; c1ccc2c(-c3nc(-c4ccc5c(c4)sc4cc6c7ccccc7c7ccccc7c6cc45)nc4c3ccc3ccccc34)cccc2c1 ; c1ccc2cc(-c3c4ccccc4c(-c4cncc(-c5ccc6c(c5)sc5cc7c8ccccc8c8ccccc8c7cc56)n4)c4ccccc34)ccc2c1]

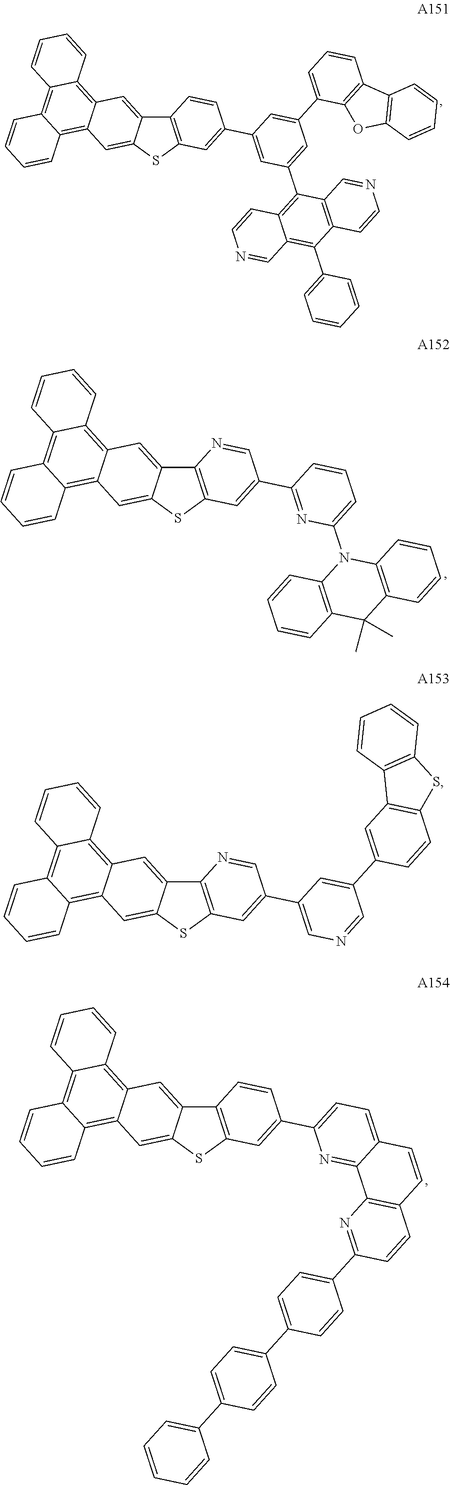 [SMILES: c1ccc(-c2c3ccncc3c(-c3cc(-c4ccc5c(c4)sc4cc6c7ccccc7c7ccccc7c6cc45)cc(-c4cccc5c4oc4ccccc45)c3)c3ccncc23)cc1 ; CC1(C)c2ccccc2N(c2cccc(-c3cnc4c(c3)sc3cc5c6ccccc6c6ccccc6c5cc34)n2)c2ccccc21 ; c1ccc2c(c1)sc1ccc(-c3cncc(-c4cnc5c(c4)sc4cc6c7ccccc7c7ccccc7c6cc45)c3)cc12 ; c1ccc(-c2ccc(-c3ccc(-c4ccc5ccc6ccc(-c7ccc8c(c7)sc7cc9c%10ccccc%10c%10ccccc%10c9cc78)nc6c5n4)cc3)cc2)cc1]

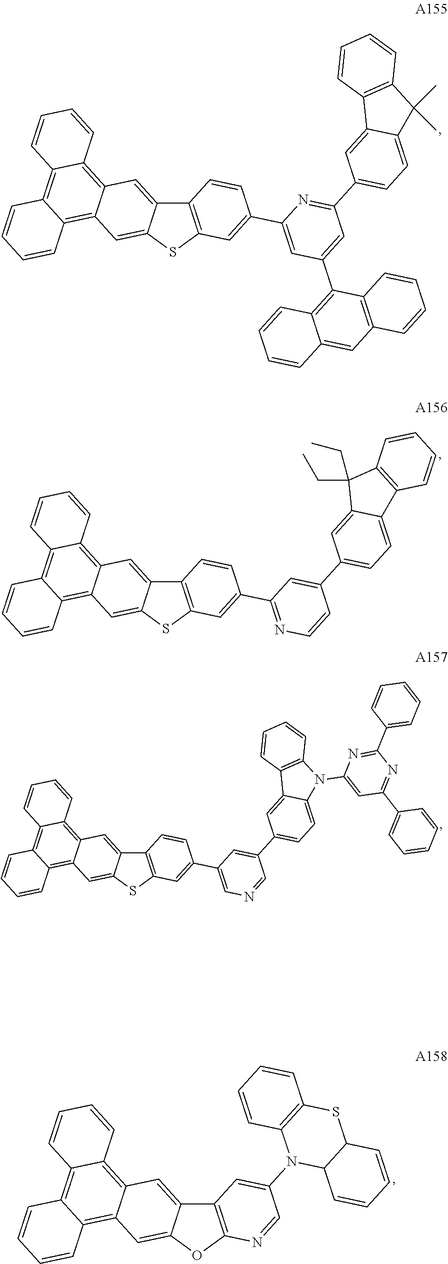 [SMILES: CC1(C)c2ccccc2-c2cc(-c3cc(-c4c5ccccc5cc5ccccc45)cc(-c4ccc5c(c4)sc4cc6c7ccccc7c7ccccc7c6cc45)n3)ccc21 ; CCC1(CC)c2ccccc2-c2ccc(-c3ccnc(-c4ccc5c(c4)sc4cc6c7ccccc7c7ccccc7c6cc45)c3)cc21 ; c1ccc(-c2cc(-n3c4ccccc4c4cc(-c5cncc(-c6ccc7c(c6)sc6cc8c9ccccc9c9ccccc9c8cc67)c5)ccc43)nc(-c3ccccc3)n2)cc1 ; C1=CC2Sc3ccccc3N(c3cnc4oc5cc6c7ccccc7c7ccccc7c6cc5c4c3)C2C=C1]

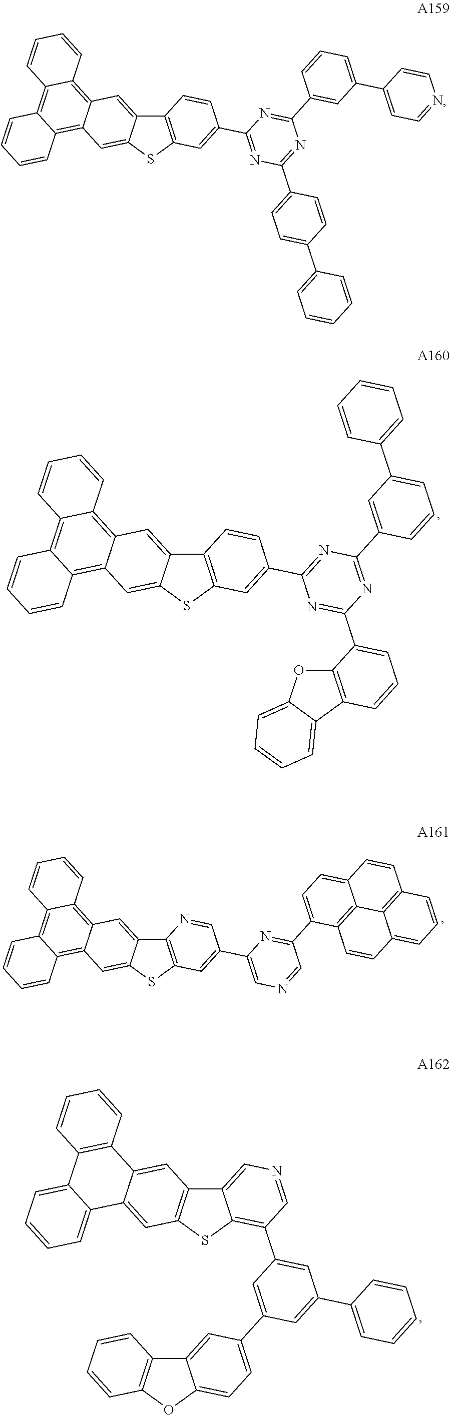 [SMILES: c1ccc(-c2ccc(-c3nc(-c4cccc(-c5ccncc5)c4)nc(-c4ccc5c(c4)sc4cc6c7ccccc7c7ccccc7c6cc45)n3)cc2)cc1 ; c1ccc(-c2cccc(-c3nc(-c4ccc5c(c4)sc4cc6c7ccccc7c7ccccc7c6cc45)nc(-c4cccc5c4oc4ccccc45)n3)c2)cc1 ; c1cc2ccc3ccc(-c4cncc(-c5cnc6c(c5)sc5cc7c8ccccc8c8ccccc8c7cc56)n4)c4ccc(c1)c2c34 ; c1ccc(-c2cc(-c3ccc4oc5ccccc5c4c3)cc(-c3cncc4c3sc3cc5c6ccccc6c6ccccc6c5cc34)c2)cc1]

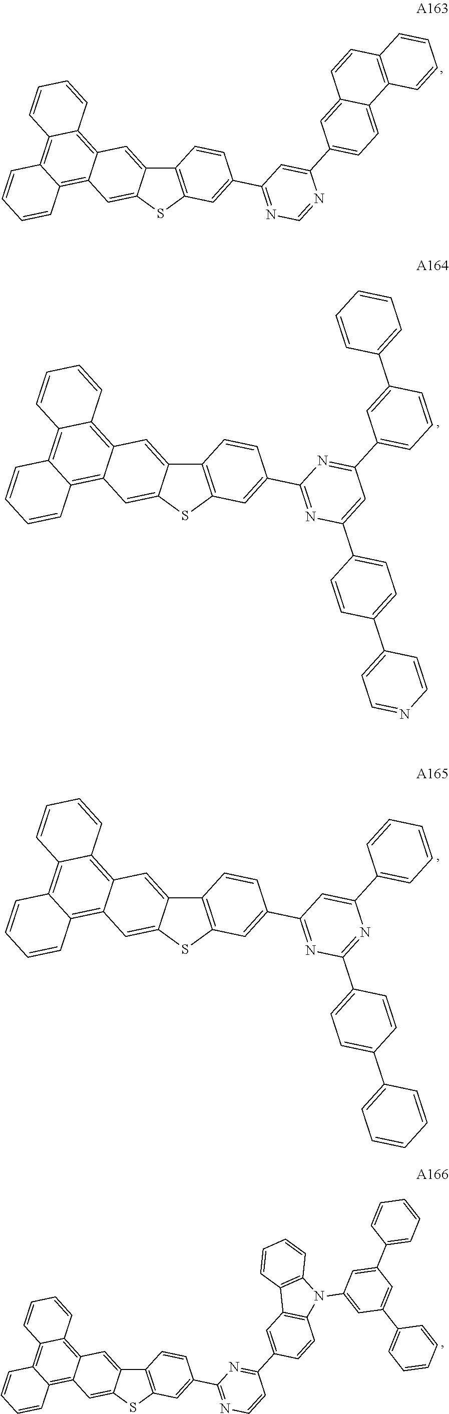 [SMILES: c1ccc2c(c1)ccc1cc(-c3cc(-c4ccc5c(c4)sc4cc6c7ccccc7c7ccccc7c6cc45)ncn3)ccc12 ; c1ccc(-c2cccc(-c3cc(-c4ccc(-c5ccncc5)cc4)nc(-c4ccc5c(c4)sc4cc6c7ccccc7c7ccccc7c6cc45)n3)c2)cc1 ; c1ccc(-c2ccc(-c3nc(-c4ccccc4)cc(-c4ccc5c(c4)sc4cc6c7ccccc7c7ccccc7c6cc45)n3)cc2)cc1 ; c1ccc(-c2cc(-c3ccccc3)cc(-n3c4ccccc4c4cc(-c5ccnc(-c6ccc7c(c6)sc6cc8c9ccccc9c9ccccc9c8cc67)n5)ccc43)c2)cc1]

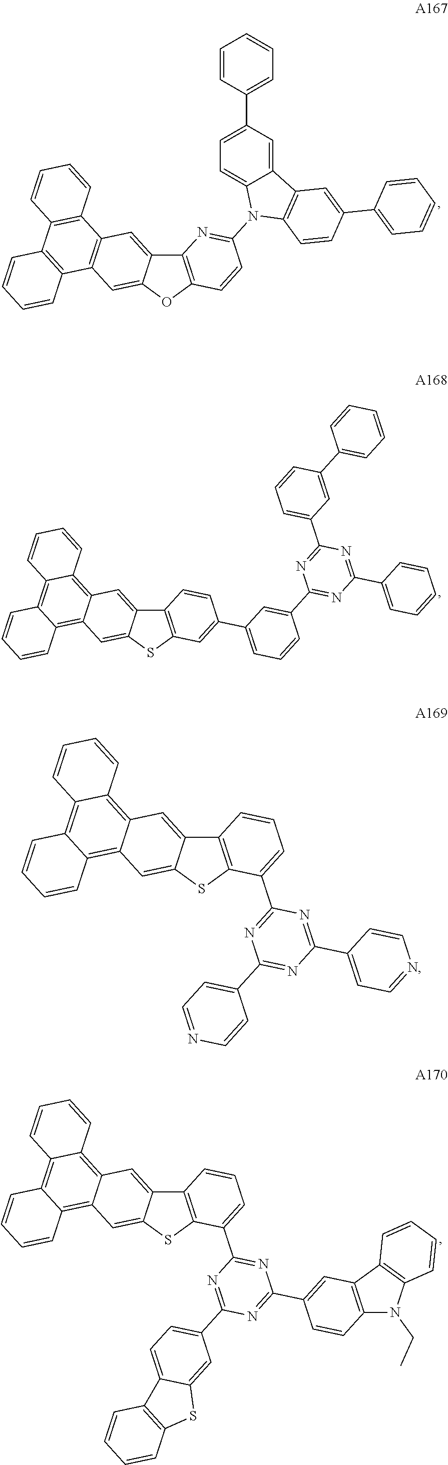 [SMILES: c1ccc(-c2ccc3c(c2)c2cc(-c4ccccc4)ccc2n3-c2ccc3oc4cc5c6ccccc6c6ccccc6c5cc4c3n2)cc1 ; c1ccc(-c2cccc(-c3nc(-c4ccccc4)nc(-c4cccc(-c5ccc6c(c5)sc5cc7c8ccccc8c8ccccc8c7cc56)c4)n3)c2)cc1 ; c1cc(-c2nc(-c3ccncc3)nc(-c3ccncc3)n2)c2sc3cc4c5ccccc5c5ccccc5c4cc3c2c1 ; CCn1c2ccccc2c2cc(-c3nc(-c4ccc5c(c4)sc4ccccc45)nc(-c4cccc5c4sc4cc6c7ccccc7c7ccccc7c6cc45)n3)ccc21]

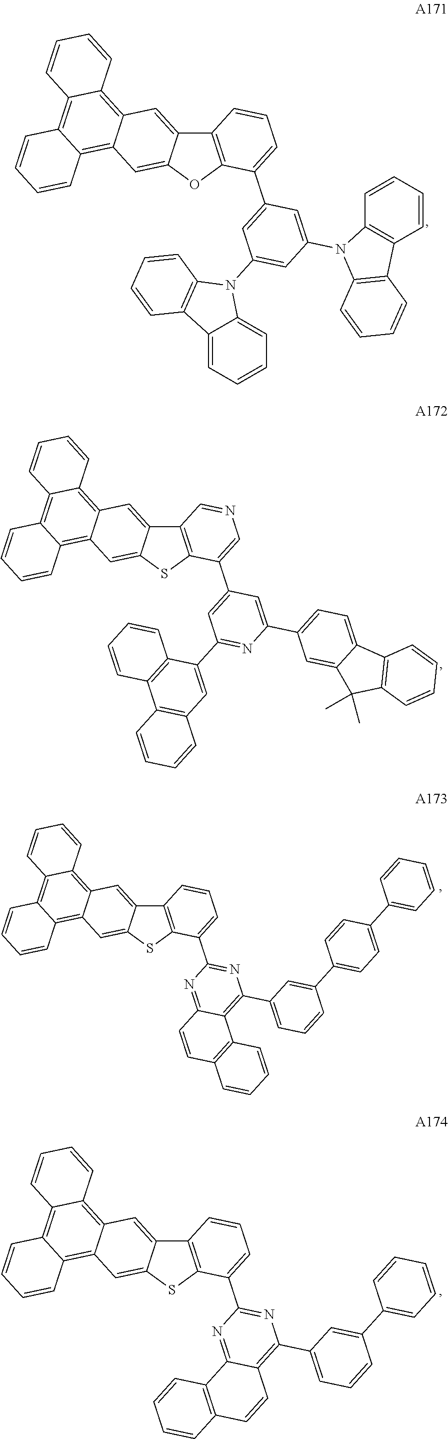 [SMILES: c1cc(-c2cc(-n3c4ccccc4c4ccccc43)cc(-n3c4ccccc4c4ccccc43)c2)c2oc3cc4c5ccccc5c5ccccc5c4cc3c2c1 ; CC1(C)c2ccccc2-c2ccc(-c3cc(-c4cncc5c4sc4cc6c7ccccc7c7ccccc7c6cc45)cc(-c4cc5ccccc5c5ccccc45)n3)cc21 ; c1ccc(-c2ccc(-c3cccc(-c4nc(-c5cccc6c5sc5cc7c8ccccc8c8ccccc8c7cc56)nc5ccc6ccccc6c45)c3)cc2)cc1 ; c1ccc(-c2cccc(-c3nc(-c4cccc5c4sc4cc6c7ccccc7c7ccccc7c6cc45)nc4c3ccc3ccccc34)c2)cc1]

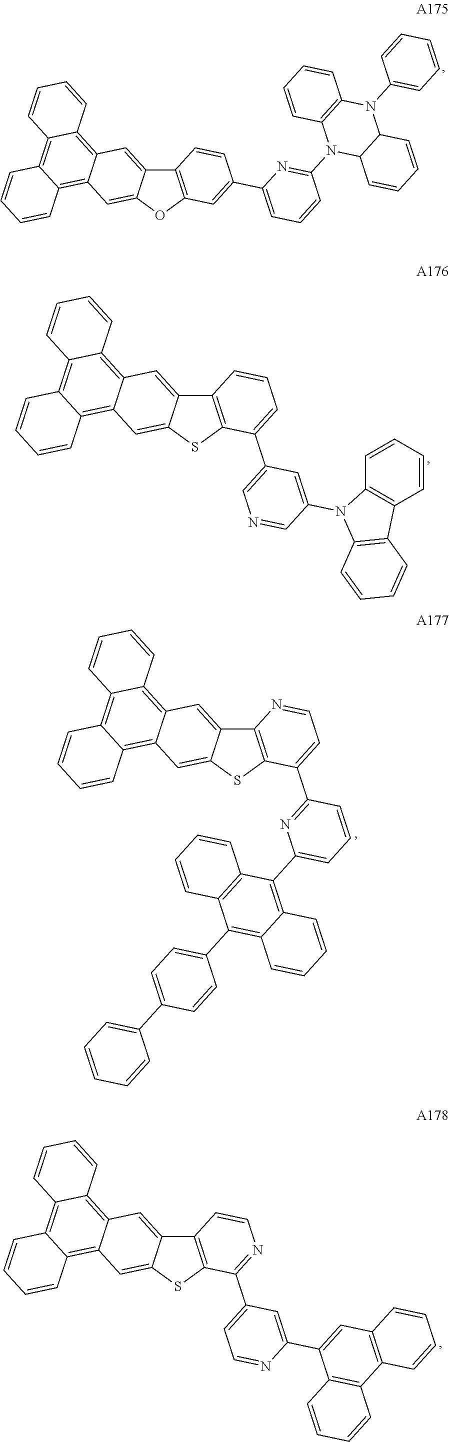 [SMILES: C1=CC2C(C=C1)N(c1cccc(-c3ccc4c(c3)oc3cc5c6ccccc6c6ccccc6c5cc34)n1)c1ccccc1N2c1ccccc1 ; c1cc(-c2cncc(-n3c4ccccc4c4ccccc43)c2)c2sc3cc4c5ccccc5c5ccccc5c4cc3c2c1 ; c1ccc(-c2ccc(-c3c4ccccc4c(-c4cccc(-c5ccnc6c5sc5cc7c8ccccc8c8ccccc8c7cc56)n4)c4ccccc34)cc2)cc1 ; c1ccc2c(c1)cc(-c1cc(-c3nccc4c3sc3cc5c6ccccc6c6ccccc6c5cc34)ccn1)c1ccccc12]

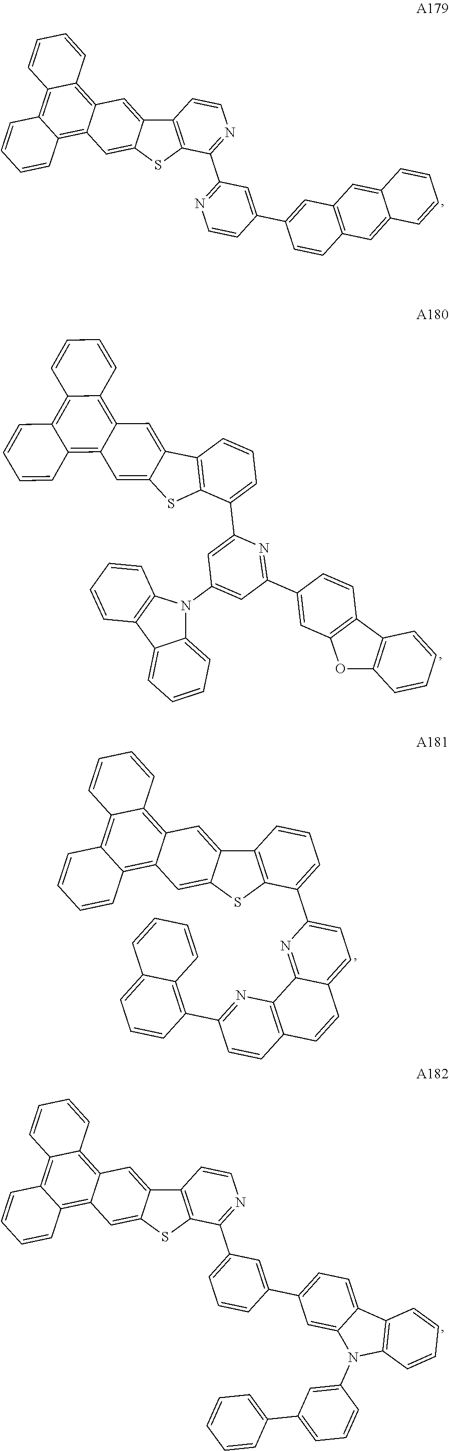 [SMILES: c1ccc2cc3cc(-c4ccnc(-c5nccc6c5sc5cc7c8ccccc8c8ccccc8c7cc56)c4)ccc3cc2c1 ; c1ccc2c(c1)oc1cc(-c3cc(-n4c5ccccc5c5ccccc54)cc(-c4cccc5c4sc4cc6c7ccccc7c7ccccc7c6cc45)n3)ccc12 ; c1ccc2c(-c3ccc4ccc5ccc(-c6cccc7c6sc6cc8c9ccccc9c9ccccc9c8cc67)nc5c4n3)cccc2c1 ; c1ccc(-c2cccc(-n3c4ccccc4c4ccc(-c5cccc(-c6nccc7c6sc6cc8c9ccccc9c9ccccc9c8cc67)c5)cc43)c2)cc1]

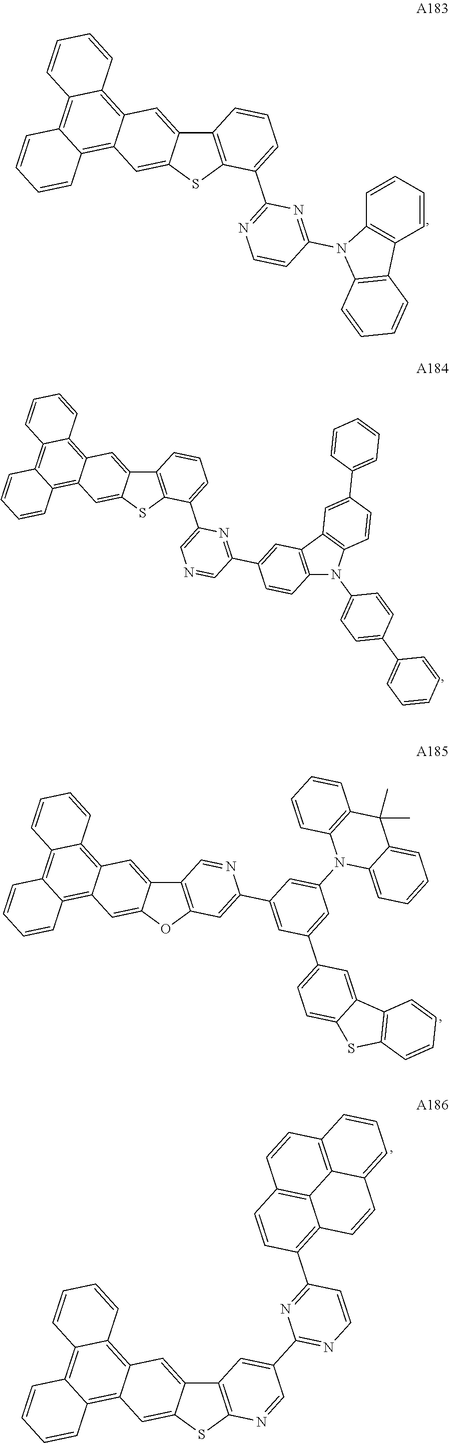 [SMILES: c1cc(-c2nccc(-n3c4ccccc4c4ccccc43)n2)c2sc3cc4c5ccccc5c5ccccc5c4cc3c2c1 ; c1ccc(-c2ccc(-n3c4ccc(-c5ccccc5)cc4c4cc(-c5cncc(-c6cccc7c6sc6cc8c9ccccc9c9ccccc9c8cc67)n5)ccc43)cc2)cc1 ; CC1(C)c2ccccc2N(c2cc(-c3ccc4sc5ccccc5c4c3)cc(-c3cc4oc5cc6c7ccccc7c7ccccc7c6cc5c4cn3)c2)c2ccccc21 ; c1cc2ccc3ccc(-c4ccnc(-c5cnc6sc7cc8c9ccccc9c9ccccc9c8cc7c6c5)n4)c4ccc(c1)c2c34]

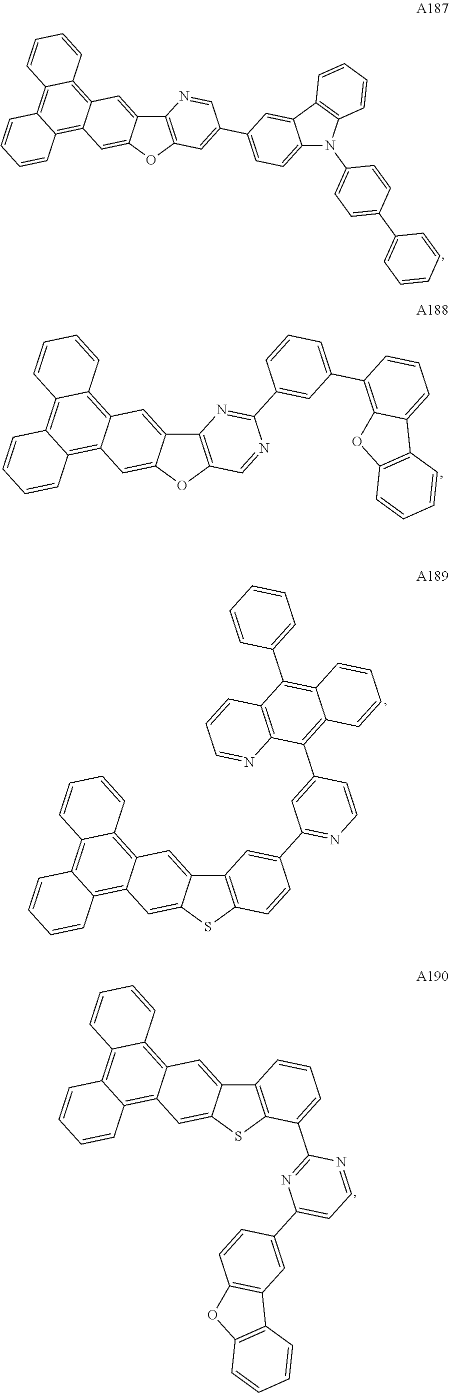 [SMILES: c1ccc(-c2ccc(-n3c4ccccc4c4cc(-c5cnc6c(c5)oc5cc7c8ccccc8c8ccccc8c7cc56)ccc43)cc2)cc1 ; c1cc(-c2ncc3oc4cc5c6ccccc6c6ccccc6c5cc4c3n2)cc(-c2cccc3c2oc2ccccc23)c1 ; c1ccc(-c2c3ccccc3c(-c3ccnc(-c4ccc5sc6cc7c8ccccc8c8ccccc8c7cc6c5c4)c3)c3ncccc23)cc1 ; c1ccc2c(c1)oc1ccc(-c3ccnc(-c4cccc5c4sc4cc6c7ccccc7c7ccccc7c6cc45)n3)cc12]

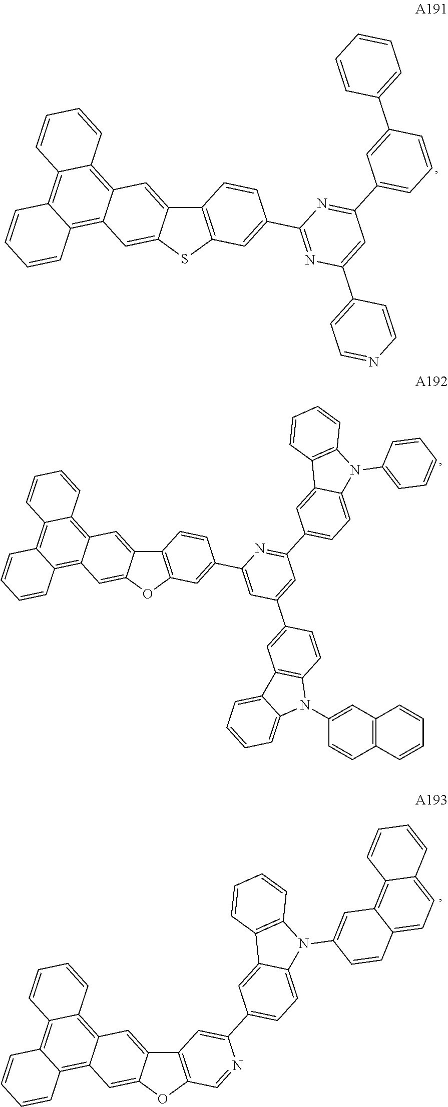 [SMILES: c1ccc(-c2cccc(-c3cc(-c4ccncc4)nc(-c4ccc5c(c4)sc4cc6c7ccccc7c7ccccc7c6cc45)n3)c2)cc1 ; c1ccc(-n2c3ccccc3c3cc(-c4cc(-c5ccc6c(c5)c5ccccc5n6-c5ccc6ccccc6c5)cc(-c5ccc6c(c5)oc5cc7c8ccccc8c8ccccc8c7cc56)n4)ccc32)cc1 ; c1ccc2c(c1)ccc1ccc(-n3c4ccccc4c4cc(-c5cc6c(cn5)oc5cc7c8ccccc8c8ccccc8c7cc56)ccc43)cc12]

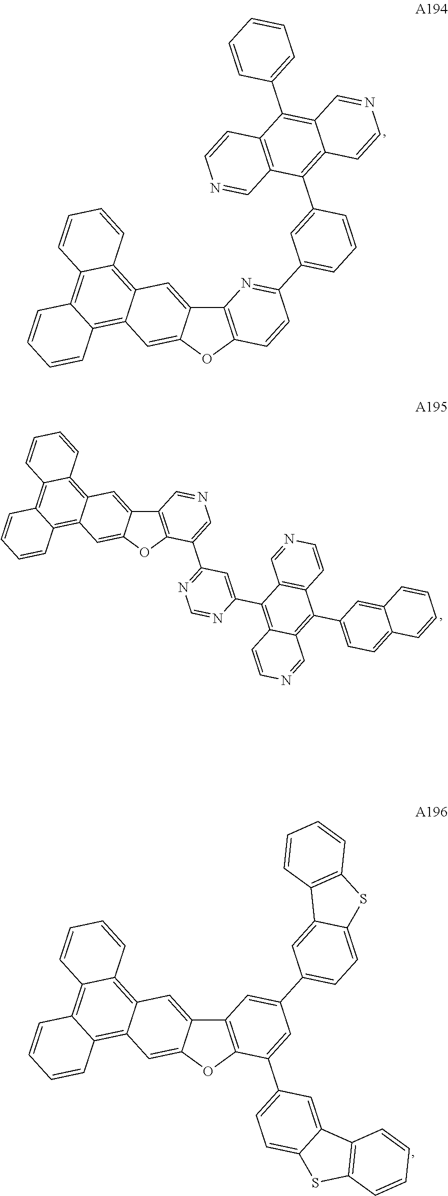 [SMILES: c1ccc(-c2c3ccncc3c(-c3cccc(-c4ccc5oc6cc7c8ccccc8c8ccccc8c7cc6c5n4)c3)c3ccncc23)cc1 ; c1ccc2cc(-c3c4cnccc4c(-c4cc(-c5cncc6c5oc5cc7c8ccccc8c8ccccc8c7cc56)ncn4)c4cnccc34)ccc2c1 ; c1ccc2c(c1)sc1ccc(-c3cc(-c4ccc5sc6ccccc6c5c4)c4oc5cc6c7ccccc7c7ccccc7c6cc5c4c3)cc12]

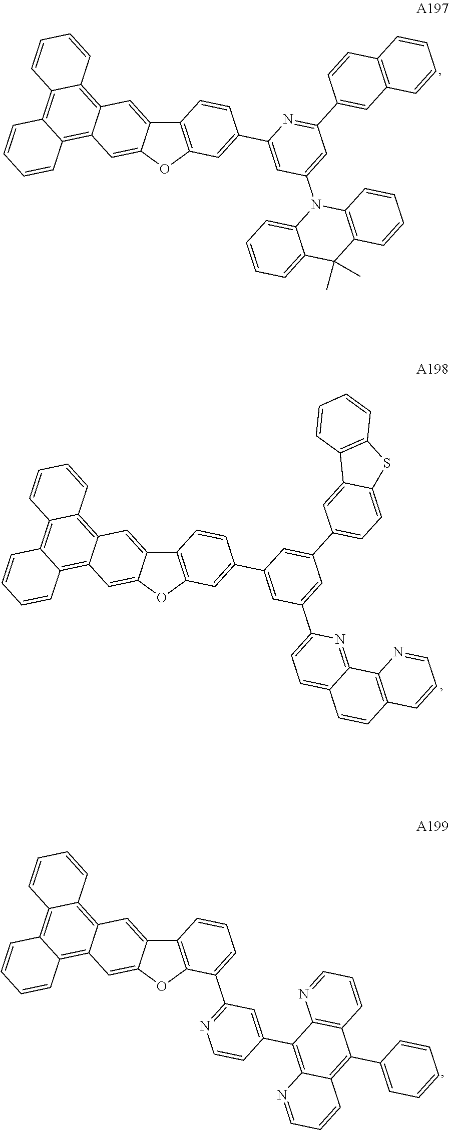 [SMILES: CC1(C)c2ccccc2N(c2cc(-c3ccc4ccccc4c3)nc(-c3ccc4c(c3)oc3cc5c6ccccc6c6ccccc6c5cc34)c2)c2ccccc21 ; c1cnc2c(c1)ccc1ccc(-c3cc(-c4ccc5c(c4)oc4cc6c7ccccc7c7ccccc7c6cc45)cc(-c4ccc5sc6ccccc6c5c4)c3)nc12 ; c1ccc(-c2c3cccnc3c(-c3ccnc(-c4cccc5c4oc4cc6c7ccccc7c7ccccc7c6cc45)c3)c3ncccc23)cc1]

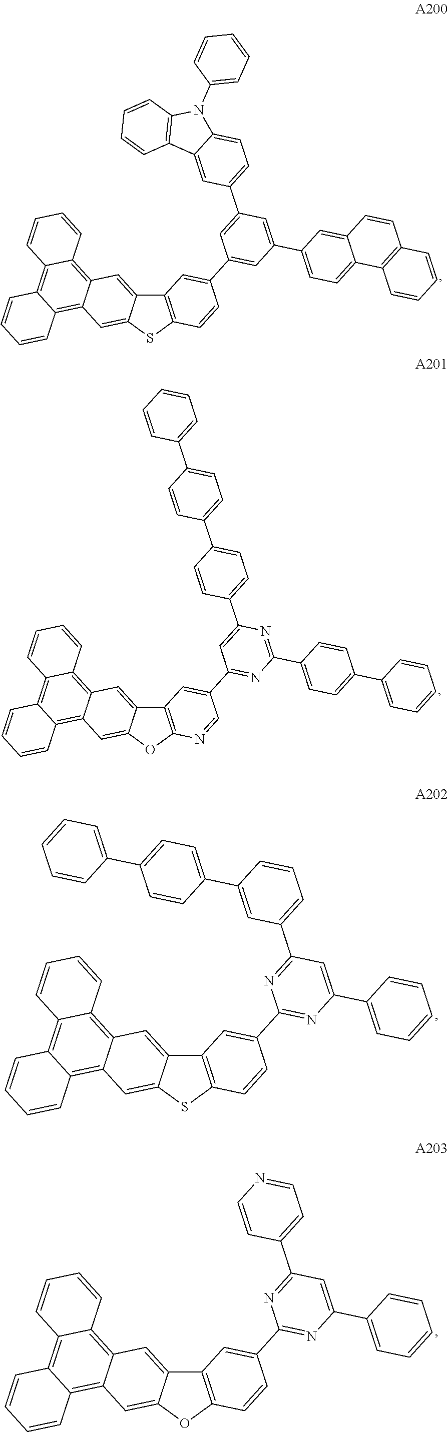 [SMILES: c1ccc(-n2c3ccccc3c3cc(-c4cc(-c5ccc6c(ccc7ccccc76)c5)cc(-c5ccc6sc7cc8c9ccccc9c9ccccc9c8cc7c6c5)c4)ccc32)cc1 ; c1ccc(-c2ccc(-c3ccc(-c4cc(-c5cnc6oc7cc8c9ccccc9c9ccccc9c8cc7c6c5)nc(-c5ccc(-c6ccccc6)cc5)n4)cc3)cc2)cc1 ; c1ccc(-c2ccc(-c3cccc(-c4cc(-c5ccccc5)nc(-c5ccc6sc7cc8c9ccccc9c9ccccc9c8cc7c6c5)n4)c3)cc2)cc1 ; c1ccc(-c2cc(-c3ccncc3)nc(-c3ccc4oc5cc6c7ccccc7c7ccccc7c6cc5c4c3)n2)cc1]

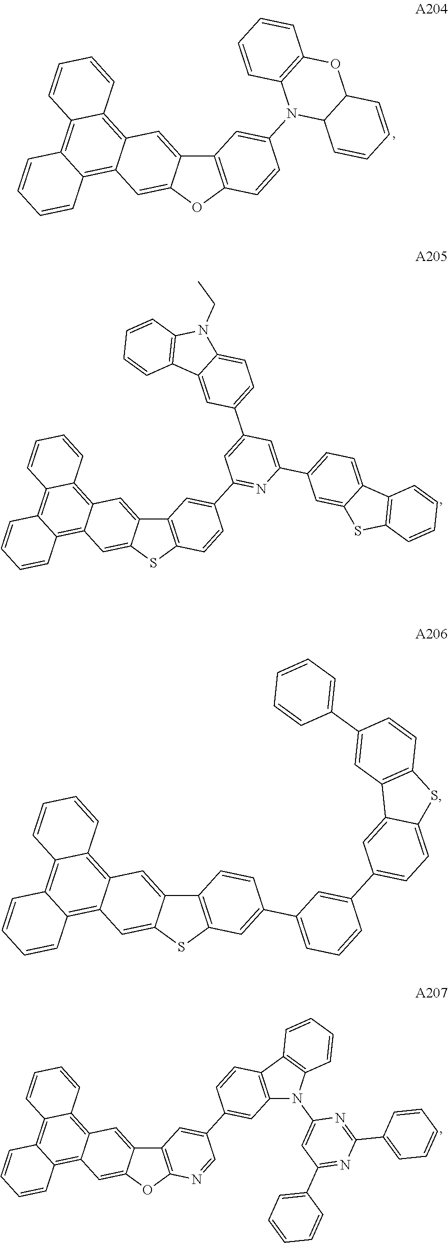 [SMILES: C1=CC2Oc3ccccc3N(c3ccc4oc5cc6c7ccccc7c7ccccc7c6cc5c4c3)C2C=C1 ; CCn1c2ccccc2c2cc(-c3cc(-c4ccc5c(c4)sc4ccccc45)nc(-c4ccc5sc6cc7c8ccccc8c8ccccc8c7cc6c5c4)c3)ccc21 ; c1ccc(-c2ccc3sc4ccc(-c5cccc(-c6ccc7c(c6)sc6cc8c9ccccc9c9ccccc9c8cc67)c5)cc4c3c2)cc1 ; c1ccc(-c2cc(-n3c4ccccc4c4ccc(-c5cnc6oc7cc8c9ccccc9c9ccccc9c8cc7c6c5)cc43)nc(-c3ccccc3)n2)cc1]

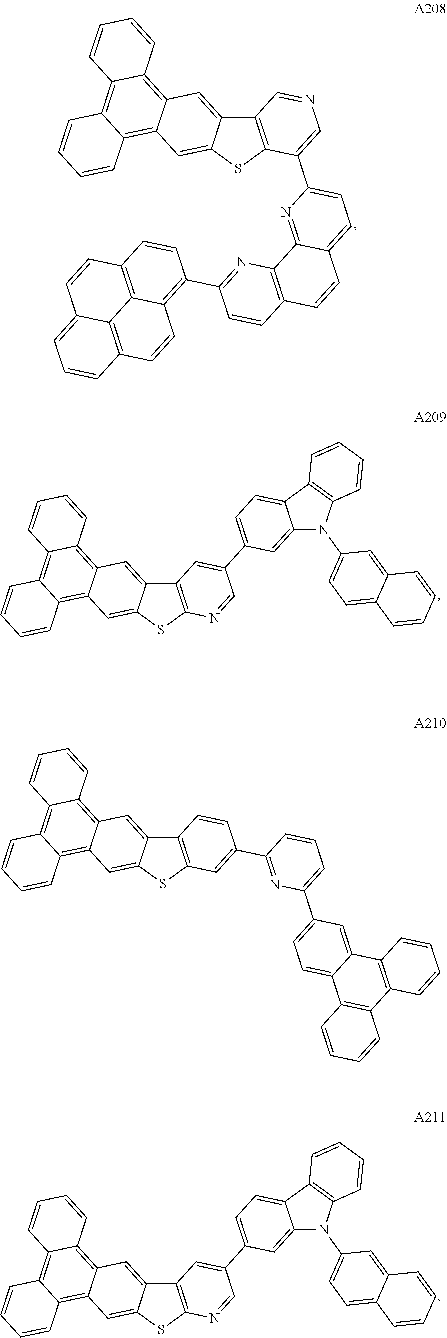 [SMILES: c1cc2ccc3ccc(-c4ccc5ccc6ccc(-c7cncc8c7sc7cc9c%10ccccc%10c%10ccccc%10c9cc78)nc6c5n4)c4ccc(c1)c2c34 ; c1ccc2cc(-n3c4ccccc4c4ccc(-c5cnc6sc7cc8c9ccccc9c9ccccc9c8cc7c6c5)cc43)ccc2c1 ; c1cc(-c2ccc3c(c2)sc2cc4c5ccccc5c5ccccc5c4cc23)nc(-c2ccc3c4ccccc4c4ccccc4c3c2)c1 ; c1ccc2cc(-n3c4ccccc4c4ccc(-c5cnc6sc7cc8c9ccccc9c9ccccc9c8cc7c6c5)cc43)ccc2c1]

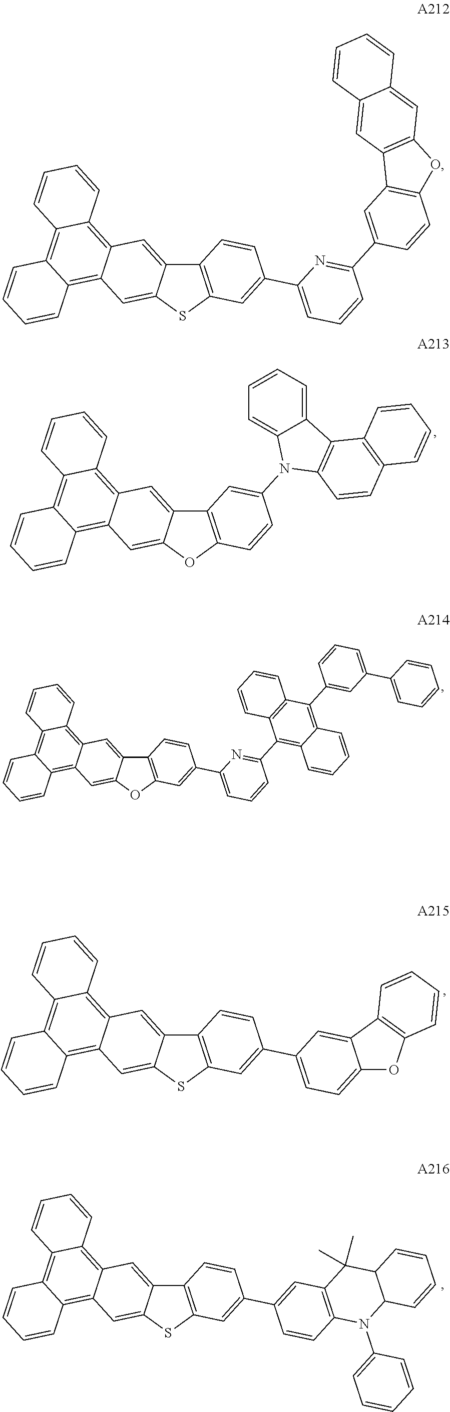 [SMILES: c1cc(-c2ccc3c(c2)sc2cc4c5ccccc5c5ccccc5c4cc23)nc(-c2ccc3oc4cc5ccccc5cc4c3c2)c1 ; c1ccc2c(c1)ccc1c2c2ccccc2n1-c1ccc2oc3cc4c5ccccc5c5ccccc5c4cc3c2c1 ; c1ccc(-c2cccc(-c3c4ccccc4c(-c4cccc(-c5ccc6c(c5)oc5cc7c8ccccc8c8ccccc8c7cc56)n4)c4ccccc34)c2)cc1 ; c1ccc2c(c1)oc1ccc(-c3ccc4c(c3)sc3cc5c6ccccc6c6ccccc6c5cc34)cc12 ; CC1(C)c2cc(-c3ccc4c(c3)sc3cc5c6ccccc6c6ccccc6c5cc34)ccc2N(c2ccccc2)C2C=CC=CC21]

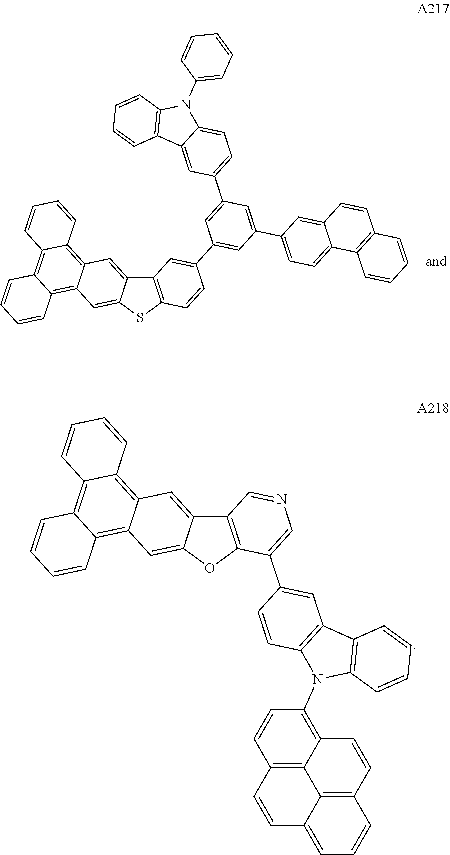 [SMILES: c1ccc(-n2c3ccccc3c3cc(-c4cc(-c5ccc6c(ccc7ccccc76)c5)cc(-c5ccc6sc7cc8c9ccccc9c9ccccc9c8cc7c6c5)c4)ccc32)cc1 ; c1cc2ccc3ccc(-n4c5ccccc5c5cc(-c6cncc7c6oc6cc8c9ccccc9c9ccccc9c8cc67)ccc54)c4ccc(c1)c2c34]

In another embodiment of the present invention, an organic electroluminescence device is disclosed. The organic electroluminescence device comprises a pair of electrodes having a cathode and an anode, and a light emitting layer and one or more organic thin film layers between the pair of electrodes. At least one of the light emitting layer and the organic thin film layer comprises the organic compound of formula (A).

In some embodiments, the light emitting layer comprising the organic compound of formula (A) is a phosphorescent host material. In certain embodiments, the light emitting layer comprising the organic compound of formula (A) is used as a hole blocking material or an electron transport material.

In a further embodiment of the present invention, the organic electroluminescence device is a lighting panel. In other embodiment of the present invention, the organic electroluminescence device is a backlight panel.

Detailed preparation of the organic compounds of the present invention will be clarified by exemplary embodiments below, but the present invention is not limited thereto.

EXAMPLE 1 show the preparation of Intermediates of the present invention, EXAMPLE 2 show the preparation of organic compounds of the present invention, and EXAMPLE 3 show the fabrication and test reports of the organic EL devices.

### Example 1

Synthesis of Intermediate 1a

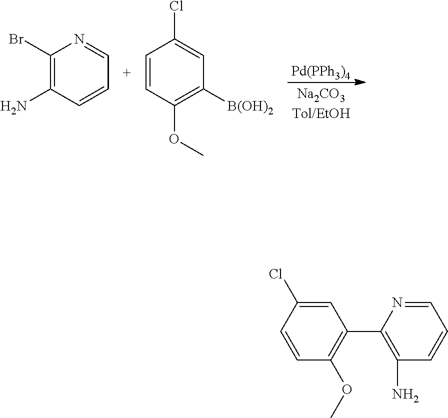

A mixture of 2-bromopyridin-3-amine (5.2 g, 30 mmol), 5-chloro-2-methoxyphenylboronic acid (6.2 g, 33 mmol), 30 ml of 2M Na₂CO_{3 (aq)}, 30 ml of ethanol and 60 ml of toluene was degassed and placed under nitrogen condition, and then Pd(PPh₃)₄ (0.34 g, 0.3 mmol) was added and heated at 100° C. for 12 hours. After the reaction was finished, the mixture was cooled to room temperature, and then extracted with ethyl acetate and water. The organic layer dried with anhydrous MgSO₄, and then evaporated under reduced pressure. The residue was purified by column chromatography on silica to give Intermediate 1a (4.5 g, 64%) as a white solid.

Synthesis of Intermediate 1b to 1f

Synthesis of Intermediate 1b to 1f was prepared according to the synthesis method of Intermediate 1a.

| | | | Weight |
|---|---|---|---|
| Reactant structure | | Product structure | Yield |
| 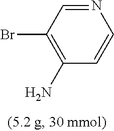 | 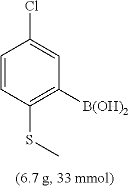 | 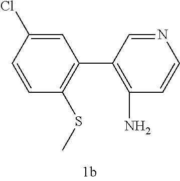 [SMILES: CSc1ccc(Cl)cc1-c1cnccc1N] | 4.7 g 62% |
| 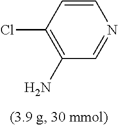 | 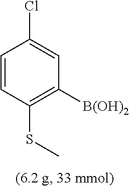 | 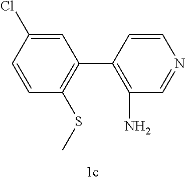 [SMILES: CSc1ccc(Cl)cc1-c1ccncc1N] | 4.5 g 60% |
| 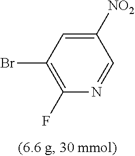 [SMILES: O=[N+]([O-])c1cnc(F)c(Br)c1] | 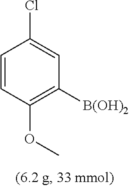 | 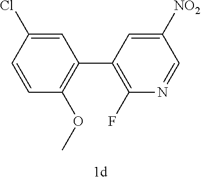 [SMILES: COc1ccc(Cl)cc1-c1cc([N+](=O)[O-])cnc1F] | 5.7 g 67% |
| 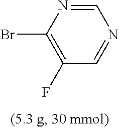 | 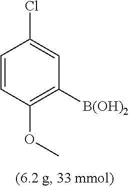 | 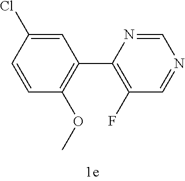 [SMILES: COc1ccc(Cl)cc1-c1ncncc1F] | 4.7 g 65% |
| 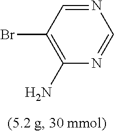 | 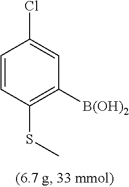 | 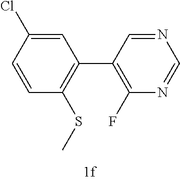 [SMILES: CSc1ccc(Cl)cc1-c1cncnc1F] | 5.0 g 66% |

Synthesis of Intermediate 2a

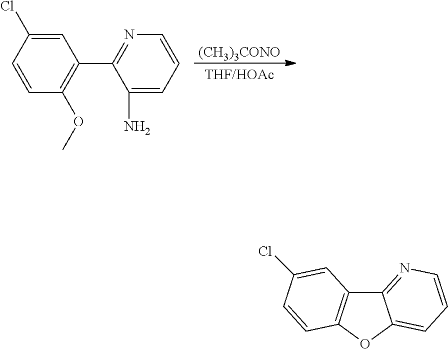

A mixture of Intermediate 1a (7 g, 30 mmol), 35 ml of tetrahydrofuran and 70 ml of glacial acetic acid (70 ml) was stirred at −10° C., and then tert-butyl nitrite (9.3 g, 8.9 ml, 90%, 90 mmol) was added over a period of 10 minutes. The reaction mixture was stirred at −10° C. for 2 hours, and then warmed to room temperature for 2 hours. The reaction was finished, and then diluted with 200 mL of water. The crude precipitate was purified by column chromatography on silica to afford Intermediate 2a (3.9 g, 64%) as a white solid.

Synthesis of Intermediate 2b and 2c

Synthesis of Intermediate 2b and 2c were according to the synthesis method of Intermediate 2a.

| | | Weight |
|---|---|---|
| Reactant structure | Product structure | Yield |
| 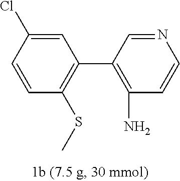 [SMILES: CSc1ccc(Cl)cc1-c1cnccc1N] | 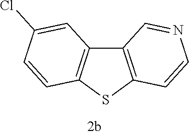 [SMILES: Clc1ccc2sc3ccncc3c2c1] | 4 g 60% |
| 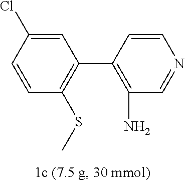 [SMILES: CSc1ccc(Cl)cc1-c1ccncc1N] | 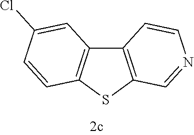 [SMILES: Clc1ccc2sc3cnccc3c2c1] | 3.9 g 59% |

Synthesis of Intermediate 3a

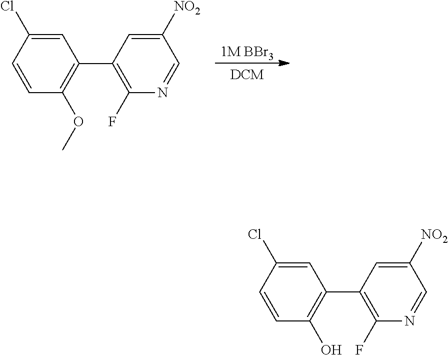

Under nitrogen condition, Intermediate 1d (11.3 g, 40 mmol) was dissolved in anhydrous dichloromethane (390 ml) at 0° C., 1 M BBr₃ (44 ml, 44 mmol) solution was added,

and then stirred for 2 hours. After the reaction was finished, 60 ml of methanol was added to the mixture, and then neutralized with 10% NaNCO_{3 (aq)}. The mixture was extracted with dichloromethane and water. The organic layer was separated and dried with anhydrous MgSO₄, and then the solvent was evaporated under reduced pressure. The residue was purified by column chromatography on silica to afford Intermediate 3a (8.5 g, 79%) as a white solid.

Synthesis of Intermediate 3b and 3c

Synthesis of Intermediate 3b and 3c were prepared according to the synthesis method of Intermediate 3a.

| | | Weight |
|---|---|---|
| Reactant structure | Product structure | Yield |
| 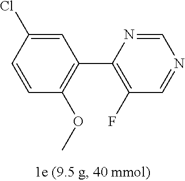 [SMILES: COc1ccc(Cl)cc1-c1ncncc1F] | 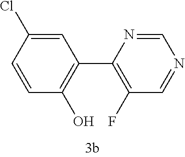 [SMILES: Oc1ccc(Cl)cc1-c1ncncc1F] | 6.8 g 76% |
| 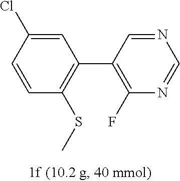 [SMILES: CSc1ccc(Cl)cc1-c1cncnc1F] | 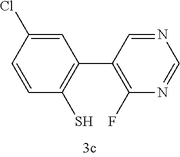 [SMILES: Fc1ncncc1-c1cc(Cl)ccc1S] | 7 g 73% |

Synthesis of Intermediate 4a

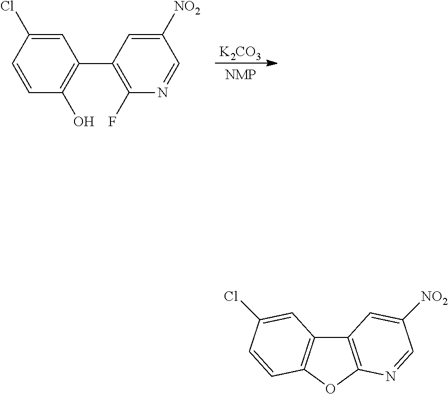

Under nitrogen condition, A mixture of Intermediate 3a (9.4 g, 30 mmol), K₂CO₃ (4.6 g, 33 mmol), and 85 ml of 1-methyl-2-pyrrolidone was added and heated at 150° C. for 16 hours. After the reaction was finished, the mixture was cooled to room temperature, and extracted with ethyl acetate and water. The organic layer was separated, and the solvent was evaporated under reduced pressure. The residue was purified by column chromatography on silica to give Intermediate 4a (6.4 g, 73%) as a white solid.

Synthesis of Intermediate 4b and 4c

Synthesis of Intermediate 4b and 4c were prepared according to the synthesis method of Intermediate 4a.

| | | Weight |
|---|---|---|
| Reactant structure | Product structure | Yield |
| 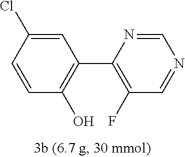 [SMILES: Oc1ccc(Cl)cc1-c1ncncc1F] | 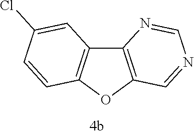 [SMILES: Clc1ccc2oc3cncnc3c2c1] | 5.7 g 76% |
| 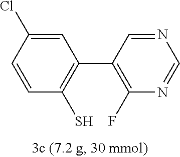 [SMILES: Fc1ncncc1-c1cc(Cl)ccc1S] | 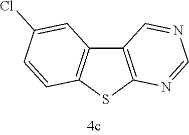 [SMILES: Clc1ccc2sc3ncncc3c2c1] | 4.9 g 74% |

Synthesis of Intermediate 5a

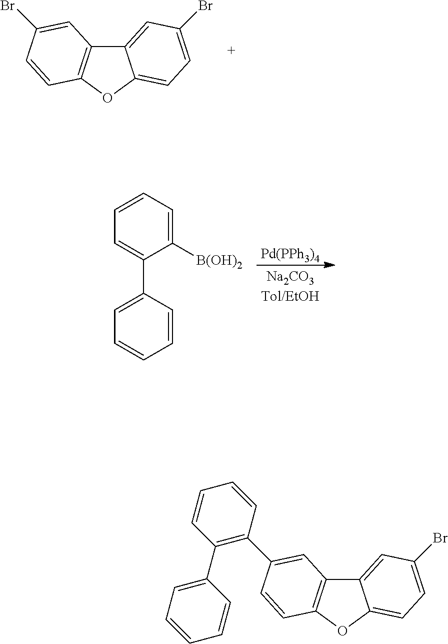 [SMILES: Brc1ccc2oc3ccc(Br)cc3c2c1 ; OB(O)c1ccccc1-c1ccccc1 ; Brc1ccc2oc3ccc(-c4ccccc4-c4ccccc4)cc3c2c1 ; "Pd(PPh3)4"]

A mixture of 2,8-dibromodibenzo[b,d]furan (16.3 g, 50 mmol), biphenyl-2-ylboronic acid (10.9 g, 55 mmol), 50 ml of 2M Na₂CO_{3 (aq)}, 50 ml of ethanol, 100 ml of toluene, and Pd(PPh₃)₄ (1.2 g, 1 mmol) was added under nitrogen condition, and then heated at 100° C. for 17 hours. After the reaction was finished, the mixture was cooled to room temperature. The organic layer was extracted with ethyl acetate and water, and then dried with anhydrous MgSO₄. The solvent was removed, and the residue was purified by column chromatography on silica to give Intermediate 5a (13.6 g, 68%) as a white solid.

Synthesis of Intermediate 5b to 5i

Synthesis of Intermediate 5b to 5i was prepared according to the synthesis method of Intermediate 5a.

| | | | Weight |
|---|---|---|---|
| Reactant structure | | Product structure | Yield |
| 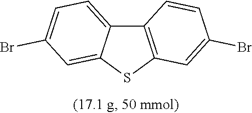 [SMILES: Brc1ccc2c(c1)sc1cc(Br)ccc12] | 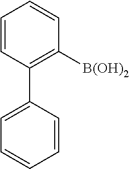 [SMILES: OB(O)c1ccccc1-c1ccccc1] | 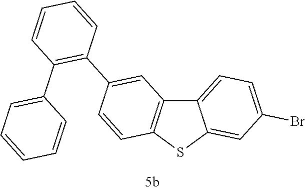 [SMILES: Brc1ccc2c(c1)sc1ccc(-c3ccccc3-c3ccccc3)cc12] | 13.1 g 63% |
| 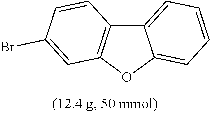 [SMILES: Brc1ccc2c(c1)oc1ccccc12] | 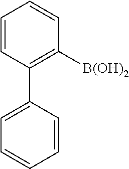 [SMILES: OB(O)c1ccccc1-c1ccccc1] | 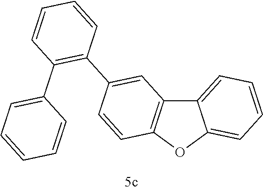 [SMILES: c1ccc(-c2ccccc2-c2ccc3oc4ccccc4c3c2)cc1] | 10.8 g 67% |
| 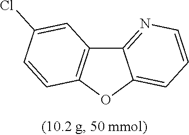 [SMILES: Clc1ccc2oc3cccnc3c2c1] | 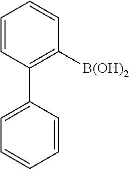 [SMILES: OB(O)c1ccccc1-c1ccccc1] | 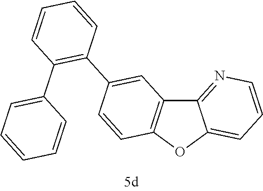 [SMILES: c1ccc(-c2ccccc2-c2ccc3oc4cccnc4c3c2)cc1] | 11.1 g 69% |
| 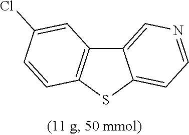 [SMILES: Clc1ccc2sc3ccncc3c2c1] | 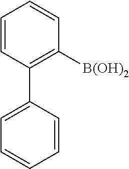 [SMILES: OB(O)c1ccccc1-c1ccccc1] | 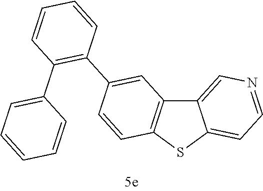 [SMILES: c1ccc(-c2ccccc2-c2ccc3sc4ccncc4c3c2)cc1] | 10.5 g 62% |
| 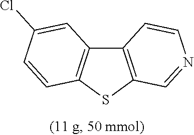 [SMILES: Clc1ccc2sc3cnccc3c2c1] | 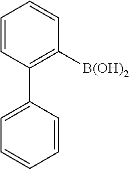 [SMILES: OB(O)c1ccccc1-c1ccccc1] | 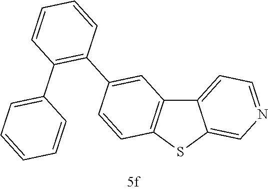 [SMILES: c1ccc(-c2ccccc2-c2ccc3sc4cnccc4c3c2)cc1] | 10.3 g 61% |
| 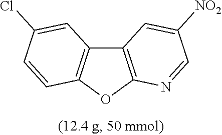 [SMILES: O=[N+]([O-])c1cnc2oc3ccc(Cl)cc3c2c1] | 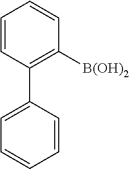 [SMILES: OB(O)c1ccccc1-c1ccccc1] | 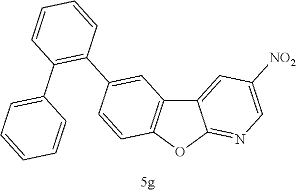 [SMILES: O=[N+]([O-])c1cnc2oc3ccc(-c4ccccc4-c4ccccc4)cc3c2c1] | 13.2 g 72% |
| 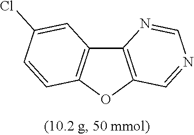 [SMILES: Clc1ccc2oc3cncnc3c2c1] | 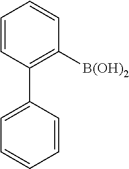 [SMILES: OB(O)c1ccccc1-c1ccccc1] | 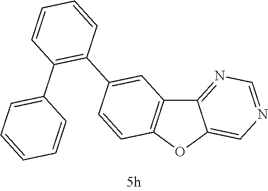 [SMILES: c1ccc(-c2ccccc2-c2ccc3oc4cncnc4c3c2)cc1] | 12.4 g 77% |
| 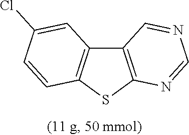 [SMILES: Clc1ccc2sc3ncncc3c2c1] | 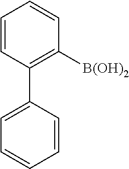 [SMILES: OB(O)c1ccccc1-c1ccccc1] | 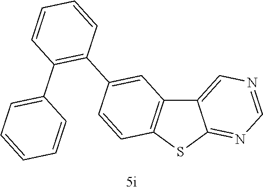 [SMILES: c1ccc(-c2ccccc2-c2ccc3sc4ncncc4c3c2)cc1] | 11.5 g 68% |

Synthesis of Intermediate 6a

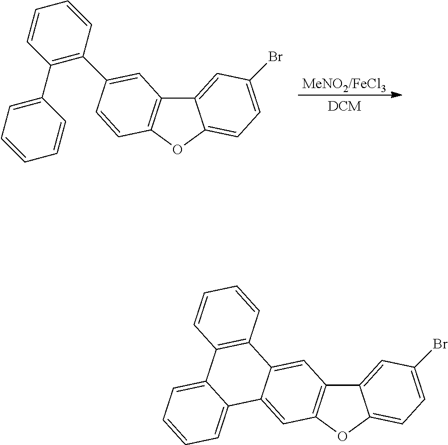

Intermediate 5a (24 g, 60 mmol) was dissolved in anhydrous dichloromethane (1200 ml) under nitrogen condition, and then the mixture of iron(III) chloride (48.7 g, 300 mmol) and 60 ml of nitromethane were added. The mixture was stirred for one hour, and then 500 ml of methanol was added to the mixture. The organic layer was separated and then dried with anhydrous MgSO₄. The solvent was evaporated under reduced pressure and then the residue was purified by column chromatography on silica to afford Intermediate 6a (13.8 g, 43%) as a white solid.

Synthesis of Intermediate 6b and 6i

Synthesis of Intermediate 6b to 6i was prepared according to the synthesis method of Intermediate 6a.

| | | Weight |
|---|---|---|
| Reactant structure | Product structure | Yield |
| 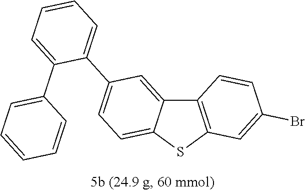 [SMILES: Brc1ccc2c(c1)sc1ccc(-c3ccccc3-c3ccccc3)cc12] | 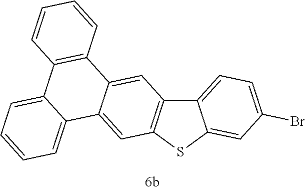 [SMILES: Brc1ccc2c(c1)sc1cc3c4ccccc4c4ccccc4c3cc12] | 11.2 g 45% |
| 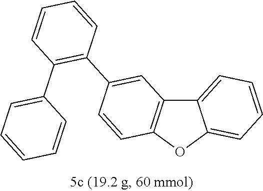 [SMILES: c1ccc(-c2ccccc2-c2ccc3oc4ccccc4c3c2)cc1] | 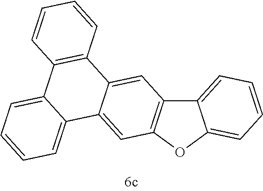 [SMILES: c1ccc2c(c1)oc1cc3c4ccccc4c4ccccc4c3cc12] | 11.8 g 62% |
| 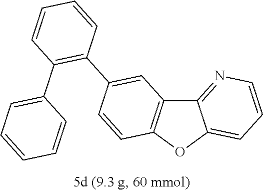 [SMILES: c1ccc(-c2ccccc2-c2ccc3oc4cccnc4c3c2)cc1] | 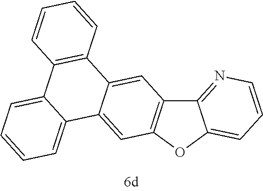 [SMILES: c1cnc2c(c1)oc1cc3c4ccccc4c4ccccc4c3cc12] | 11.3 g 59% |
| 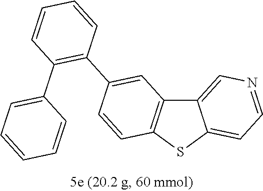 [SMILES: c1ccc(-c2ccccc2-c2ccc3sc4ccncc4c3c2)cc1] | 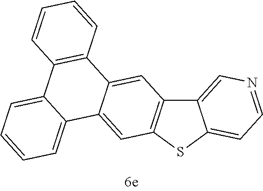 [SMILES: c1ccc2c(c1)c1ccccc1c1cc3c(cc21)sc1ccncc13] | 10.3 g 51% |
| 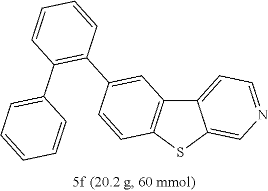 [SMILES: c1ccc(-c2ccccc2-c2ccc3sc4cnccc4c3c2)cc1] | 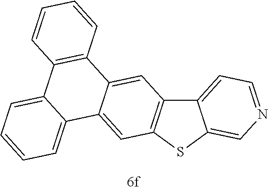 [SMILES: c1ccc2c(c1)c1ccccc1c1cc3c(cc21)sc1cnccc13] | 10.7 g 53% |
| 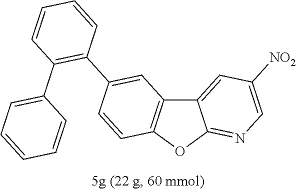 [SMILES: O=[N+]([O-])c1cnc2oc3ccc(-c4ccccc4-c4ccccc4)cc3c2c1] | 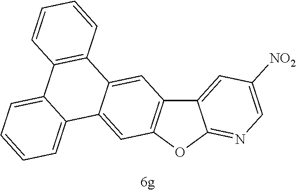 [SMILES: O=[N+]([O-])c1cnc2oc3cc4c5ccccc5c5ccccc5c4cc3c2c1] | 14.9 g 68% |
| 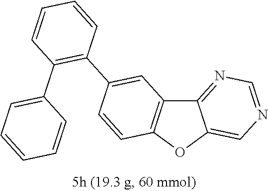 [SMILES: c1ccc(-c2ccccc2-c2ccc3oc4cncnc4c3c2)cc1] | 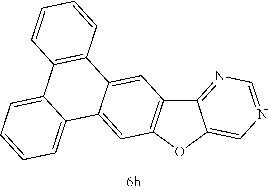 [SMILES: c1ccc2c(c1)c1ccccc1c1cc3c(cc21)oc1cncnc13] | 11.5 g 60% |
| 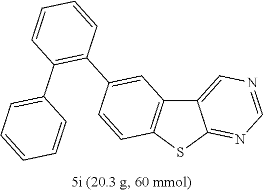 [SMILES: c1ccc(-c2ccccc2-c2ccc3sc4ncncc4c3c2)cc1] | 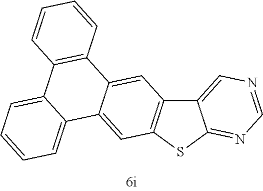 [SMILES: c1ccc2c(c1)c1ccccc1c1cc3c(cc21)sc1ncncc13] | 11.3 g 56% |

Synthesis of Intermediate 7

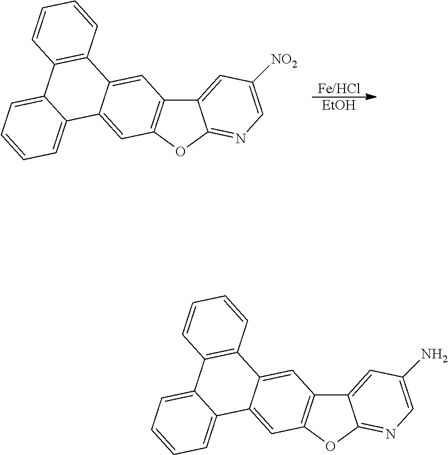 [SMILES: "EtOH" ; O=[N+]([O-])c1cnc2oc3cc4c5ccccc5c5ccccc5c4cc3c2c1 ; "Fe/HCl" ; Nc1cnc2oc3cc4c5ccccc5c5ccccc5c4cc3c2c1]

A mixture of Intermediate 6g (30 g, 82.3 mmol), iron powder (27.4 g, 493.8 mmol), 30 ml of conc. HCl and 400 ml of an aqueous ethanol (EtOH:H₂O=3:1) was added and then heated at 85° C. for 4 hours. After the reaction was finished, the mixture was cooled to room temperature. The mixture was filtered, and then the filtrate was extracted with ethyl acetate and water. The organic layer was dried with anhydrous MgSO₄ and the solvent was evaporated under reduced pressure. The residue was purified by column chromatography on silica to give Intermediate 7 (20.1 g, 73%) as a yellow solid.

Synthesis of Intermediate 8

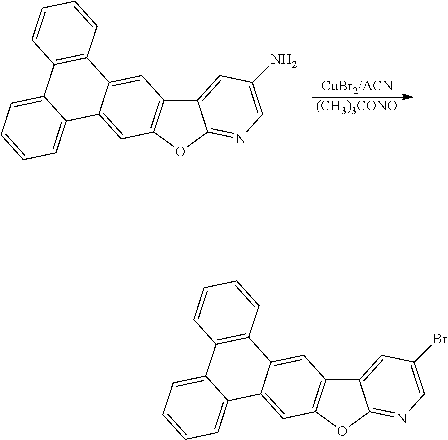

Under nitrogen condition, a mixture of tert-butyl nitrite (4.9 g, 4.8 ml, 90%, 48 mmol), anhydrous copper (II) bromide (8.9 g, 40 mmol) and 600 mL of anhydrous acetonitrile was heated at 80° C., and then Intermediate 7 (13.4 g, 40 mmol) was added slowly over a period of 1 hour, giving rise to a reaction with vigorous foaming and evolution of nitrogen gas. After the reaction was finished, the mixture was cooled to room temperature and poured into 600 ml of 10% HCl_{(aq)}. The crude precipitate was purified by column chromatography on silica to give Intermediate 8 (3.7 g, 23%) as a white solid.

Synthesis of Intermediate 9a to 9c

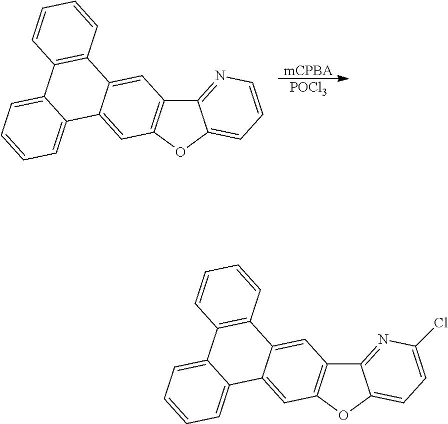

Under nitrogen condition, Intermediate 6i (16 g, 50 mmol) was dissolved in 480 ml of chloroform, mCPBA (11.2 g, 65 mmol) was added at 25° C., and then warmed to room temperature for 2 hours. After the reaction, the mixture was poured into 480 ml of 10% Na₂S₂O_{3 (aq)}, and then extracted with chloroform and water. The organic layer was dried with anhydrous Na₂SO₄ and the solvent was evaporated under reduced pressure. The crude material was purified by chromatography on silica gel to obtain an intermediate of N-oxide (10.9 g). Subsequently, 66 ml of phosphorus oxychloride was added to N-oxide (10.9 g), and heated to 95° C. for 10 hours. After the reaction was finished, the solvent was concentrated under reduced pressure, and then chloroform (200 ml) was added to the concentrate. The chloroform solution was added dropwise to sat. NaHCO_{3 (aq)}, and stirred for 1 hour. The mixture was extracted with chloroform, washed with sat. NaCl_{(aq)} and dried over Na₂SO₄. The organic layer was concentrated, the crude material was purified by chromatography on silica gel to give Intermediate 9a (7.3 g, 41%) as a white solid.

Synthesis of Intermediate 9b and 9c

Synthesis of Intermediate 9b and 9c were prepared according to the synthesis method of Intermediate 9a.

| | | Weight |
|---|---|---|
| Reactant structure | Product structure | Yield |
| 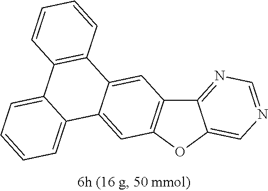 [SMILES: c1ccc2c(c1)c1ccccc1c1cc3c(cc21)oc1cncnc13] | 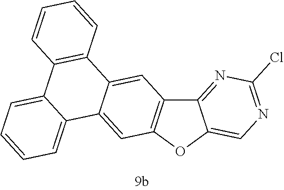 [SMILES: Clc1ncc2oc3cc4c5ccccc5c5ccccc5c4cc3c2n1] | 4.6 g 26% |
| 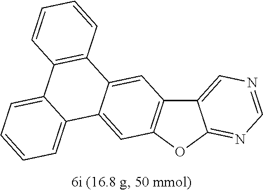 [SMILES: c1ccc2c(c1)c1ccccc1c1cc3c(cc21)oc1ncncc13] | 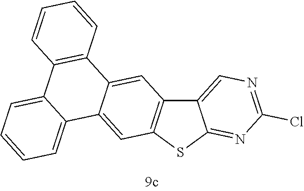 [SMILES: Clc1ncc2c(n1)sc1cc3c4ccccc4c4ccccc4c3cc12] | 4.5 g 24% |

Synthesis of Intermediate 10a

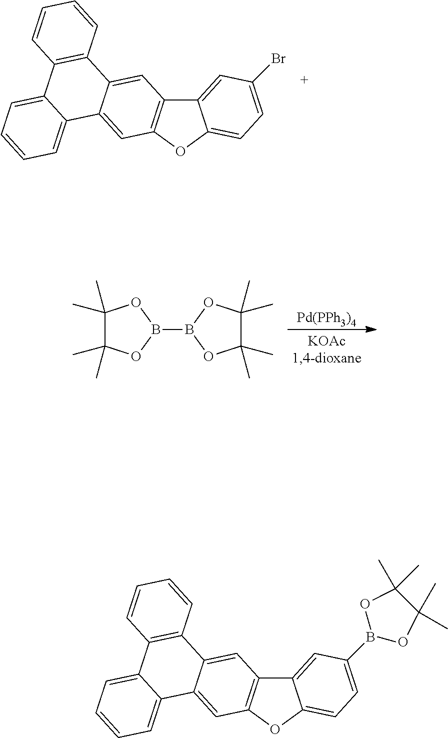

A mixture of Intermediate 6a (9.9 g, 25 mmol), bis(pinacolato)diboron (7.6 g, 30 mmol), potassium acetate (4.9 g, 50 mmol), 200 ml of 1,4-dioxane, and Pd(PPh₃)₄ (0.29 g, 0.25 mmol) was added, and then heated at 100° C. for 6 hours under nitrogen condition. After the mixture was cooled to room temperature, and then filtered to give the filterate. The filterate was evaporated under reduced pressure. The residue was purified by column chromatography on silica to give Intermediate 10a (7.9 g, 71%) as a white solid.

Synthesis of Intermediate 10b to 10f

Synthesis of Intermediate 10b to 10f was prepared according to the synthesis method of Intermediate 10a.

| | | Weight |
|---|---|---|
| Reactant structure | Product structure | Yield |
| 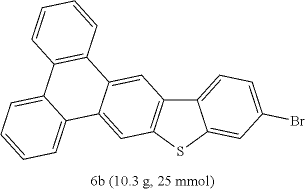 [SMILES: Brc1ccc2c(c1)sc1cc3c4ccccc4c4ccccc4c3cc12] | 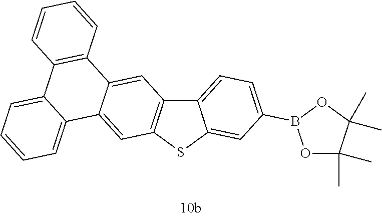 [SMILES: CC1(C)OB(c2ccc3c(c2)sc2cc4c5ccccc5c5ccccc5c4cc23)OC1(C)C] | 6.9 g 60% |
| 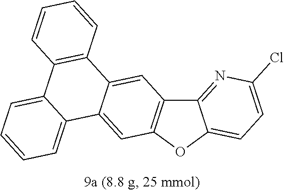 [SMILES: Clc1ccc2oc3cc4c5ccccc5c5ccccc5c4cc3c2n1] | 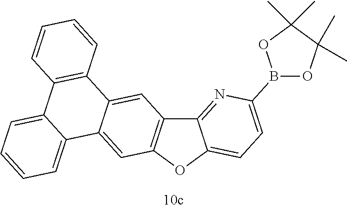 [SMILES: CC1(C)OB(c2ccc3oc4cc5c6ccccc6c6ccccc6c5cc4c3n2)OC1(C)C] | 6.6 g 59% |
| 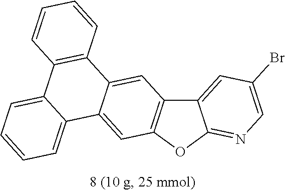 [SMILES: Brc1cnc2oc3cc4c5ccccc5c5ccccc5c4cc3c2c1] | 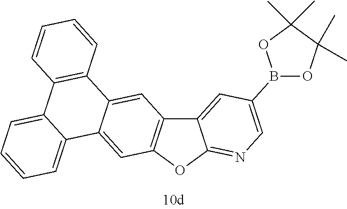 [SMILES: CC1(C)OB(c2cnc3oc4cc5c6ccccc6c6ccccc6c5cc4c3c2)OC1(C)C] | 6.8 g 61% |
| 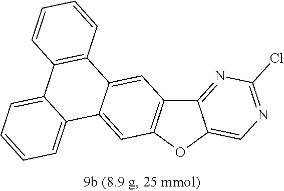 [SMILES: Clc1ncc2oc3cc4c5ccccc5c5ccccc5c4cc3c2n1] | 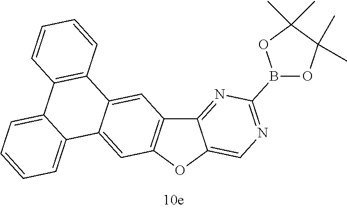 [SMILES: CC1(C)OB(c2ncc3oc4cc5c6ccccc6c6ccccc6c5cc4c3n2)OC1(C)C] | 7.0 g 63% |
| 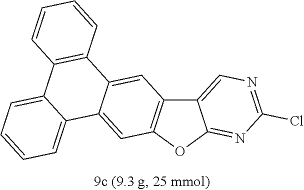 [SMILES: Clc1ncc2c(n1)oc1cc3c4ccccc4c4ccccc4c3cc12] | 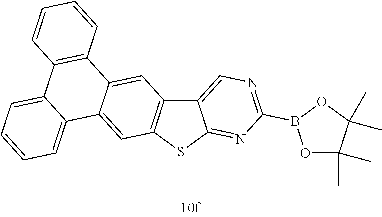 [SMILES: CC1(C)OB(c2ncc3c(n2)sc2cc4c5ccccc5c5ccccc5c4cc23)OC1(C)C] | 6.7 g 58% |

Synthesis of Intermediate 11a

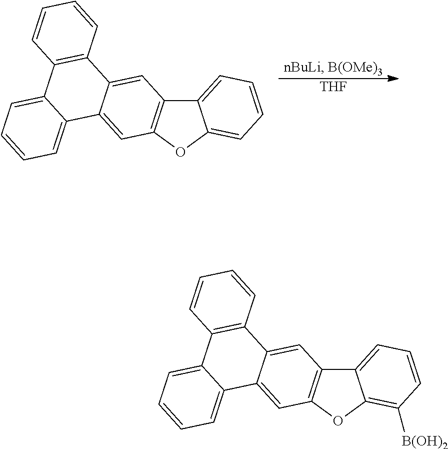

Intermediate 6c (19.1 g, 60 mmol) was dissolved in anhydrous tetrahydrofuran (380 ml) under nitrogen condition and then cooled to −68° C. 2.5M n-BuLi (30.2 ml, 72 mmol) was slowly dripped, and then heated to rt stirring for 2 hours under nitrogen condition. Cooled to −68° C. again and then trimethyl borate (9.4 g, 90 mmol) was slowly dripped, and then warmed to room temperature for 17 hours. Afterwards, 100 ml of 10% HCl_{(aq)} was added to the mixture and then the organic layer was separated, and the solvent was evaporated under reduced pressure. The residue was purified by column chromatography on silica to give Intermediate 11a (14.6 g, 67%) as a white solid.

Synthesis of Intermediate 11b to 10d

Synthesis of Intermediate 11b to 11d is prepared according to the synthesis method of Intermediate 11a.

| | | Yeild |
|---|---|---|
| Reactant structure | Product structure | (%) |
| 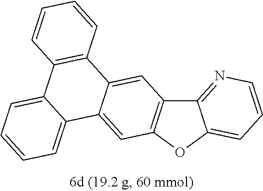 [SMILES: c1cnc2c(c1)oc1cc3c4ccccc4c4ccccc4c3cc12] | 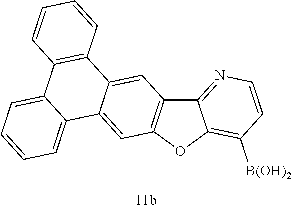 [SMILES: OB(O)c1ccnc2c1oc1cc3c4ccccc4c4ccccc4c3cc12] | 11.8 g 54% |
| 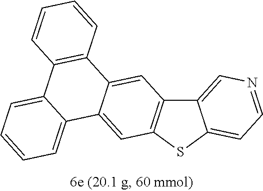 [SMILES: c1ccc2c(c1)c1ccccc1c1cc3c(cc21)sc1ccncc13] | 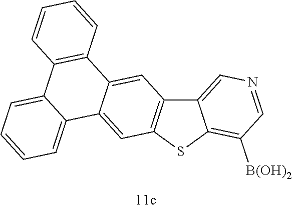 [SMILES: OB(O)c1cncc2c1sc1cc3c4ccccc4c4ccccc4c3cc12] | 11.6 g 51% |
| 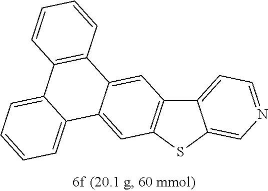 [SMILES: c1ccc2c(c1)c1ccccc1c1cc3c(cc21)sc1cnccc13] | 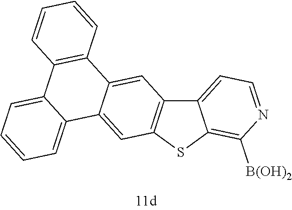 [SMILES: OB(O)c1nccc2c1sc1cc3c4ccccc4c4ccccc4c3cc12] | 10.9 g 48% |

Synthesis of Intermediate 12a

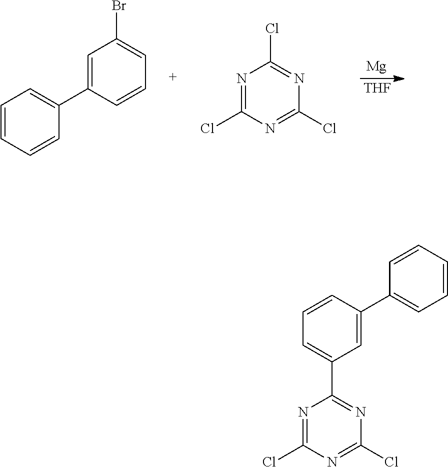

Under nitrogen condition, 2,4,6-trichloro-1,3,5-triazine (5.5 g, 30 mmol) was dissolved in anhydrous tetrahydrofuran (60 ml) at 0° C., and then Grignard Reagent was prepared from 3-bromobiphenyl (7 g, 30 mmol), magnesium powder (0.77 g, 31.5 mmol) and anhydrous THF (20 ml) was added and stirred at room temperature for 17 hours. Afterwards, 50 ml of 10% HCl_{(aq)} was added to the mixture and then the organic layer was separated, and the solvent was evaporated under reduced pressure. The residue was purified by column chromatography on silica to give Intermediate 12a (7.1 g, 78%) as a white solid.

Synthesis of Intermediate 12b and 12e

Synthesis of Intermediate 12b and 12e were prepared according to the synthesis method of Intermediate 12a.

| | | | Weight |
|---|---|---|---|
| Reactant structure | | Product structure | Yield |
| 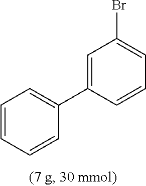 [SMILES: Brc1cccc(-c2ccccc2)c1] | 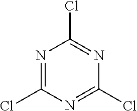 | 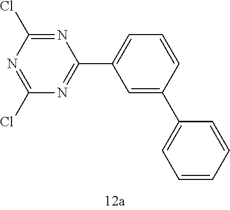 [SMILES: Clc1nc(Cl)nc(-c2cccc(-c3ccccc3)c2)n1] | 7.1 g 78% |
| 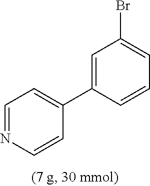 [SMILES: Brc1cccc(-c2ccncc2)c1] | 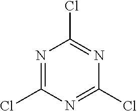 | 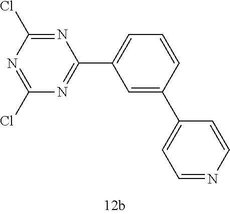 [SMILES: Clc1nc(Cl)nc(-c2cccc(-c3ccncc3)c2)n1] | 5.4 g 59% |
| 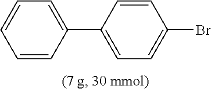 [SMILES: Brc1ccc(-c2ccccc2)cc1] | 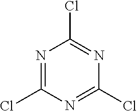 | 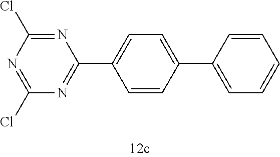 [SMILES: Clc1nc(Cl)nc(-c2ccc(-c3ccccc3)cc2)n1] | 7.4 82% |
| 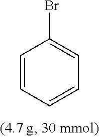 | 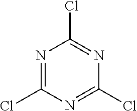 | 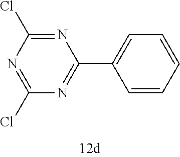 [SMILES: Clc1nc(Cl)nc(-c2ccccc2)n1] | 5.4 g 80% |
| 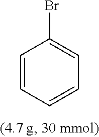 | 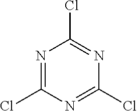 | 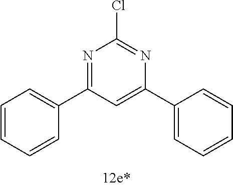 [SMILES: Clc1nc(-c2ccccc2)cc(-c2ccccc2)n1] | 3.1 g 39% |

*The reaction is heated at 65° C. for 17 hours.

Synthesis of Intermediate 13a

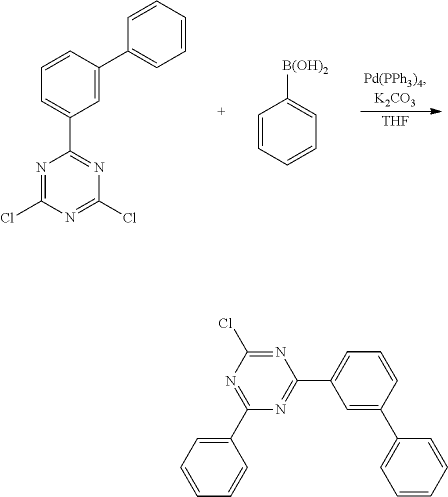

Under nitrogen condition, a mixture of Intermediate 12a (9.1 g, 30 mmol), phenylboronic acid (3.7 g, 30 mmol), 30 ml of 2M K₂CO_{3 (aq)}, 90 ml of tetrahydrofuran, and Pd(PPh₃)₄ (0.35 g, 0.3 mmol) was added under nitrogen condition, and then heated at 70° C. for 17 hours. After the reaction was finished, the mixture was extracted with ethyl acetate and water, and then the organic layer was evaporated under reduced pressure. The residue was purified by column chromatography on silica to give Intermediate 13a (5.4 g, 52%) as a white solid.

Synthesis of Intermediate 13b to 13h

Synthesis of Intermediate 13b to 13h was prepared according to the synthesis method of Intermediate 13a.

| | | | Weight |
|---|---|---|---|
| Reactant structure | | Product structure | Yield |
| 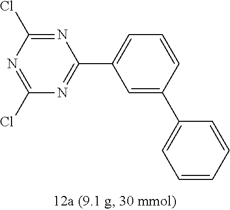 [SMILES: Clc1nc(Cl)nc(-c2cccc(-c3ccccc3)c2)n1] | 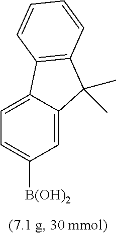 [SMILES: CC1(C)c2ccccc2-c2ccc(B(O)O)cc21] | 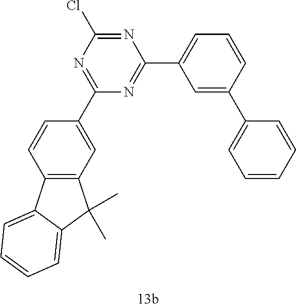 [SMILES: CC1(C)c2ccccc2-c2ccc(-c3nc(Cl)nc(-c4cccc(-c5ccccc5)c4)n3)cc21] | 7.2 g 52% |
| 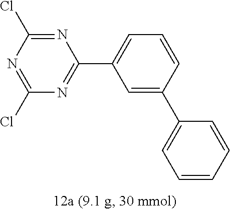 [SMILES: Clc1nc(Cl)nc(-c2cccc(-c3ccccc3)c2)n1] | 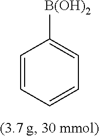 | 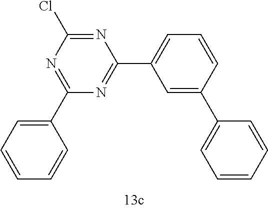 [SMILES: Clc1nc(-c2ccccc2)nc(-c2cccc(-c3ccccc3)c2)n1] | 6 g 58% |
| 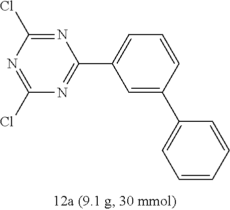 [SMILES: Clc1nc(Cl)nc(-c2cccc(-c3ccccc3)c2)n1] | 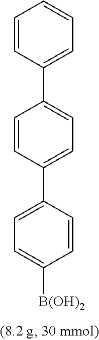 [SMILES: OB(O)c1ccc(-c2ccc(-c3ccccc3)cc2)cc1] | 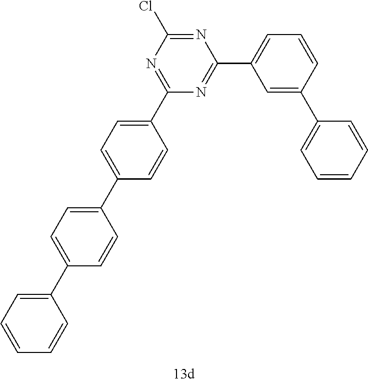 [SMILES: Clc1nc(-c2ccc(-c3ccc(-c4ccccc4)cc3)cc2)nc(-c2cccc(-c3ccccc3)c2)n1] | 6.4 g 43% |
| 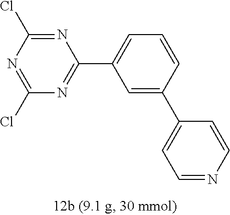 [SMILES: Clc1nc(Cl)nc(-c2cccc(-c3ccncc3)c2)n1] | 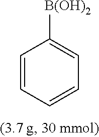 | 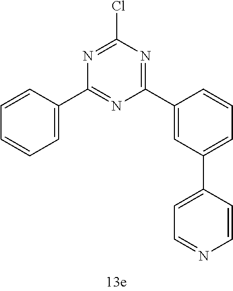 [SMILES: Clc1nc(-c2ccccc2)nc(-c2cccc(-c3ccncc3)c2)n1] | 7.5 g 54% |
| 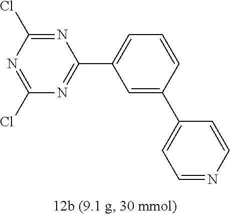 [SMILES: Clc1nc(Cl)nc(-c2cccc(-c3ccncc3)c2)n1] | 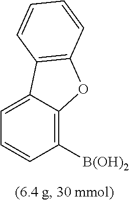 [SMILES: OB(O)c1cccc2c1oc1ccccc12] | 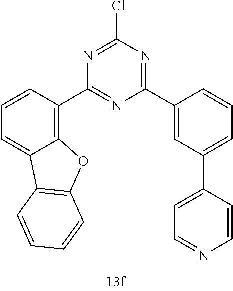 [SMILES: Clc1nc(-c2cccc(-c3ccncc3)c2)nc(-c2cccc3c2oc2ccccc23)n1] | 7.5 g 54% |
| 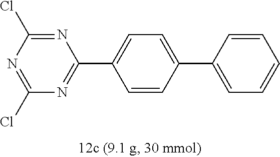 [SMILES: Clc1nc(Cl)nc(-c2ccc(-c3ccccc3)cc2)n1] | 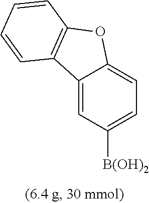 [SMILES: OB(O)c1ccc2oc3ccccc3c2c1] | 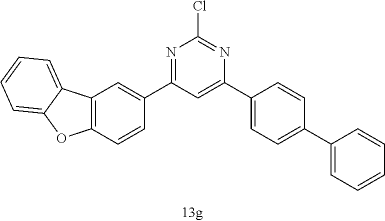 [SMILES: Clc1nc(-c2ccc(-c3ccccc3)cc2)cc(-c2ccc3oc4ccccc4c3c2)n1] | 6.6 g 51% |
| 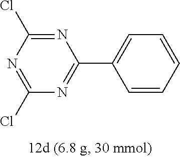 [SMILES: Clc1nc(Cl)nc(-c2ccccc2)n1] | 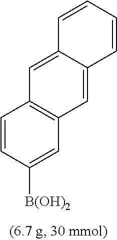 [SMILES: OB(O)c1ccc2cc3ccccc3cc2c1] | 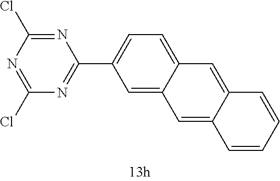 [SMILES: Clc1nc(Cl)nc(-c2ccc3cc4ccccc4cc3c2)n1] | 5.1 g 54% |

Synthesis of Intermediate 14

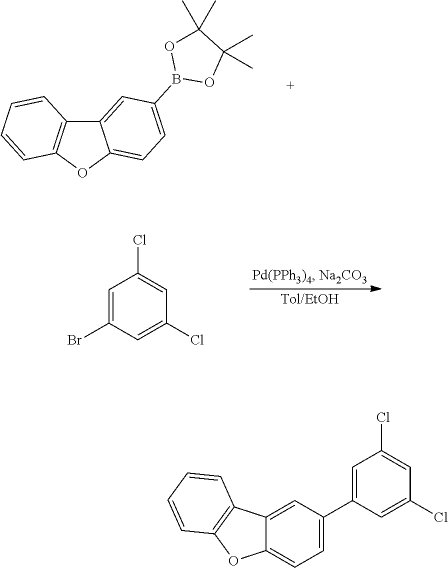

A mixture of 2-(dibenzo[b,d]furan-2-yl)-4,4,5,5-tetramethyl-1,3,2-dioxaborolane (17.6 g, 600 mmol), 1-bromo-3,5-dichlorobenzene (3.7 g, 30 mmol), 30 ml of 2M Na₂CO_{3 (aq)}, 45 ml of ethanol, 90 ml of toluene and Pd(PPh₃)₄ (0.35 g, 0.3 mmol) was added under nitrogen condition, and then heated at 100° C. for 17 hours. After the reaction was finished, the mixture was extracted with ethyl acetate and water, and then the organic layer was evaporated under reduced pressure. The residue was purified by column chromatography on silica to give Intermediate 14 (5.4 g, 52%) as a white solid.

Synthesis of Intermediate 15

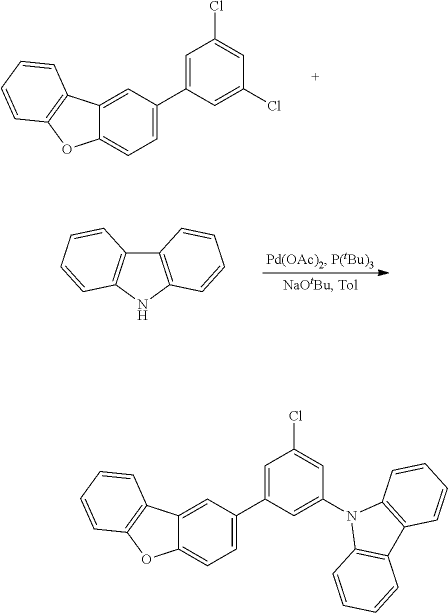

Under nitrogen condition, a mixture of Intermediate 14 (15.7 g, 50 mmol), carbazole (8.4 g, 50 mmol), NaOtBu (9.6 g, 100 mmol), 160 ml of toluene, and Pd(OAc)₂ (0.11 g, 0.5 mmol), and tri-tert-butylphosphine (0.2 g, 1 mmol) was added and then heated at 110° C. for 17 hours. After the reaction was finished and cooled to room temperature. The mixture was poured into 500 ml of methanol and then filtered to give a solid. The residue was purified by column chromatography on silica to give Intermediate 15 (7.9 g, 71%) as a white solid.

Synthesis of Intermediate 16a

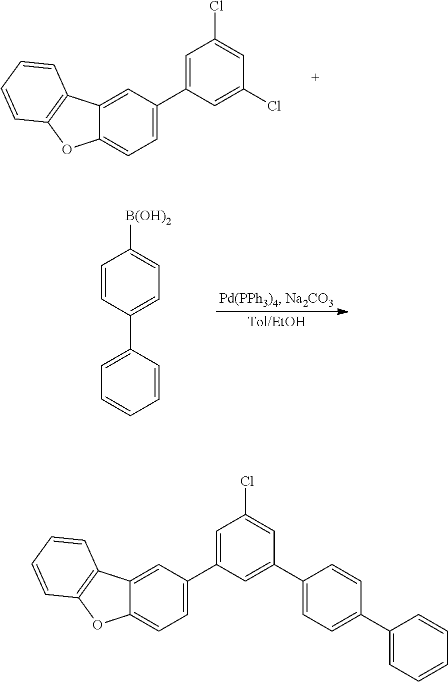

Under nitrogen condition, a mixture of Intermediate 14 (18.8 g, 60 mmol), biphenyl-4-ylboronic acid (11.9 g, 60 mmol), 60 ml of 2M Na₂CO_{3 (aq)}, 90 ml of ethanol, 180 ml of toluene, and Pd(PPh₃)₄ (0.69 g, 0.6 mmol) was added and then heated at 100° C. for 17 hours. After the reaction was finished, the mixture was extracted with ethyl acetate and water, and then the organic layer was evaporated under reduced pressure. The residue was purified by column chromatography on silica to give Intermediate 16a (11.1 g, 52%) as a white solid.

Synthesis of Intermediate 16b

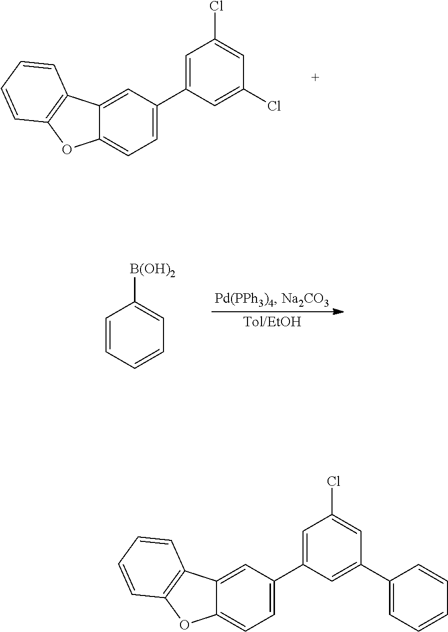

Synthesis of Compound 16b (14.7 g, 69%) was prepared from Intermediate 14 (18.8 g, 60 mmol) and phenylboronic acid (7.3 g, 60 mmol) according to the synthesis method of Compound 16a.

Synthesis of Intermediate 17a

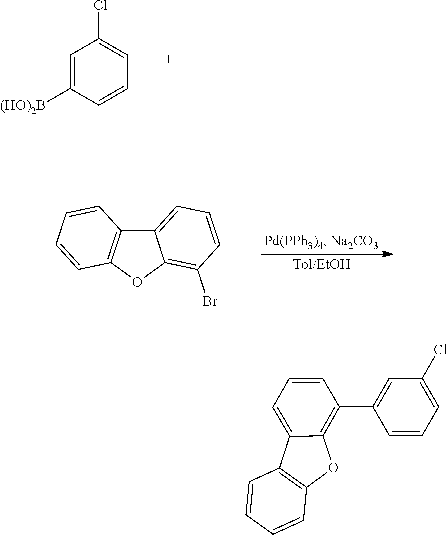

Under nitrogen condition, a mixture of 3-chlorophenylboronic acid (7.8 g, 60 mmol), 4-bromodibenzo[b,d]furan (16.3 g, 66 mmol), 60 ml of 2M Na₂CO_{3 (aq)}, 60 ml of ethanol, 120 ml of toluene, and Pd(PPh₃)₄ (0.69 g, 1.2 mmol) was added and then heated at 100° C. for 17 hours. After the reaction was finished, the mixture was extracted with ethyl acetate and water, and then the organic layer was evaporated under reduced pressure. The residue was purified by column chromatography on silica to give Intermediate 17a (12.9 g, 77%) as a white solid.

Synthesis of Intermediate 17b and 17c

Synthesis of Intermediate 17b and 17c were prepared according to the synthesis method of Intermediate 17a.

| | | | Weight |
|---|---|---|---|
| Reactant structure | | Product structure | Yield |
| 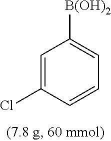 | 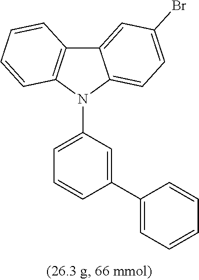 [SMILES: Brc1ccc2c(c1)c1ccccc1n2-c1cccc(-c2ccccc2)c1] | 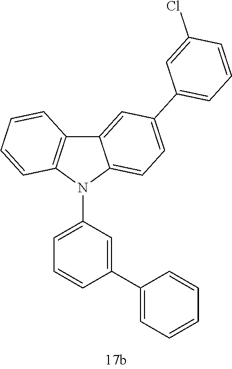 [SMILES: Clc1cccc(-c2ccc3c(c2)c2ccccc2n3-c2cccc(-c3ccccc3)c2)c1] | 15.7 g 61% |
| 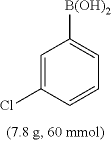 | 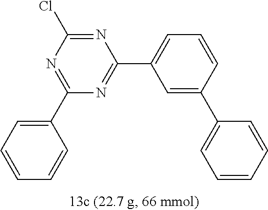 [SMILES: Clc1nc(-c2ccccc2)nc(-c2cccc(-c3ccccc3)c2)n1] | 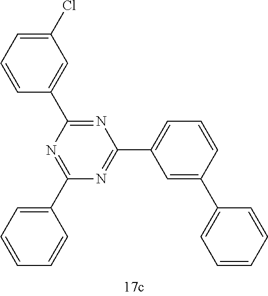 [SMILES: Clc1cccc(-c2nc(-c3ccccc3)nc(-c3cccc(-c4ccccc4)c3)n2)c1] | 15.6 g 62% |

### Example 2

Synthesis of Compound A4, A11, A13, A111, A139 and A160

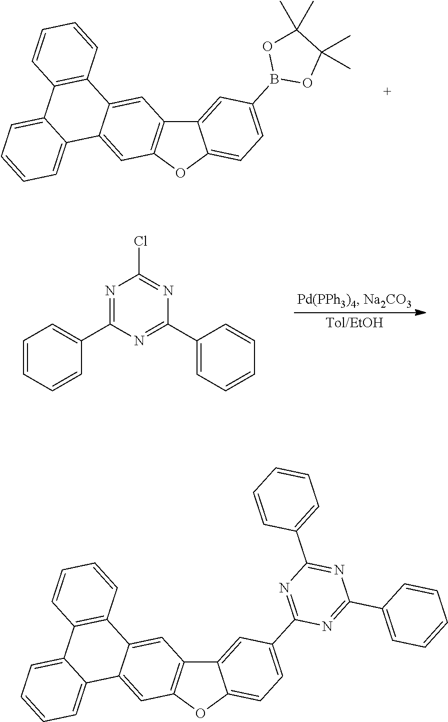

Under nitrogen condition, a mixture of Intermediate 10a (3.1 g, 7 mmol), 2-chloro-4,6-diphenyl-1,3,5-triazine (2.4 g, 9.1 mmol), 7 ml of 2M Na₂CO_{3 (aq)}, 7 ml of ethanol, 14 ml of toluene, Pd(PPh₃)₄ (0.16 g, 14 mmol) was added and heated at 100° C. for 17 hours. After the reaction was finished and cooled to room temperature. The mixture was filtered to give a solid. The solid was washed with water and methanol, and then filtered to give Compound A4 (2.7 g, 69%) as an off-white solid. MS (m/z, EI⁺): 549.63

Synthesis of Compound A11, A12, A13, A139 and A160

Synthesis of Compound A11, A12, A13, A139 and A160 were prepared according to the synthesis method of Compound A4.

| | | | Weight |
|---|---|---|---|
| Reactant structure | | Product structure | Yield |
| 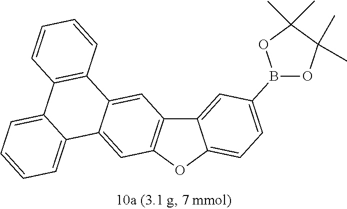 [SMILES: CC1(C)OB(c2ccc3oc4cc5c6ccccc6c6ccccc6c5cc4c3c2)OC1(C)C] | 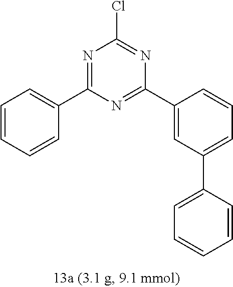 [SMILES: Clc1nc(-c2ccccc2)nc(-c2cccc(-c3ccccc3)c2)n1] | 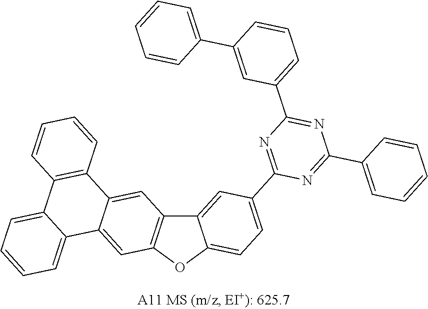 [SMILES: c1ccc(-c2cccc(-c3nc(-c4ccccc4)nc(-c4ccc5oc6cc7c8ccccc8c8ccccc8c7cc6c5c4)n3)c2)cc1] | 3.4 g 78% |
| 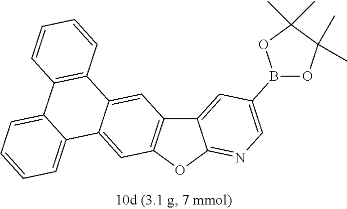 [SMILES: CC1(C)OB(c2cnc3oc4cc5c6ccccc6c6ccccc6c5cc4c3c2)OC1(C)C] | 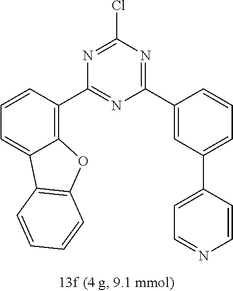 [SMILES: Clc1nc(-c2cccc(-c3ccncc3)c2)nc(-c2cccc3c2oc2ccccc23)n1] | 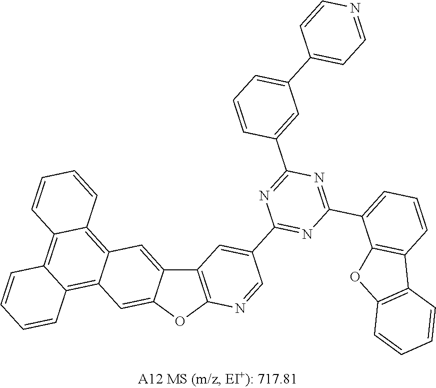 [SMILES: c1cc(-c2ccncc2)cc(-c2nc(-c3cnc4oc5cc6c7ccccc7c7ccccc7c6cc5c4c3)nc(-c3cccc4c3oc3ccccc34)n2)c1] | 3.8 g 76% |
| 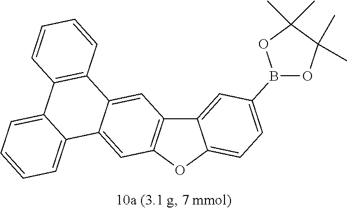 [SMILES: CC1(C)OB(c2ccc3oc4cc5c6ccccc6c6ccccc6c5cc4c3c2)OC1(C)C] | 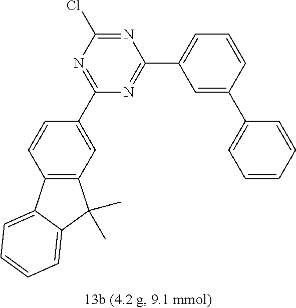 [SMILES: CC1(C)c2ccccc2-c2ccc(-c3nc(Cl)nc(-c4cccc(-c5ccccc5)c4)n3)cc21] | 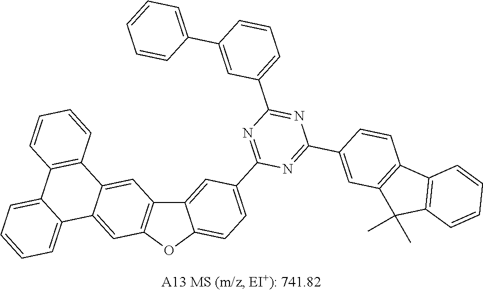 [SMILES: CC1(C)c2ccccc2-c2ccc(-c3nc(-c4cccc(-c5ccccc5)c4)nc(-c4ccc5oc6cc7c8ccccc8c8ccccc8c7cc6c5c4)n3)cc21] | 4.1 g 79% |
| 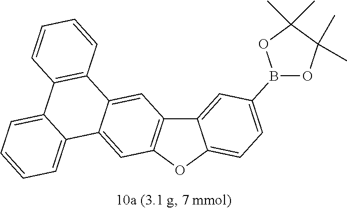 [SMILES: CC1(C)OB(c2ccc3oc4cc5c6ccccc6c6ccccc6c5cc4c3c2)OC1(C)C] | 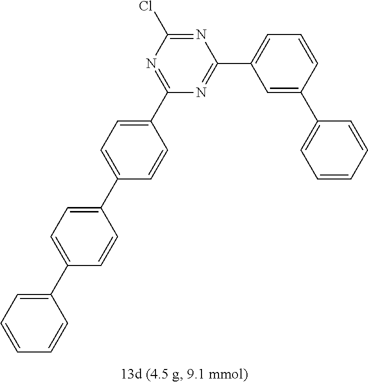 [SMILES: Clc1nc(-c2ccc(-c3ccc(-c4ccccc4)cc3)cc2)nc(-c2cccc(-c3ccccc3)c2)n1] | 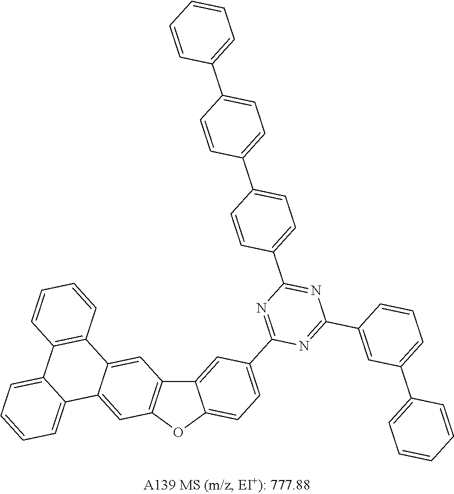 [SMILES: c1ccc(-c2ccc(-c3ccc(-c4nc(-c5cccc(-c6ccccc6)c5)nc(-c5ccc6oc7cc8c9ccccc9c9ccccc9c8cc7c6c5)n4)cc3)cc2)cc1] | 4.1 g 75% |
| 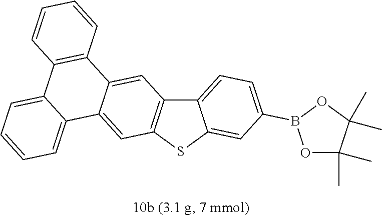 [SMILES: CC1(C)OB(c2ccc3c(c2)sc2cc4c5ccccc5c5ccccc5c4cc23)OC1(C)C] | 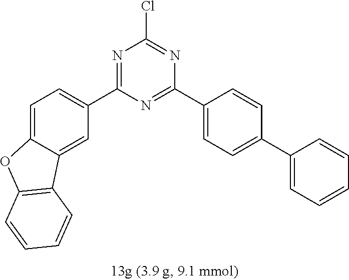 [SMILES: Clc1nc(-c2ccc(-c3ccccc3)cc2)nc(-c2ccc3oc4ccccc4c3c2)n1] | 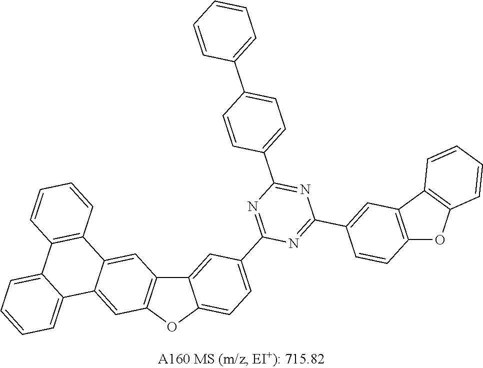 [SMILES: c1ccc(-c2ccc(-c3nc(-c4ccc5oc6ccccc6c5c4)nc(-c4ccc5oc6cc7c8ccccc8c8ccccc8c7cc6c5c4)n3)cc2)cc1] | 3.9 g 78% |

Synthesis of Compound A27

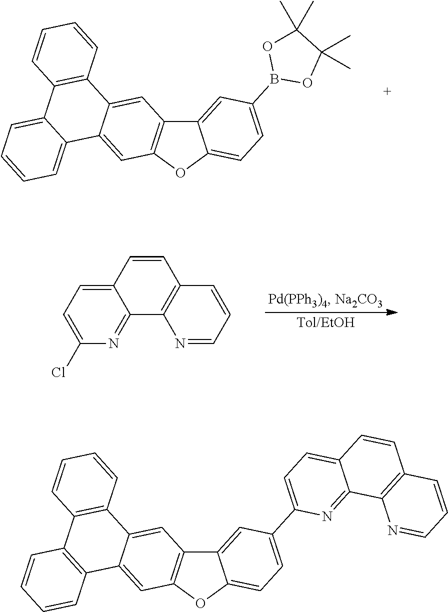

Synthesis of Compound A27 (2.4 g, 69%) was prepared from Intermediate 10a (2.8 g, 8 mmol) and 2-chloro-1,10-phenanthroline (1.9 g, 8.8 mmol) according to the synthesis method of Compound A4. MS (m/z, EI⁺): 496.55

Synthesis of Compound A72

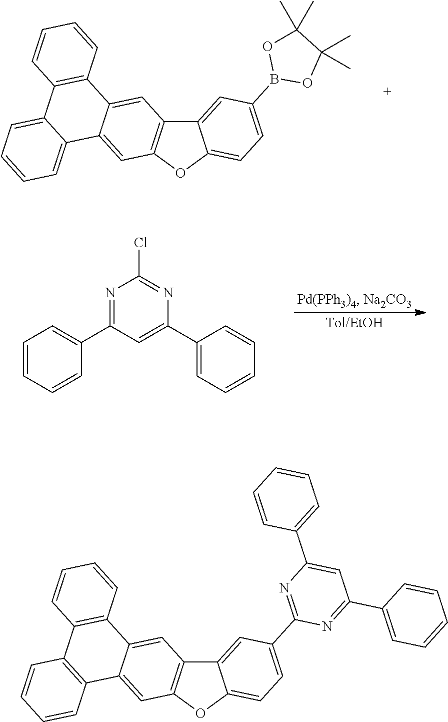

Synthesis of Compound A72 (2.6 g, 68%) was prepared from Intermediate 10a (2.8 g, 8 mmol) and 2-chloro-4,6-diphenylpyrimidine (2.6 g, 9.6 mmol) according to the synthesis method of Compound A4. MS (m/z, EI⁺): 548.59

Synthesis of Compound A7 and A167

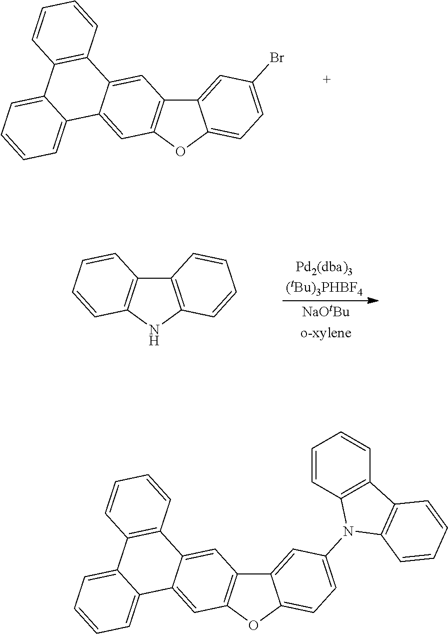

Under nitrogen condition, a mixture of Intermediate 6a (3.2 g, 8 mmol), carbazol (1.5 g, 8.8 mmol), NaOtBu (1.5 g, 16 mmol), 60 ml of o-xylene, tri-tert-butylphosphonium-tetrafluoroborate (0.09 g, 0.32 mmol) and Pd₂(dba)₃ (0.15 g, 0.16 mmol) was added and then heated at 130° C. for 17 hours. After the reaction was finished and cooled to room temperature. The mixture was filtered to give a solid. The solid was washed with water and MeOH, and then filtered to give Compound A7 (2.7 g, 69%) as an off-white solid. MS (m/z, EI⁺): 483.51

Synthesis of Compound A167 (3.9 g (77%) was prepared from Intermediate 6a (2.8 g, 8 mmol) and 3,6-diphenyl-9H-carbazole (2.8 g, 8.8 mmol) according to the synthesis method of Compound A7. MS (m/z, EI⁺): 635.78

Synthesis of Compound A35,

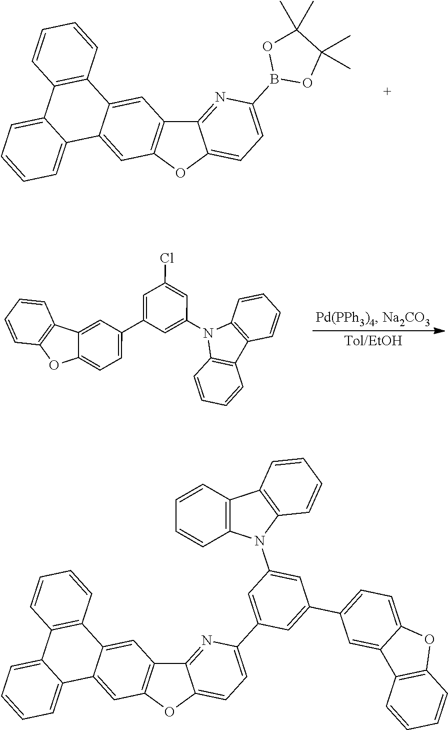

Under nitrogen condition, a mixture of Intermediate 10c (4 g, 9 mmol), Intermediate 15 (4 g, 9 mmol), 9 ml of 2M Na₂CO_{3 (aq)}, 9 ml of ethanol, 18 ml of toluene, Pd(PPh₃)₄ (0.21 g, 0.18 mmol) was added and heated at 100° C. for 17 hours. After the reaction was finished and cooled to room temperature. The mixture was filtered to give a solid. The solid was washed with water and methanol, and then filtered to give Compound A35 (5.2 g, 79%) as an off-white solid.

Synthesis of Compound A79, A143, A168 and A188

Synthesis of Compound A79, A143, A168 and A188 were prepared according to the synthesis method of Compound A35.

| | | | Weight |
|---|---|---|---|
| Reactant structure | | Product structure | Yield |
| 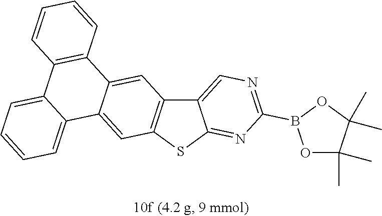 [SMILES: CC1(C)OB(c2ncc3c(n2)sc2cc4c5ccccc5c5ccccc5c4cc23)OC1(C)C] | 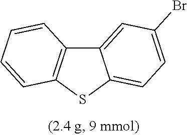 [SMILES: Brc1ccc2sc3ccccc3c2c1] | 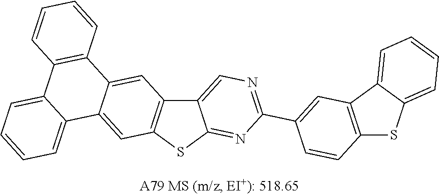 [SMILES: c1ccc2c(c1)sc1ccc(-c3ncc4c(n3)sc3cc5c6ccccc6c6ccccc6c5cc34)cc12] | 2.9 g 63% |
| 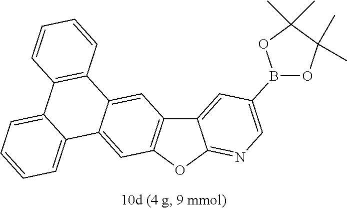 [SMILES: CC1(C)OB(c2cnc3oc4cc5c6ccccc6c6ccccc6c5cc4c3c2)OC1(C)C] | 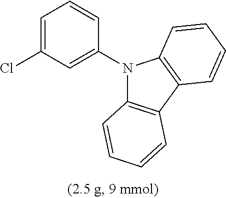 [SMILES: Clc1cccc(-n2c3ccccc3c3ccccc32)c1] | 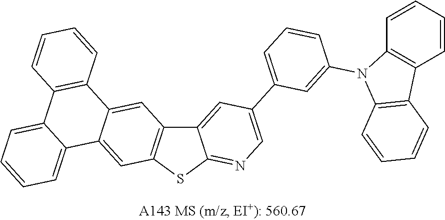 [SMILES: c1cc(-c2cnc3sc4cc5c6ccccc6c6ccccc6c5cc4c3c2)cc(-n2c3ccccc3c3ccccc32)c1] | 3.5 g 70% |
| 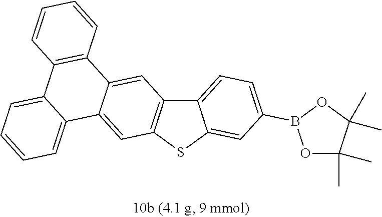 [SMILES: CC1(C)OB(c2ccc3c(c2)sc2cc4c5ccccc5c5ccccc5c4cc23)OC1(C)C] | 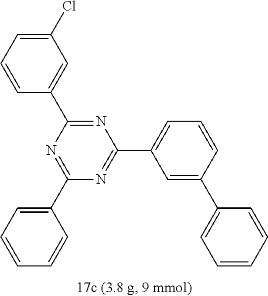 [SMILES: Clc1cccc(-c2nc(-c3ccccc3)nc(-c3cccc(-c4ccccc4)c3)n2)c1] | 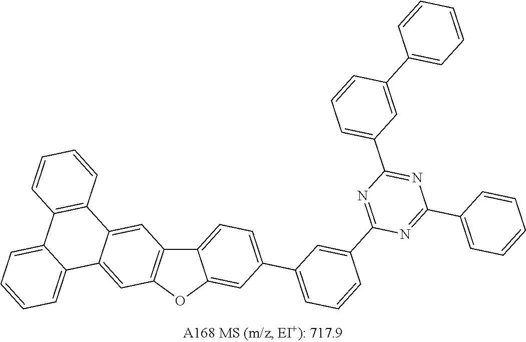 [SMILES: c1ccc(-c2cccc(-c3nc(-c4ccccc4)nc(-c4cccc(-c5ccc6c(c5)oc5cc7c8ccccc8c8ccccc8c7cc56)c4)n3)c2)cc1] | 5 g 78% |
| 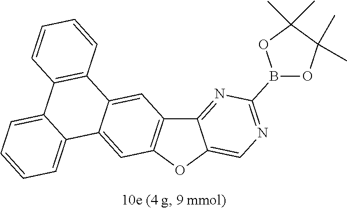 [SMILES: CC1(C)OB(c2ncc3oc4cc5c6ccccc6c6ccccc6c5cc4c3n2)OC1(C)C] | 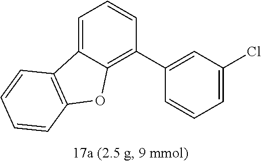 [SMILES: Clc1cccc(-c2cccc3c2oc2ccccc23)c1] | 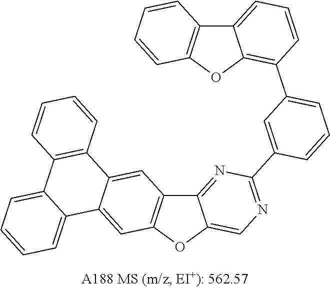 [SMILES: c1cc(-c2ncc3oc4cc5c6ccccc6c6ccccc6c5cc4c3n2)cc(-c2cccc3c2oc2ccccc23)c1] | 3.5 g 69% |

Synthesis of Compound A80

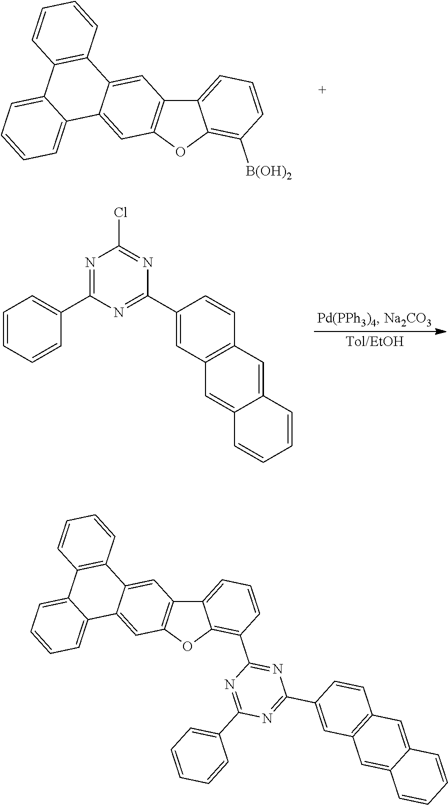

Under nitrogen condition, a mixture of Intermediate 11a (3.3 g, 8 mmol), Intermediate 13h (3.2 g, 8.8 mmol), 8 ml of 2M Na₂CO_{3 (aq)}, 8 ml of ethanol, 16 ml of toluene, Pd(PPh₃)₄ (0.18 g, 0.16 mmol) was added and heated at 100° C. for 17 hours. After the reaction was finished and cooled to room temperature. The mixture was filtered to give a solid. The solid was washed with water and methanol, and then filtered to give Compound A80 (4.1 g, 79%) as an off-white solid. MS (m/z, EI⁺): 649.69

Synthesis of Compound A83, A110, A162, A171 and A182

Synthesis of Compound A83, A110, A162, A171 and A182 were prepared according to the synthesis method of Compound A80.

| | | | Weight |
|---|---|---|---|
| Reactant structure | | Product structure | Yield |
| 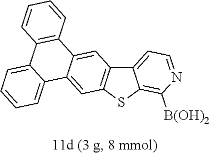 [SMILES: OB(O)c1nccc2c1sc1cc3c4ccccc4c4ccccc4c3cc12] | 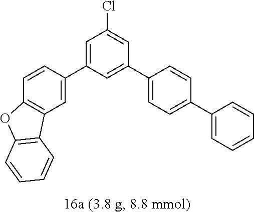 [SMILES: Clc1cc(-c2ccc(-c3ccccc3)cc2)cc(-c2ccc3oc4ccccc4c3c2)c1] | 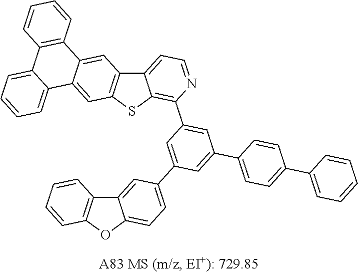 [SMILES: c1ccc(-c2ccc(-c3cc(-c4ccc5oc6ccccc6c5c4)cc(-c4nccc5c4sc4cc6c7ccccc7c7ccccc7c6cc45)c3)cc2)cc1] | 4.6 g 78% |
| 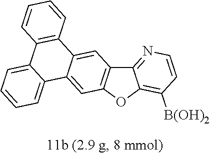 [SMILES: OB(O)c1ccnc2c1oc1cc3c4ccccc4c4ccccc4c3cc12] | 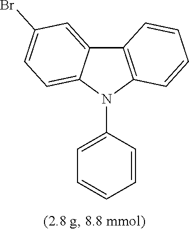 [SMILES: Brc1ccc2c(c1)c1ccccc1n2-c1ccccc1] | 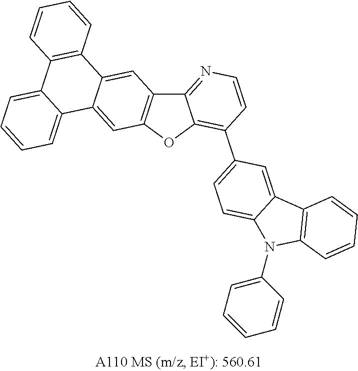 [SMILES: c1ccc(-n2c3ccccc3c3cc(-c4ccnc5c4oc4cc6c7ccccc7c7ccccc7c6cc45)ccc32)cc1] | 3.4 g 76% |
| 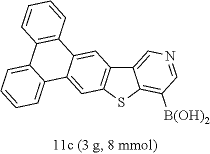 [SMILES: OB(O)c1cncc2c1sc1cc3c4ccccc4c4ccccc4c3cc12] | 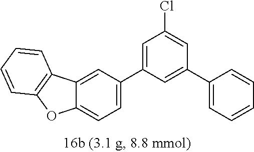 [SMILES: Clc1cc(-c2ccccc2)cc(-c2ccc3oc4ccccc4c3c2)c1] | 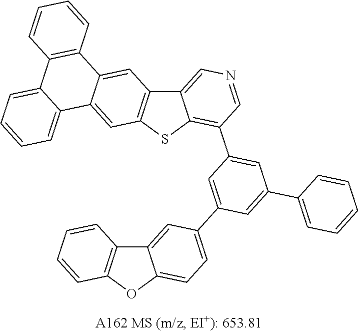 [SMILES: c1ccc(-c2cc(-c3ccc4oc5ccccc5c4c3)cc(-c3cncc4c3sc3cc5c6ccccc6c6ccccc6c5cc34)c2)cc1] | 4.1 g 79% |
| 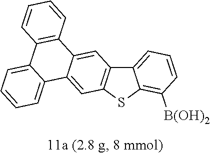 [SMILES: OB(O)c1cccc2c1sc1cc3c4ccccc4c4ccccc4c3cc12] | 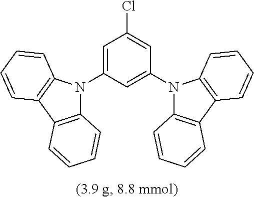 [SMILES: Clc1cc(-n2c3ccccc3c3ccccc32)cc(-n2c3ccccc3c3ccccc32)c1] | 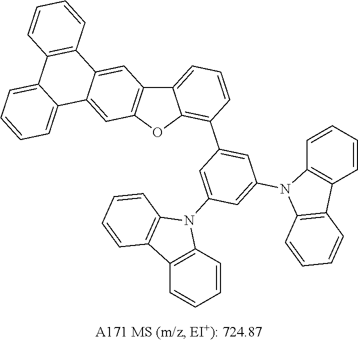 [SMILES: c1cc(-c2cc(-n3c4ccccc4c4ccccc43)cc(-n3c4ccccc4c4ccccc43)c2)c2oc3cc4c5ccccc5c5ccccc5c4cc3c2c1] | 4.4 g 76% |
| 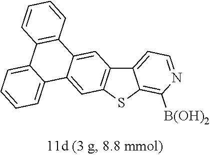 [SMILES: OB(O)c1nccc2c1sc1cc3c4ccccc4c4ccccc4c3cc12] | 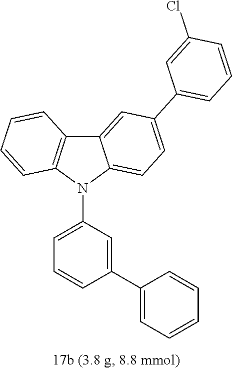 [SMILES: Clc1cccc(-c2ccc3c(c2)c2ccccc2n3-c2cccc(-c3ccccc3)c2)c1] | 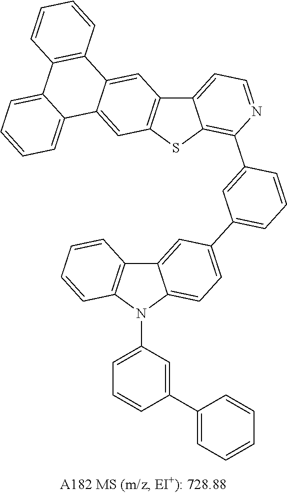 [SMILES: c1ccc(-c2cccc(-n3c4ccccc4c4cc(-c5cccc(-c6nccc7c6sc6cc8c9ccccc9c9ccccc9c8cc67)c5)ccc43)c2)cc1] | 4.4 g 76% |

### General Method of Producing Organic EL Device

ITO-coated glasses with 9-12 ohm/square in resistance and 120-160 nm in thickness are provided (hereinafter ITO substrate) and cleaned in a number of cleaning steps in an ultrasonic bath (e.g. detergent, deionized water). Before vapor deposition of the organic layers, cleaned ITO substrates are further treated by UV and ozone. All pre-treatment processes for ITO substrate are under clean room (class 100).

The organic layers are applied onto the ITO substrate in order by vapor deposition in a high-vacuum unit (10⁻⁷ Torr), such as: resistively heated quartz boats. The thickness of the respective layer and the vapor deposition rate (0.1-0.3 nm/sec) are precisely monitored or set with the aid of a quartz-crystal monitor. It is also possible, as described above, for individual layers to consist of more than one compound, e.g. a host material doped with a dopant material in the light emitting layer. This is successfully achieved by co-vaporization from two or more sources, which means organic compounds of the present invention is thermally stable.

Dipyrazino[2,3-f:2,3-]quinoxaline-2,3,6,7,10,11-hexacarbonitrile (HAT-CN) is used to form the hole injection layer, and N,N-bis(naphthalene-1-yl)-N,N-bis(phenyl)-benzidine (NPB) is used to form the hole transporting layer of the organic EL device. N-(biphenyl-4-yl)-9,9-dimethyl-N-(4′-phenyl-biphen-yl-4-yl)-9H-fluoren-2-amine (EB2) is used to form the electron blocking layer. For emitting device, H2 is used as blue emitting host and (E)-6-(4-(diphenylamino)styryl)-N,N-diphenylnaphthalen-2-amine (D1) is used as blue dopant. HB3 is used as hole blocking material (HBM), and 2-(10,10-dimethyl-10H-indeno[2,1-b]triphenylen-12-yl)-4,6-diphenyl-1,3,5-triazine (ET2) is used as electron transporting material to co-deposit with 8-hydroxyquinolato-lithium (LiQ) in organic EL devices. H2 and H3 are used as phosphorescent host for phosphorescent system, Ir(2-phq)₂(acac) and Ir(piq)₂(acac) are used as phosphorescent dopant of light emitting layer for comparison in the device test. The chemical structure of producing standard organic OLED device control and comparable material in this invention are shown as following:

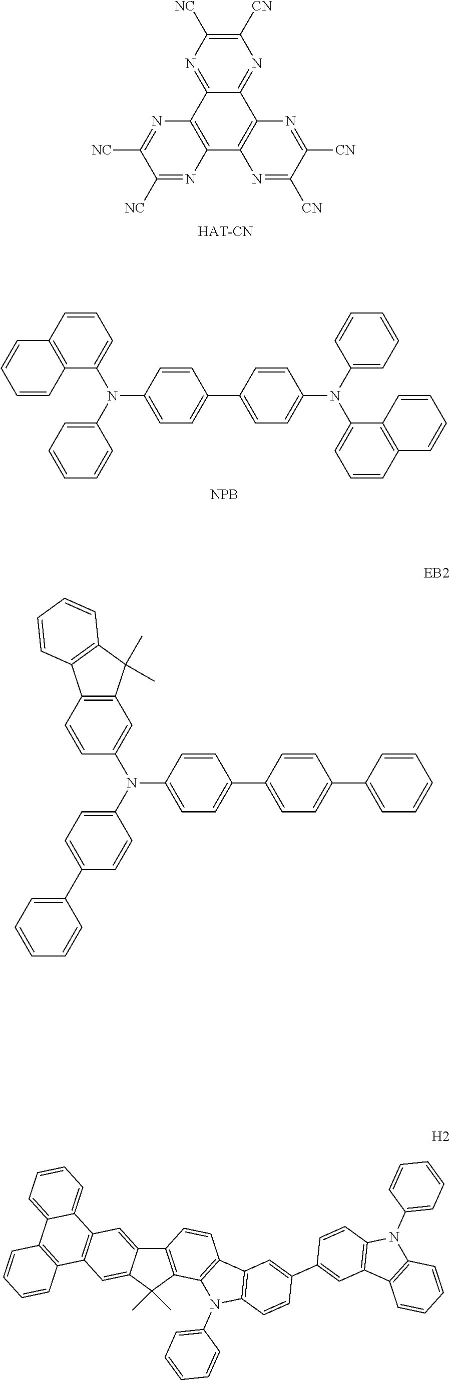 [SMILES: N#Cc1nc2c3nc(C#N)c(C#N)nc3c3nc(C#N)c(C#N)nc3c2nc1C#N ; c1ccc(N(c2ccc(-c3ccc(N(c4ccccc4)c4cccc5ccccc45)cc3)cc2)c2cccc3ccccc23)cc1 ; "NPB" ; CC1(C)c2ccccc2-c2ccc(N(c3ccc(-c4ccccc4)cc3)c3ccc(-c4ccc(-c5ccccc5)cc4)cc3)cc21 ; CC1(C)c2cc3c4ccccc4c4ccccc4c3cc2-c2ccc3c4cc(-c5ccc6c(c5)c5ccccc5n6-c5ccccc5)ccc4n(-c4ccccc4)c3c21]

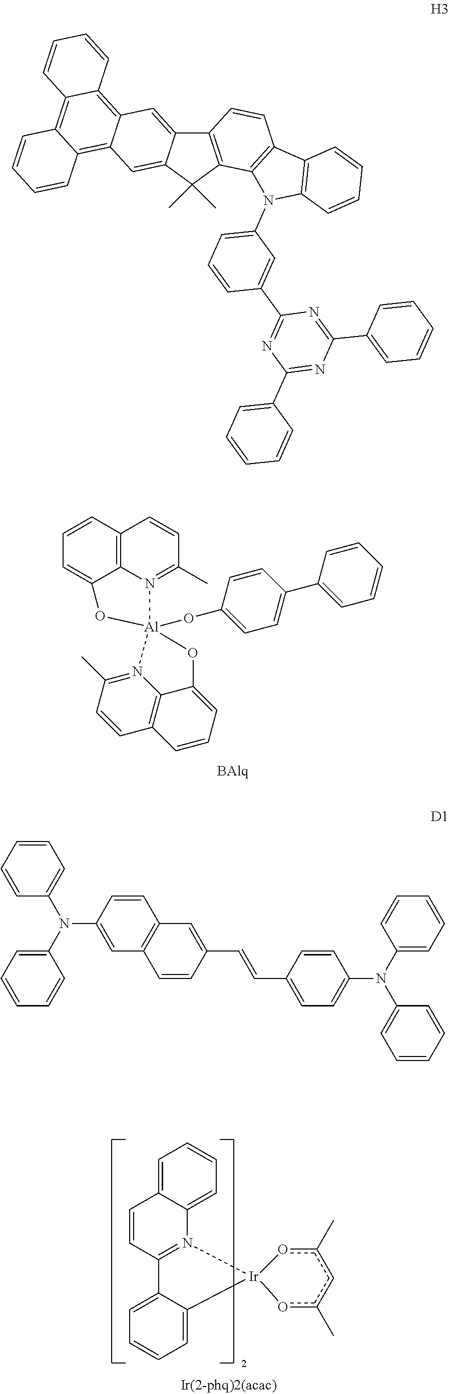

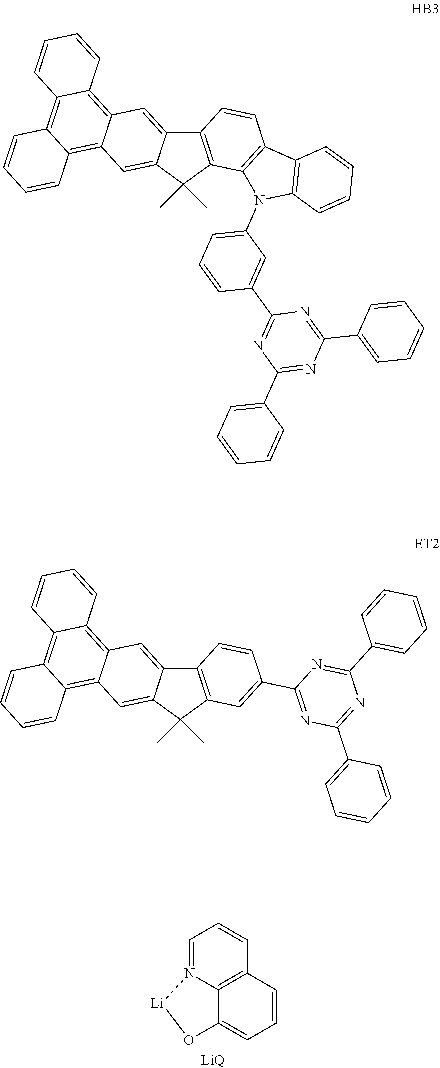

A typical organic EL device consists of low work function metals, such as Al, Mg, Ca, Li and K, as the cathode, and the low work function metals can help electrons injecting the electron transporting layer from cathode. In addition, for reducing the electron injection barrier and improving the organic EL device performance, a thin-film electron injecting layer is introduced between the cathode and the electron transporting layer. The materials of electron injecting layer are metal halide or metal oxide with low work function, such as: LiF, LiQ, MgO, or Li₂O. On the other hand, after the organic EL device fabrication, EL spectra and CIE coordination are measured by using a PR650 spectra scan spectrometer. Furthermore, the current/voltage, luminescence/voltage and yield/voltage characteristics are taken with a Keithley 2400 programmable voltage-current source. The above-mentioned apparatuses are operated at room temperature (about 25° C.) and under atmospheric pressure.

### Example 3

Using a procedure analogous to the above-mentioned general method, organic EL devices emitting fluorescence and having the device structure as shown in the FIGURE. From the bottom layer 10 to the top layer 80, the following components were produced: ITO/HAT-CN (20 nm)/NPB (110 nm)/EB2 (5 nm)/H2 doped with 15% D1 (30 nm)/HB3 (10 nm)/Electron transport material doped with 40% LiQ (35 nm)/LiQ (1 nm)/Al (160 nm).

The hole injection layer 20 (HAT-CN) is deposited onto the transparent electrode 10 (ITO), the hole transport layer 30 (NPB) is deposited onto the hole injection layer 20, the electron blocking layer 40 (EB2) is deposited onto the hole transport layer 30, the emitting layer 50 (H2) is deposited onto the electron blocking layer 40, the hole blocking layer 60 (HB3) is deposited onto the emitting layer 50, the electron transport layer 70 is deposited onto the hole blocking layer 60. The electron transport layer 70 may comprise electron transport material, as shown in, for example, Table 1. The electron transport material may be doped with 40% LiQ. The electron transport layer 70 may have a thickness of about 35 nm. The electron injection layer 80 (LiQ) is deposited onto the electron transport layer 70. The electron injection layer 90 (Al) is deposited onto the electron injection layer 80. The I-V-B (at 1000 nits) and half-life time of fluorescent blue-emitting organic EL device testing report as Table 1, The half-life time is defined that the initial luminance of 1000 cd/m² has dropped to half.

TABLE 1

| Electron | Driving | Current | | |
|---|---|---|---|---|
| Transport | Voltage | Efficiency | Device | Half-life time |
| Material | (V) | (cd/A) | Color | (hours) |
| ET2 | 6 | 5.1 | Blue | 310 |
| A4 | 5.9 | 5.2 | Blue | 340 |
| A7 | 5.5 | 5.4 | Blue | 320 |
| A27 | 4.7 | 5.8 | Blue | 520 |
| A72 | 5.7 | 5.4 | Blue | 360 |
| A91 | 5.4 | 5.3 | Blue | 450 |
| A79 | 5.3 | 5.6 | Blue | 420 |
| A188 | 4.3 | 6.3 | Blue | 580 |
| A162 | 4.5 | 5.5 | Blue | 490 |
| A83 | 4.7 | 6.0 | Blue | 540 |
| A60 | 5.4 | 5.3 | Blue | 500 |
| A35 | 5.3 | 5.7 | Blue | 530 |

Using a procedure analogous to the above-mentioned general method, phosphorescent blue-emitting organic EL device having the device structure as show in the FIGURE. From the ITO/HAT-CN (20 nm)/NPB (110 nm)/EB2 (5 nm)/H2 and H3 doped with 15% Ir(2-phq)₂(acac)(30 nm)/HB3 (10 nm)/ET2 doped with 40% LiQ (35 nm)/LiQ (1 nm)/Al (160 nm). The I-V-B (at 1000 nits) and half-life time test reports of these organic EL devices are summarized in Table 2 below. The half-life time is defined as the time the initial luminance of 1000 cd/m² has dropped to half.

TABLE 2

| Emitting | Hole | Driving | Current | | Half-life |
|---|---|---|---|---|---|
| Host | Blocking | Voltage | Efficiency | Device | time |
| Material | Material | (V) | (cd/A) | Color | (hours) |
| H2 + H3 | HB3 | 4.7 | 19 | Red | 450 |
| A7 + A4 | HB3 | 4.5 | 20 | Red | 470 |
| A7 + A11 | HB3 | 4.4 | 21 | Red | 500 |
| A7 + A139 | HB3 | 4.3 | 22 | Red | 540 |
| A7 + A13 | HB3 | 4.4 | 21 | Red | 460 |
| A7 + A160 | HB3 | 4.5 | 21 | Red | 480 |
| A7 + A12 | HB3 | 4.6 | 20 | Red | 460 |
| A7 + A80 | HB3 | 4.4 | 20 | Red | 520 |
| A7 + A168 | HB3 | 4.5 | 19 | Red | 520 |
| A7 + A129 | HB3 | 4.6 | 21 | Red | 470 |
| A7 + A139 | A4 | 4.3 | 24 | Red | 550 |
| A7 + A139 | A7 | 4.4 | 23 | Red | 520 |
| A7 + A139 | A27 | 4.5 | 23 | Red | 550 |
| A7 + A139 | A79 | 4.6 | 22 | Red | 500 |
| A167 + A139 | A139 | 4.6 | 19 | Red | 490 |
| A110 + A139 | A139 | 4.4 | 21 | Red | 540 |
| A143 + A139 | A139 | 4.2 | 25 | Red | 630 |
| A182 + A139 | A139 | 4.3 | 20 | Red | 530 |
| A171 + A139 | A139 | 4.6 | 22 | Red | 500 |
| A35 + A139 | A139 | 4.5 | 24 | Red | 490 |
| A143 + A139 | — | 4.3 | 23 | Red | 590 |

In Table 1 and Table 2, organic compounds of formula (A) as an electron transport material (ETM), a hole blocking material (HBM), or a phosphorescent blue host material may exhibit better performance than the prior art materials. In particular, an organic EL devices of the present invention comprises an organic compound of formula (A) as an electron transport material, a hole blocking material (HBM), or a phosphorescent blue host material to collocate with an electron transport material ET2, a hole blocking material HB3, or phosphorescent blue host H2 and H3, thereby lowering a driving voltage, increasing a current efficiency or a half-life time under the same voltage of the organic EL device.

Obviously, many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. An organic compound represented by the following formula (A): 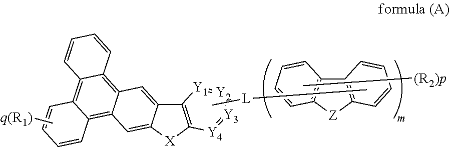
wherein X is a divalent bridge selected from the group consisting of O and S; m represents 0, 1 or 2; L represents a single bond, a substituted or unsubstituted aryl group having 6 to 40 ring carbon atoms, or a substituted or unsubstituted heteroaryl group having 3 to 40 ring carbon atoms if m represents 1 or 2; L represents a substituted or unsubstituted heteroaryl group having 3 to 40 ring carbon atoms if m represents 0; Z represents O, S or NR₃; Y₁ to Y₄ independently represent a nitrogen atom or CR₄. R₃ and R₄ independently represent a hydrogen atom, a substituent, or a bond to L; p represents an integer of 0, 1, 2, 3, 4, 5, 6 or 7; q represents an integer of 0, 1, 2, 3, 4, 5, 6, 7, 8, 9 or 10; and R₁ and R₂ are independently selected from the group consisting of a hydrogen atom, a halide, an alkyl group having 1 to 20 carbon atoms, a substituted or unsubstituted aryl group having 6 to 30 carbon atoms, a substituted or unsubstituted aralkyl group having 7 to 30 carbon atoms, and a substituted or unsubstituted heteroaryl group having 3 to 30 carbon atoms.

2. The organic compound according to claim 1, wherein L is selected from the group consisting of: 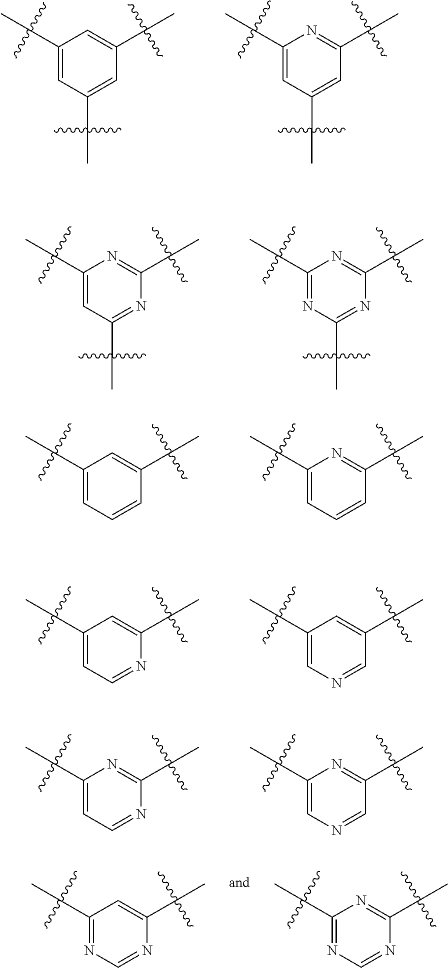
if m represents 1 or 2.

3. The organic compound according to claim 1, wherein L is selected from the group consisting of: 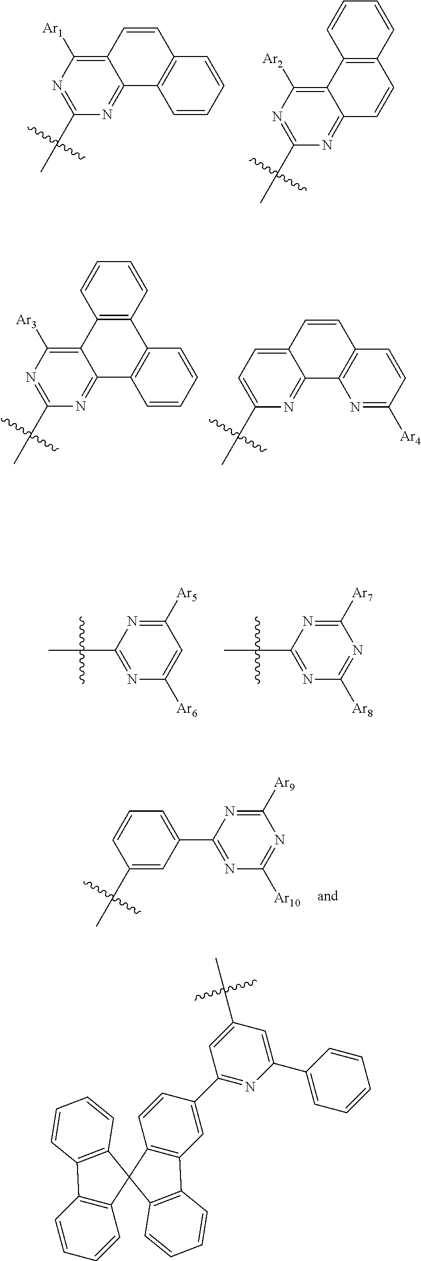
if m represents 0, and wherein Ar₁ to Ar₄ independently represent a hydrogen atom, a substituted or unsubstituted aryl group having 6 to 30 ring carbon atoms, and wherein Ar₅ to Ar₁₀ independently represent a hydrogen atom, a substituted or unsubstituted aryl group having 6 to 30 ring carbon atoms, or a substituted or unsubstituted heteroaryl group having 3 to 30 ring carbon atoms.

4. The organic compound according to claim 3, wherein L is selected from the group consisting of:

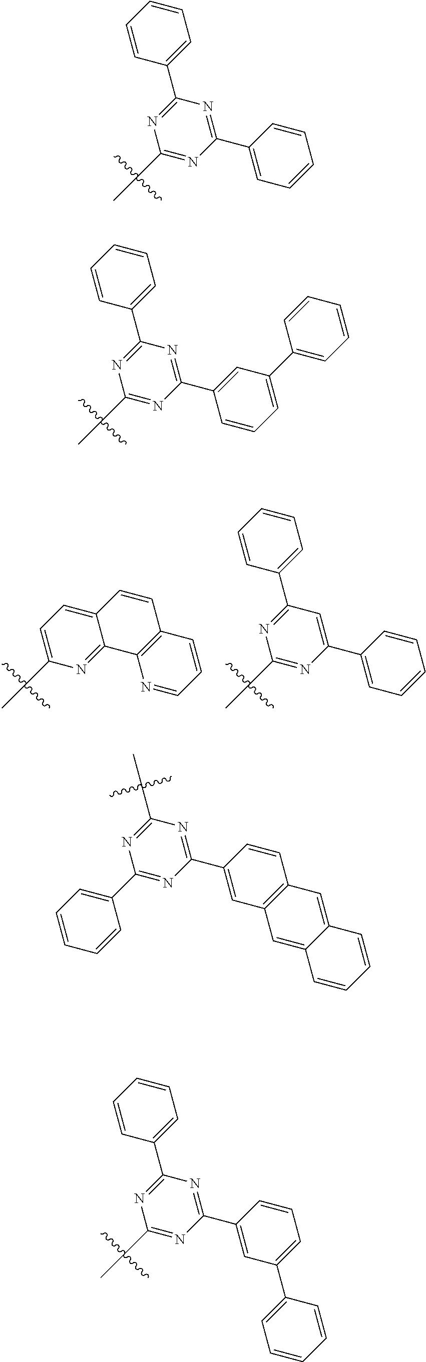 [SMILES: *c1nc(-c2ccccc2)nc(-c2ccccc2)n1 ; *c1nc(-c2ccccc2)nc(-c2cccc(-c3ccccc3)c2)n1 ; *c1ccc2ccc3cccnc3c2n1 ; *c1nc(-c2ccccc2)cc(-c2ccccc2)n1 ; *c1nc(-c2ccccc2)nc(-c2ccc3cc4ccccc4cc3c2)n1 ; *c1nc(-c2ccccc2)nc(-c2cccc(-c3ccccc3)c2)n1]

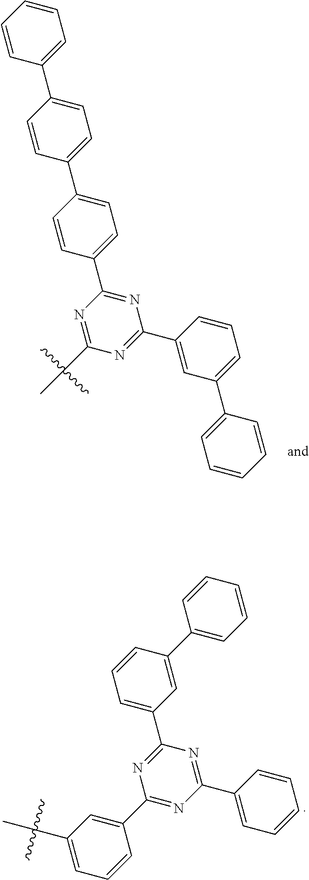 [SMILES: *c1nc(-c2ccc(-c3ccc(-c4ccccc4)cc3)cc2)nc(-c2cccc(-c3ccccc3)c2)n1 ; *c1cccc(-c2nc(-c3ccccc3)nc(-c3cccc(-c4ccccc4)c3)n2)c1]

5. The organic compound according to claim 1, wherein the organic compound is selected from the group consisting of:

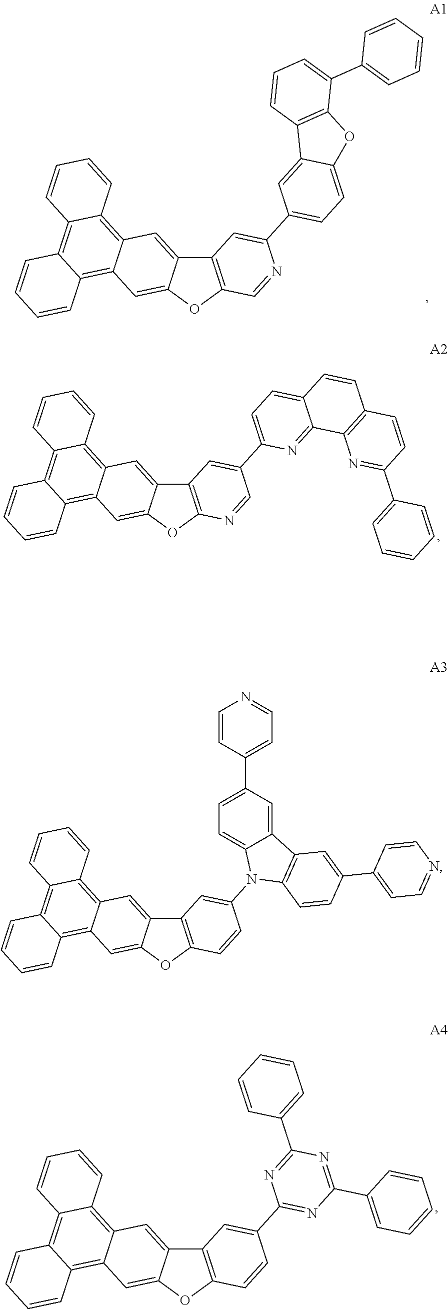 [SMILES: c1ccc(-c2cccc3c2oc2ccc(-c4cc5c(cn4)oc4cc6c7ccccc7c7ccccc7c6cc45)cc23)cc1 ; c1ccc(-c2ccc3ccc4ccc(-c5cnc6oc7cc8c9ccccc9c9ccccc9c8cc7c6c5)nc4c3n2)cc1 ; c1ccc2c(c1)c1ccccc1c1cc3c(cc21)oc1ccc(-n2c4ccc(-c5ccncc5)cc4c4cc(-c5ccncc5)ccc42)cc13 ; c1ccc(-c2nc(-c3ccccc3)nc(-c3ccc4oc5cc6c7ccccc7c7ccccc7c6cc5c4c3)n2)cc1]

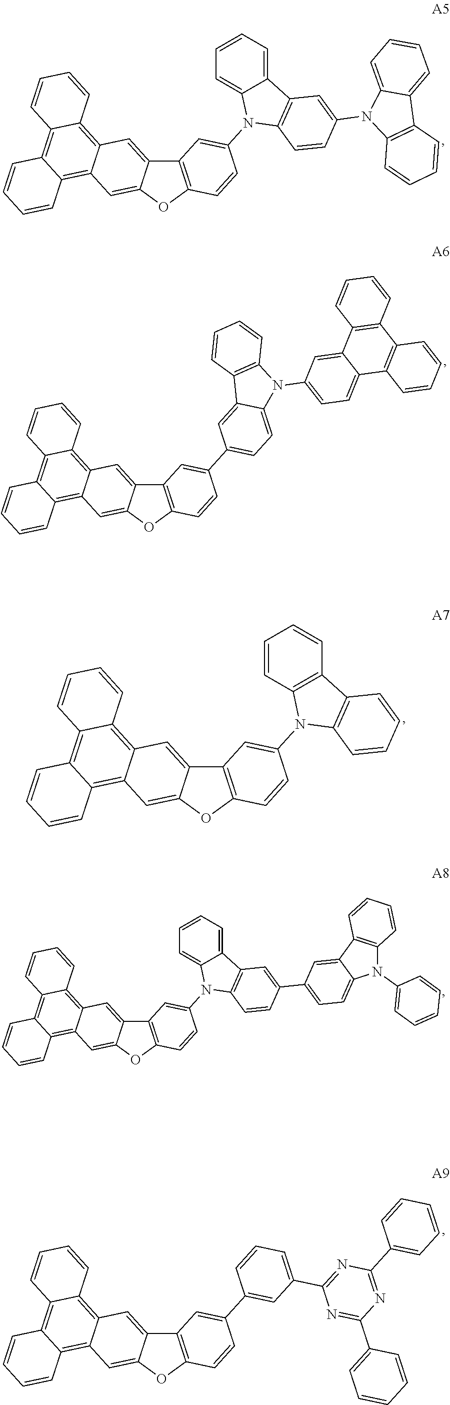 [SMILES: c1ccc2c(c1)c1ccccc1c1cc3c(cc21)oc1ccc(-n2c4ccccc4c4cc(-n5c6ccccc6c6ccccc65)ccc42)cc13 ; c1ccc2c(c1)c1ccccc1c1cc(-n3c4ccccc4c4cc(-c5ccc6oc7cc8c9ccccc9c9ccccc9c8cc7c6c5)ccc43)ccc21 ; c1ccc2c(c1)c1ccccc1c1cc3c(cc21)oc1ccc(-n2c4ccccc4c4ccccc42)cc13 ; c1ccc(-n2c3ccccc3c3cc(-c4ccc5c(c4)c4ccccc4n5-c4ccc5oc6cc7c8ccccc8c8ccccc8c7cc6c5c4)ccc32)cc1 ; c1ccc(-c2nc(-c3ccccc3)nc(-c3cccc(-c4ccc5oc6cc7c8ccccc8c8ccccc8c7cc6c5c4)c3)n2)cc1]

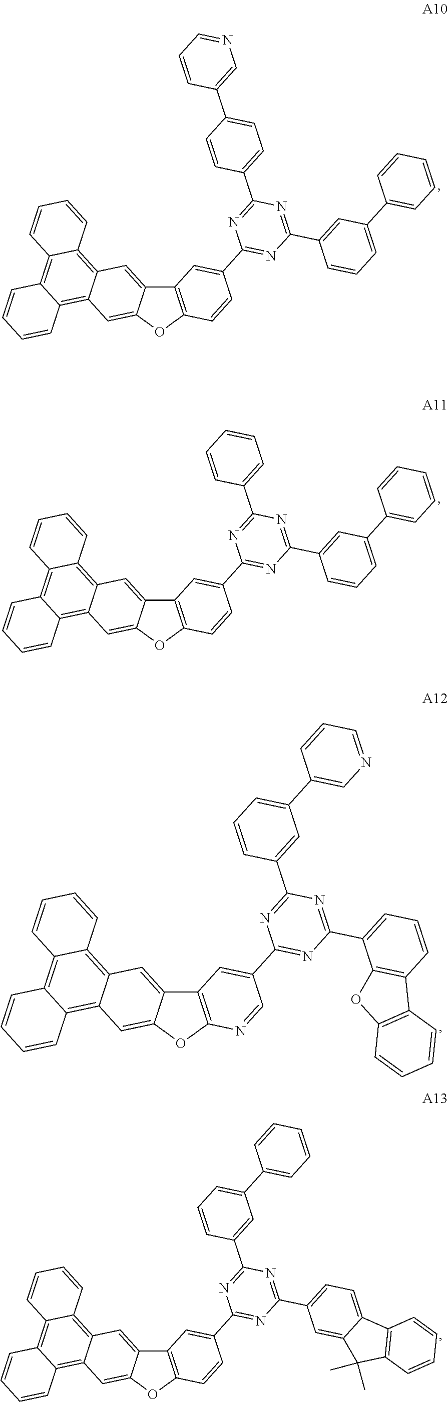 [SMILES: c1ccc(-c2cccc(-c3nc(-c4ccc(-c5cccnc5)cc4)nc(-c4ccc5oc6cc7c8ccccc8c8ccccc8c7cc6c5c4)n3)c2)cc1 ; c1ccc(-c2cccc(-c3nc(-c4ccccc4)nc(-c4ccc5oc6cc7c8ccccc8c8ccccc8c7cc6c5c4)n3)c2)cc1 ; c1cncc(-c2cccc(-c3nc(-c4cnc5oc6cc7c8ccccc8c8ccccc8c7cc6c5c4)nc(-c4cccc5c4oc4ccccc45)n3)c2)c1 ; CC1(C)c2ccccc2-c2ccc(-c3nc(-c4cccc(-c5ccccc5)c4)nc(-c4ccc5oc6cc7c8ccccc8c8ccccc8c7cc6c5c4)n3)cc21]

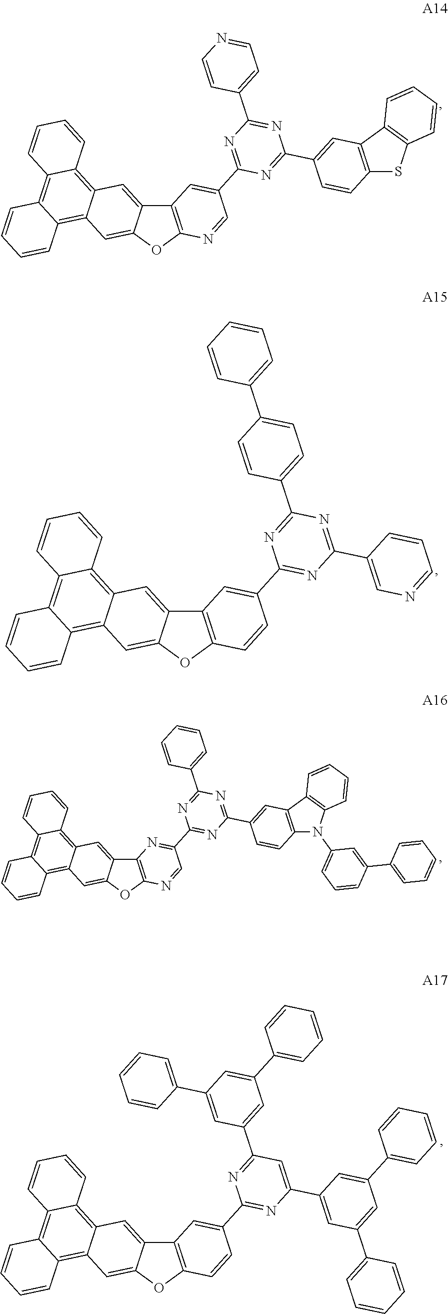 [SMILES: c1ccc2c(c1)sc1ccc(-c3nc(-c4ccncc4)nc(-c4cnc5oc6cc7c8ccccc8c8ccccc8c7cc6c5c4)n3)cc12 ; c1ccc(-c2ccc(-c3nc(-c4cccnc4)nc(-c4ccc5oc6cc7c8ccccc8c8ccccc8c7cc6c5c4)n3)cc2)cc1 ; c1ccc(-c2cccc(-n3c4ccccc4c4cc(-c5nc(-c6ccccc6)nc(-c6cnc7oc8cc9c%10ccccc%10c%10ccccc%10c9cc8c7n6)n5)ccc43)c2)cc1 ; c1ccc(-c2cc(-c3ccccc3)cc(-c3cc(-c4cc(-c5ccccc5)cc(-c5ccccc5)c4)nc(-c4ccc5oc6cc7c8ccccc8c8ccccc8c7cc6c5c4)n3)c2)cc1]

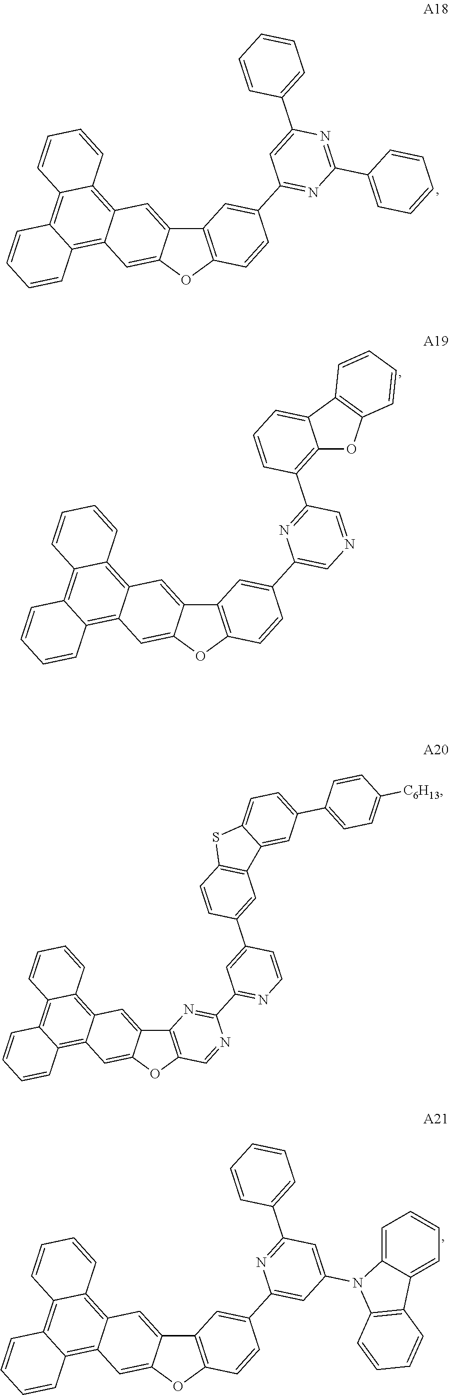

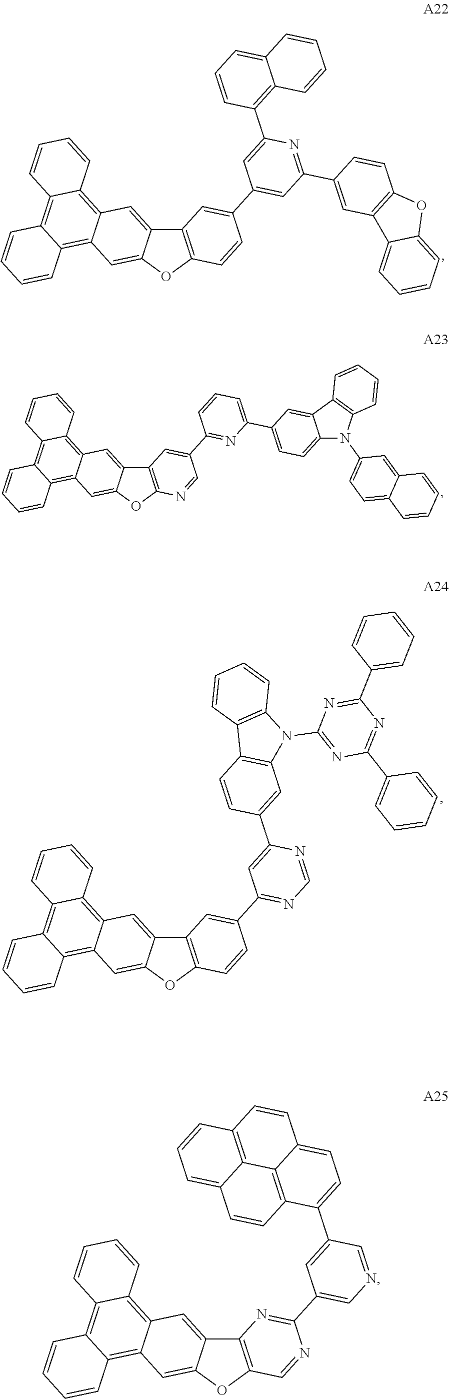 [SMILES: c1ccc2c(-c3cc(-c4ccc5oc6cc7c8ccccc8c8ccccc8c7cc6c5c4)cc(-c4ccc5oc6ccccc6c5c4)n3)cccc2c1 ; c1cc(-c2cnc3oc4cc5c6ccccc6c6ccccc6c5cc4c3c2)nc(-c2ccc3c(c2)c2ccccc2n3-c2ccc3ccccc3c2)c1 ; c1ccc(-c2nc(-c3ccccc3)nc(-n3c4ccccc4c4ccc(-c5cc(-c6ccc7oc8cc9c%10ccccc%10c%10ccccc%10c9cc8c7c6)ncn5)cc43)n2)cc1 ; c1cc2ccc3ccc(-c4cncc(-c5ncc6oc7cc8c9ccccc9c9ccccc9c8cc7c6n5)c4)c4ccc(c1)c2c34]

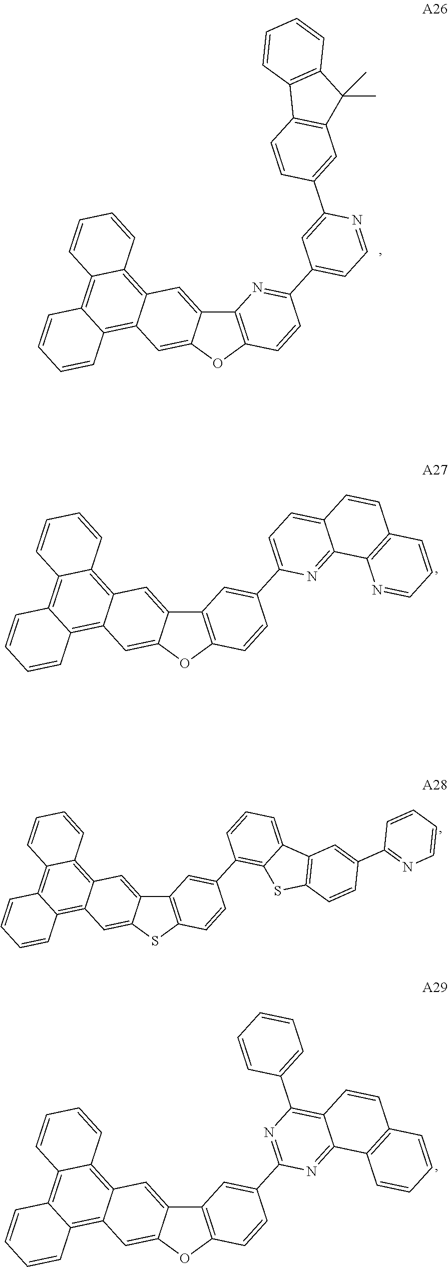 [SMILES: CC1(C)c2ccccc2-c2ccc(-c3cc(-c4ccc5oc6cc7c8ccccc8c8ccccc8c7cc6c5n4)ccn3)cc21 ; c1cnc2c(c1)ccc1ccc(-c3ccc4oc5cc6c7ccccc7c7ccccc7c6cc5c4c3)nc12 ; c1ccc(-c2ccc3sc4c(-c5ccc6sc7cc8c9ccccc9c9ccccc9c8cc7c6c5)cccc4c3c2)nc1 ; c1ccc(-c2nc(-c3ccc4oc5cc6c7ccccc7c7ccccc7c6cc5c4c3)nc3c2ccc2ccccc23)cc1]

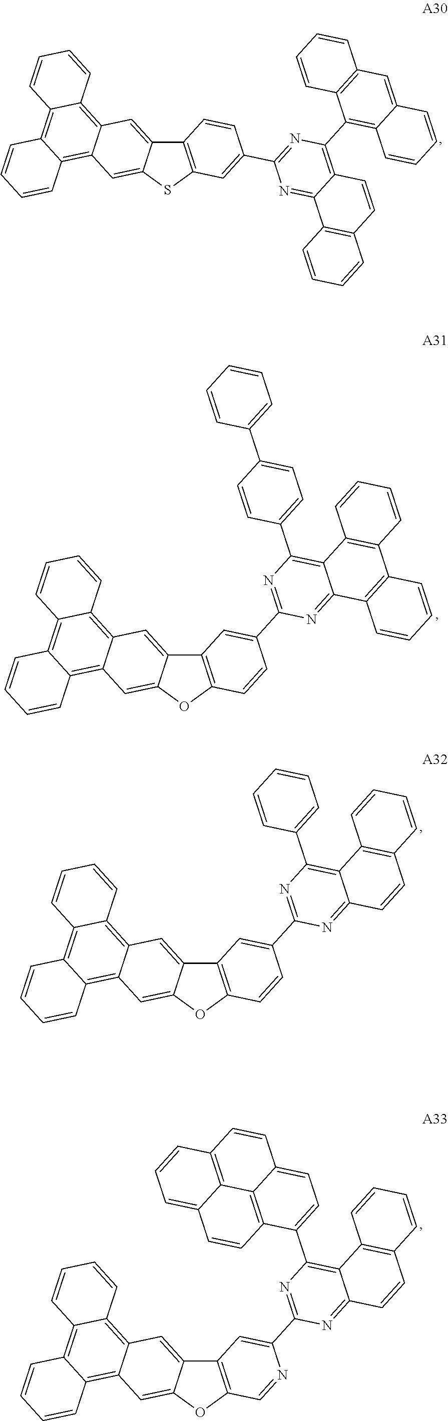 [SMILES: c1ccc2c(-c3nc(-c4ccc5c(c4)sc4cc6c7ccccc7c7ccccc7c6cc45)nc4c3ccc3ccccc34)c3ccccc3cc2c1 ; c1ccc(-c2ccc(-c3nc(-c4ccc5oc6cc7c8ccccc8c8ccccc8c7cc6c5c4)nc4c5ccccc5c5ccccc5c34)cc2)cc1 ; c1ccc(-c2nc(-c3ccc4oc5cc6c7ccccc7c7ccccc7c6cc5c4c3)nc3ccc4ccccc4c23)cc1 ; c1ccc2c(c1)ccc1nc(-c3cc4c(cn3)oc3cc5c6ccccc6c6ccccc6c5cc34)nc(-c3ccc4ccc5cccc6ccc3c4c56)c12]

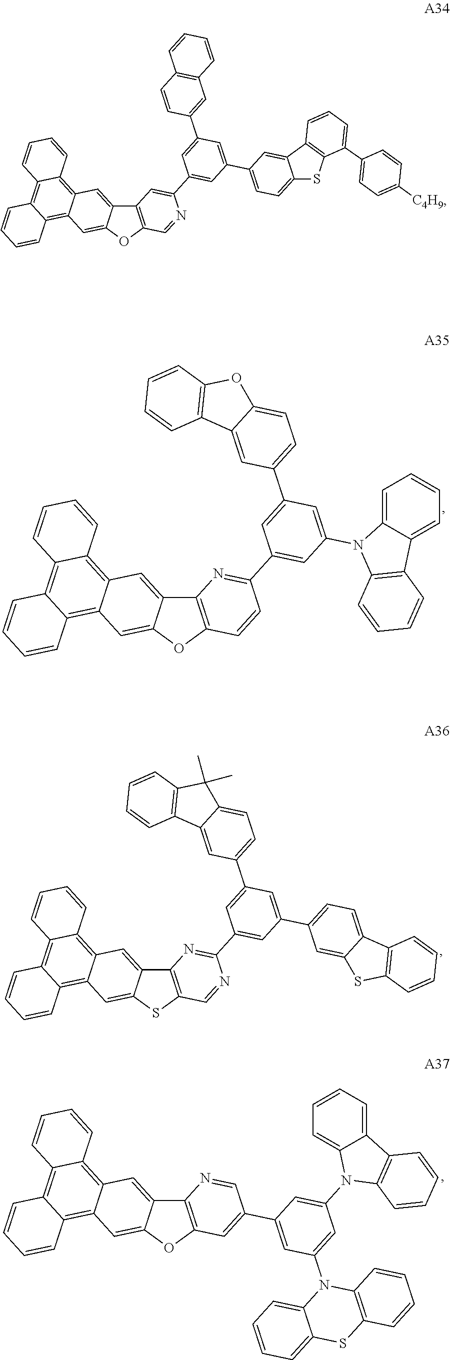

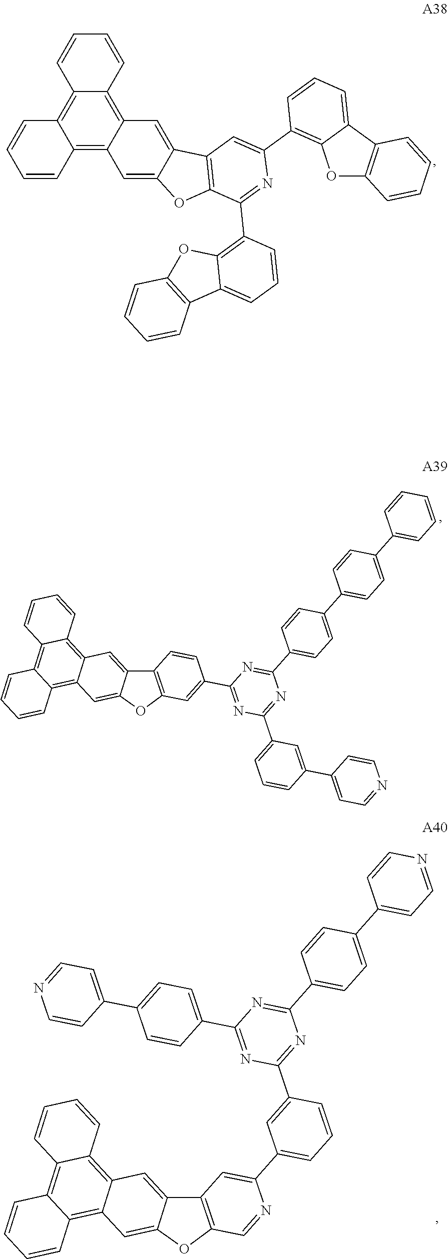 [SMILES: c1ccc2c(c1)oc1c(-c3cc4c(oc5cc6c7ccccc7c7ccccc7c6cc54)c(-c4cccc5c4oc4ccccc45)n3)cccc12 ; c1ccc(-c2ccc(-c3ccc(-c4nc(-c5cccc(-c6ccncc6)c5)nc(-c5ccc6c(c5)oc5cc7c8ccccc8c8ccccc8c7cc56)n4)cc3)cc2)cc1 ; c1cc(-c2cc3c(cn2)oc2cc4c5ccccc5c5ccccc5c4cc23)cc(-c2nc(-c3ccc(-c4ccncc4)cc3)nc(-c3ccc(-c4ccncc4)cc3)n2)c1]

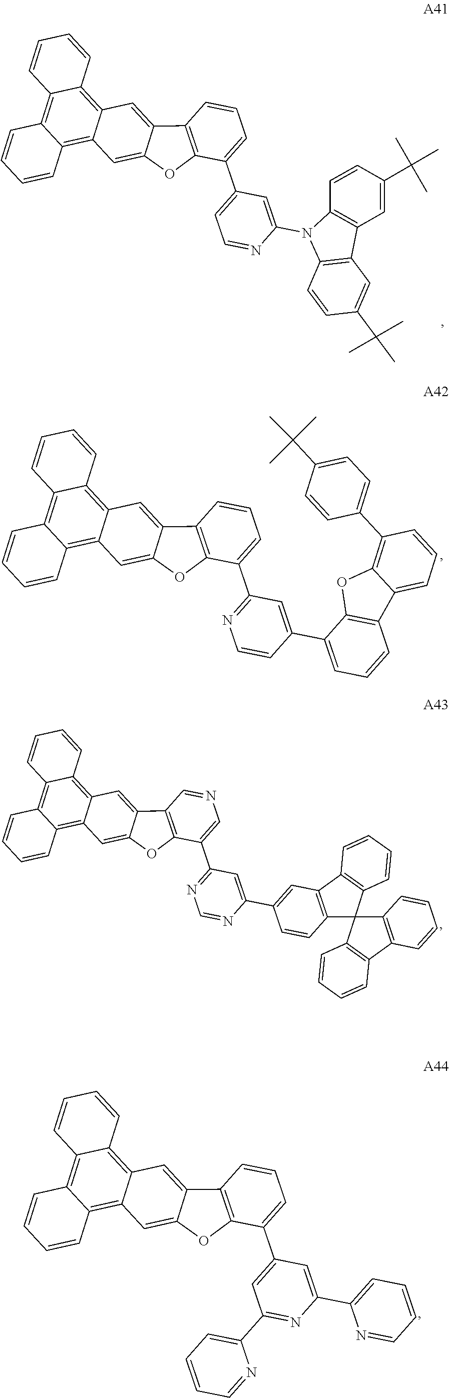 [SMILES: CC(C)(C)c1ccc2c(c1)c1cc(C(C)(C)C)ccc1n2-c1cc(-c2cccc3c2oc2cc4c5ccccc5c5ccccc5c4cc23)ccn1 ; CC(C)(C)c1ccc(-c2cccc3c2oc2c(-c4ccnc(-c5cccc6c5oc5cc7c8ccccc8c8ccccc8c7cc56)c4)cccc23)cc1 ; c1ccc2c(c1)-c1ccccc1C21c2ccccc2-c2cc(-c3cc(-c4cncc5c4oc4cc6c7ccccc7c7ccccc7c6cc45)ncn3)ccc21 ; c1ccc(-c2cc(-c3cccc4c3oc3cc5c6ccccc6c6ccccc6c5cc34)cc(-c3ccccn3)n2)nc1]

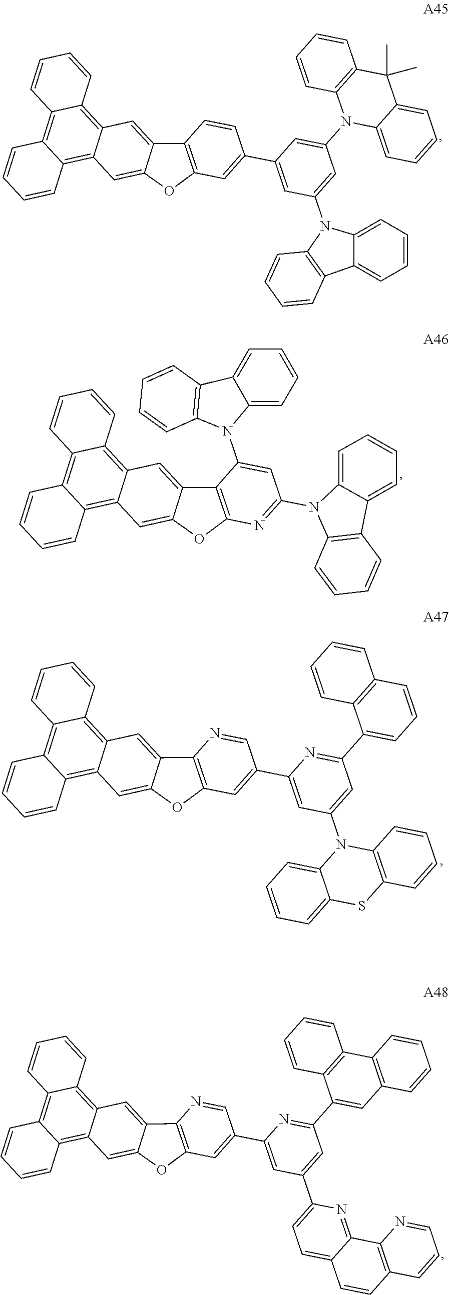

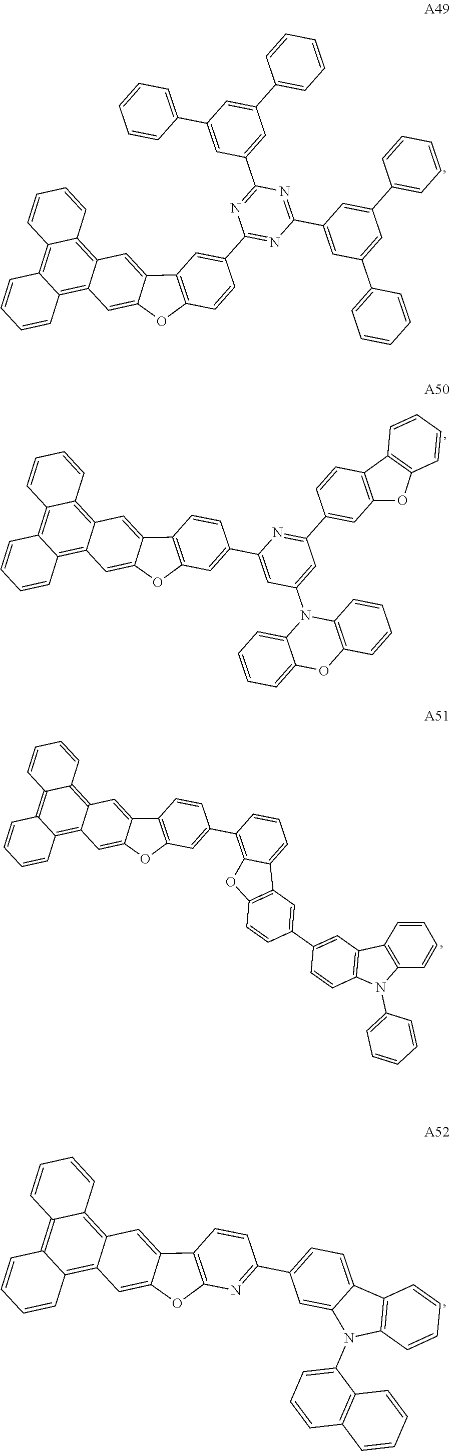 [SMILES: c1ccc(-c2cc(-c3ccccc3)cc(-c3nc(-c4cc(-c5ccccc5)cc(-c5ccccc5)c4)nc(-c4ccc5oc6cc7c8ccccc8c8ccccc8c7cc6c5c4)n3)c2)cc1 ; c1ccc2c(c1)Oc1ccccc1N2c1cc(-c2ccc3c(c2)oc2ccccc23)nc(-c2ccc3c(c2)oc2cc4c5ccccc5c5ccccc5c4cc23)c1 ; c1ccc(-n2c3ccccc3c3cc(-c4ccc5oc6c(-c7ccc8c(c7)oc7cc9c%10ccccc%10c%10ccccc%10c9cc78)cccc6c5c4)ccc32)cc1 ; c1ccc2c(-n3c4ccccc4c4ccc(-c5ccc6c(n5)oc5cc7c8ccccc8c8ccccc8c7cc56)cc43)cccc2c1]

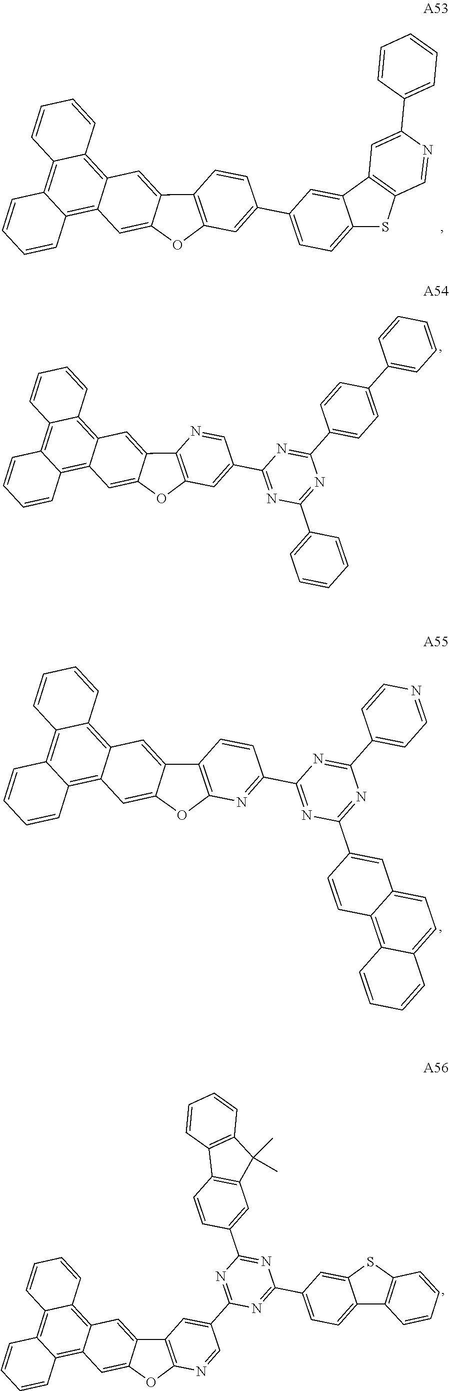 [SMILES: c1ccc(-c2cc3c(cn2)sc2ccc(-c4ccc5c(c4)oc4cc6c7ccccc7c7ccccc7c6cc45)cc23)cc1 ; c1ccc(-c2ccc(-c3nc(-c4ccccc4)nc(-c4cnc5c(c4)oc4cc6c7ccccc7c7ccccc7c6cc45)n3)cc2)cc1 ; c1ccc2c(c1)ccc1cc(-c3nc(-c4ccncc4)nc(-c4ccc5c(n4)oc4cc6c7ccccc7c7ccccc7c6cc45)n3)ccc12 ; CC1(C)c2ccccc2-c2ccc(-c3nc(-c4ccc5c(c4)sc4ccccc45)nc(-c4cnc5oc6cc7c8ccccc8c8ccccc8c7cc6c5c4)n3)cc21]

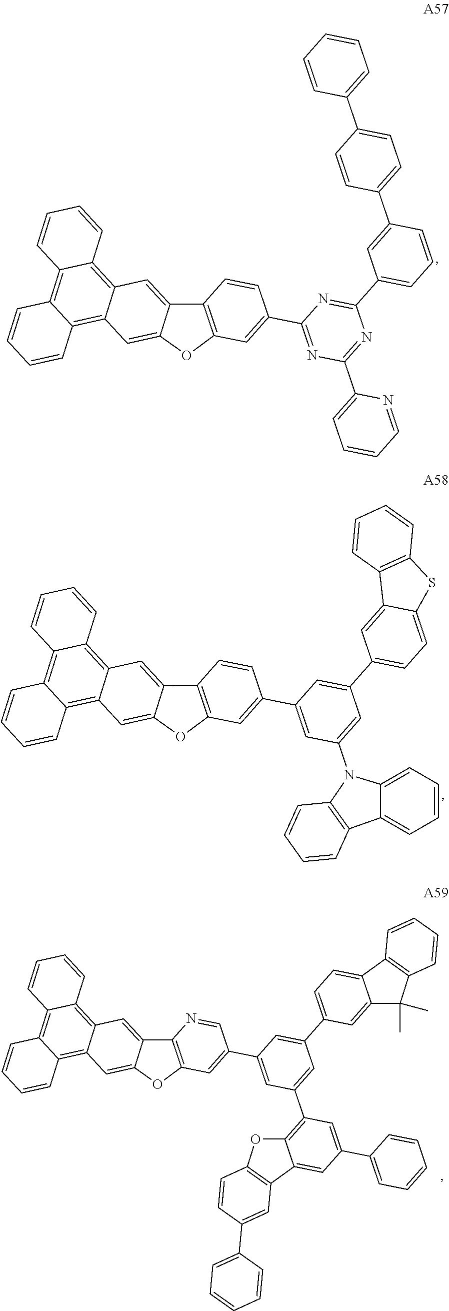 [SMILES: c1ccc(-c2ccc(-c3cccc(-c4nc(-c5ccc6c(c5)oc5cc7c8ccccc8c8ccccc8c7cc56)nc(-c5ccccn5)n4)c3)cc2)cc1 ; c1ccc2c(c1)sc1ccc(-c3cc(-c4ccc5c(c4)oc4cc6c7ccccc7c7ccccc7c6cc45)cc(-n4c5ccccc5c5ccccc54)c3)cc12 ; CC1(C)c2ccccc2-c2ccc(-c3cc(-c4cnc5c(c4)oc4cc6c7ccccc7c7ccccc7c6cc45)cc(-c4cc(-c5ccccc5)cc5c4oc4ccc(-c6ccccc6)cc45)c3)cc21]

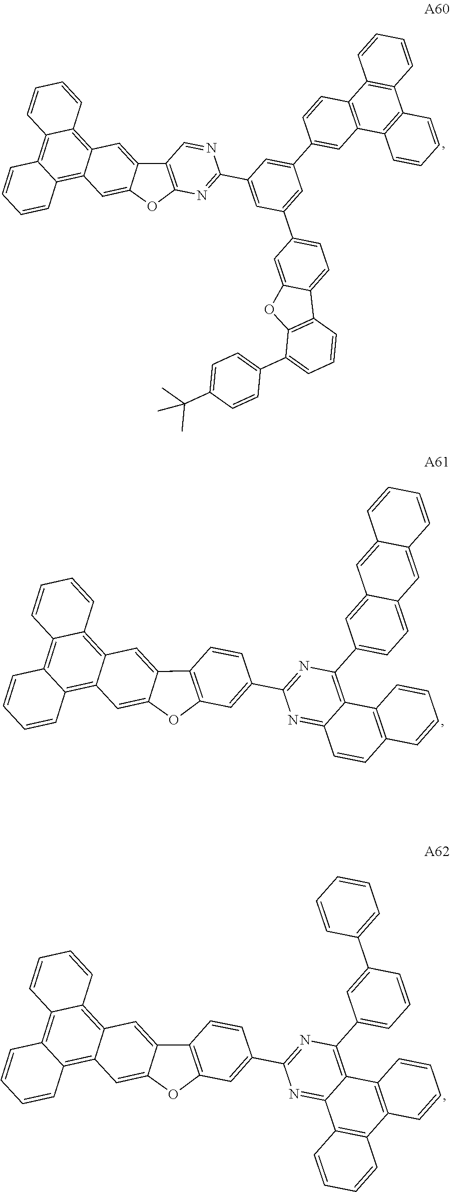 [SMILES: CC(C)(C)c1ccc(-c2cccc3c2oc2cc(-c4cc(-c5ccc6c7ccccc7c7ccccc7c6c5)cc(-c5ncc6c(n5)oc5cc7c8ccccc8c8ccccc8c7cc56)c4)ccc23)cc1 ; c1ccc2cc3cc(-c4nc(-c5ccc6c(c5)oc5cc7c8ccccc8c8ccccc8c7cc56)nc5ccc6ccccc6c45)ccc3cc2c1 ; c1ccc(-c2cccc(-c3nc(-c4ccc5c(c4)oc4cc6c7ccccc7c7ccccc7c6cc45)nc4c5ccccc5c5ccccc5c34)c2)cc1]

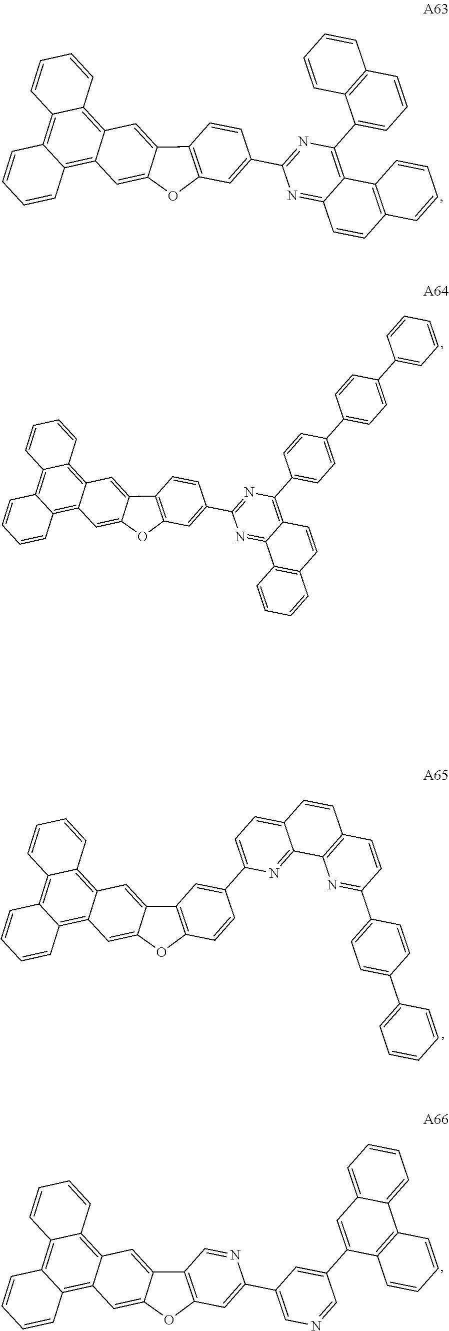 [SMILES: c1ccc2c(-c3nc(-c4ccc5c(c4)oc4cc6c7ccccc7c7ccccc7c6cc45)nc4ccc5ccccc5c34)cccc2c1 ; c1ccc(-c2ccc(-c3ccc(-c4nc(-c5ccc6c(c5)oc5cc7c8ccccc8c8ccccc8c7cc56)nc5c4ccc4ccccc45)cc3)cc2)cc1 ; c1ccc(-c2ccc(-c3ccc4ccc5ccc(-c6ccc7oc8cc9c%10ccccc%10c%10ccccc%10c9cc8c7c6)nc5c4n3)cc2)cc1 ; c1ccc2c(c1)cc(-c1cncc(-c3cc4oc5cc6c7ccccc7c7ccccc7c6cc5c4cn3)c1)c1ccccc12]

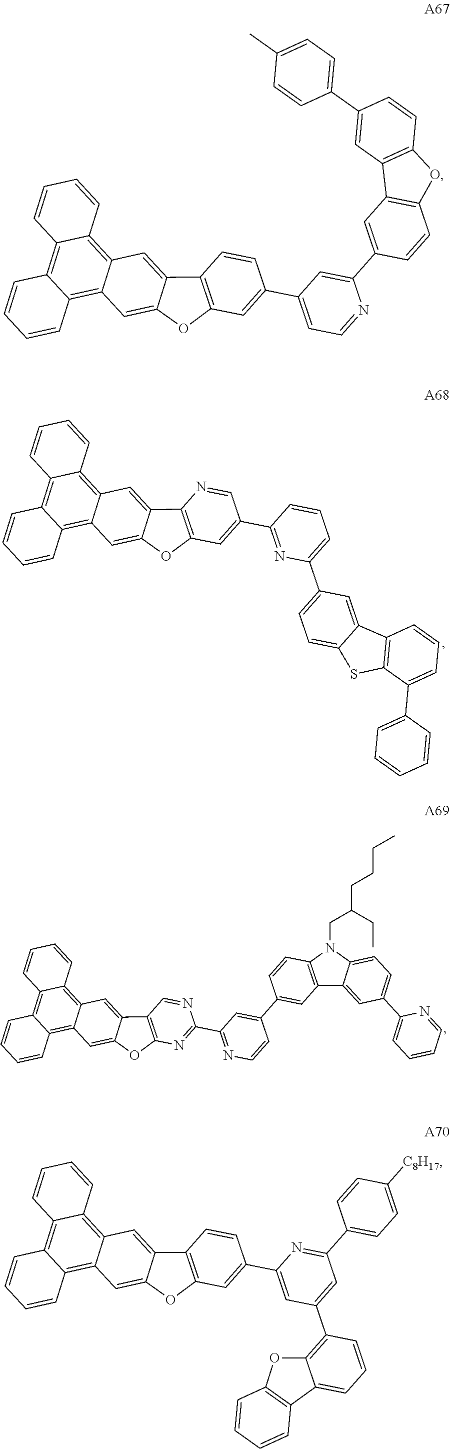

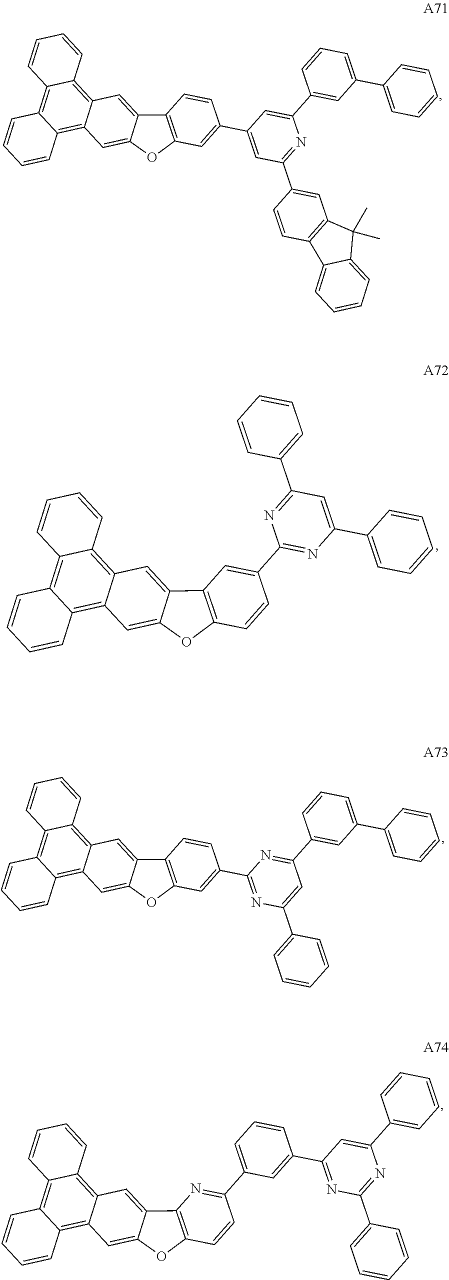 [SMILES: CC1(C)c2ccccc2-c2ccc(-c3cc(-c4ccc5c(c4)oc4cc6c7ccccc7c7ccccc7c6cc45)cc(-c4cccc(-c5ccccc5)c4)n3)cc21 ; c1ccc(-c2cc(-c3ccccc3)nc(-c3ccc4oc5cc6c7ccccc7c7ccccc7c6cc5c4c3)n2)cc1 ; c1ccc(-c2cccc(-c3cc(-c4ccccc4)nc(-c4ccc5c(c4)oc4cc6c7ccccc7c7ccccc7c6cc45)n3)c2)cc1 ; c1ccc(-c2cc(-c3cccc(-c4ccc5oc6cc7c8ccccc8c8ccccc8c7cc6c5n4)c3)nc(-c3ccccc3)n2)cc1]

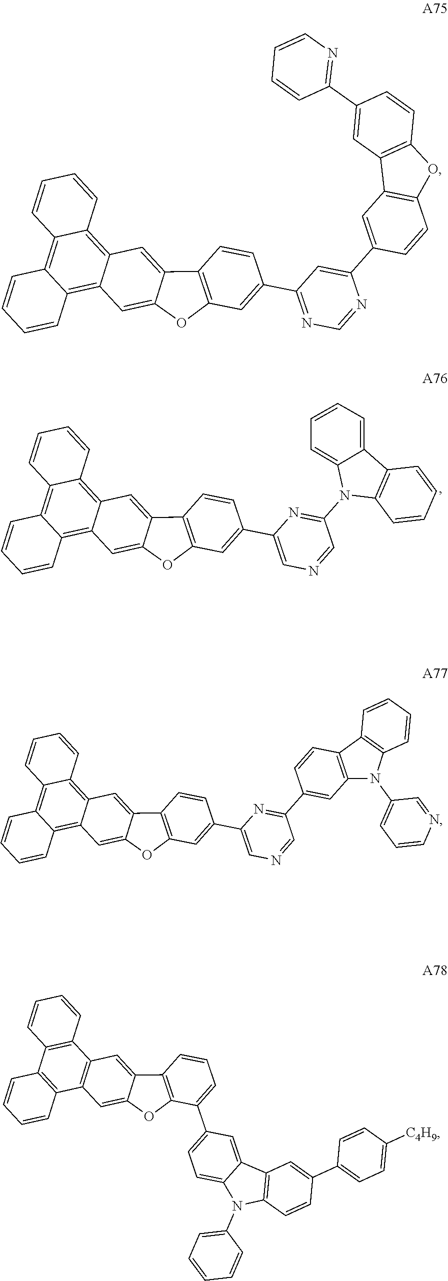

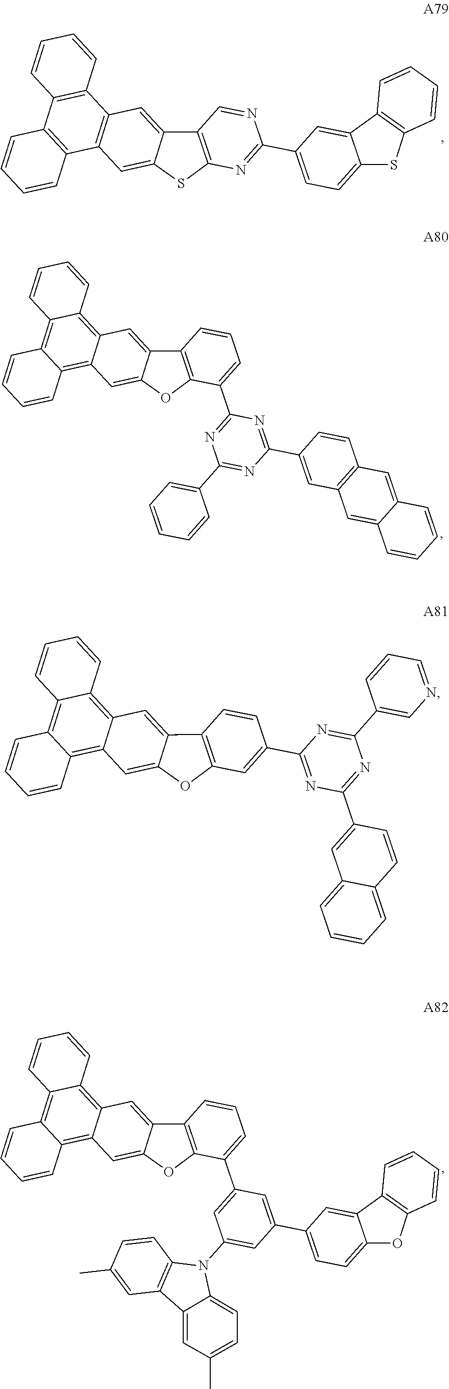 [SMILES: c1ccc2c(c1)sc1ccc(-c3ncc4c(n3)sc3cc5c6ccccc6c6ccccc6c5cc34)cc12 ; c1ccc(-c2nc(-c3ccc4cc5ccccc5cc4c3)nc(-c3cccc4c3oc3cc5c6ccccc6c6ccccc6c5cc34)n2)cc1 ; c1cncc(-c2nc(-c3ccc4ccccc4c3)nc(-c3ccc4c(c3)oc3cc5c6ccccc6c6ccccc6c5cc34)n2)c1 ; Cc1ccc2c(c1)c1cc(C)ccc1n2-c1cc(-c2ccc3oc4ccccc4c3c2)cc(-c2cccc3c2oc2cc4c5ccccc5c5ccccc5c4cc23)c1]

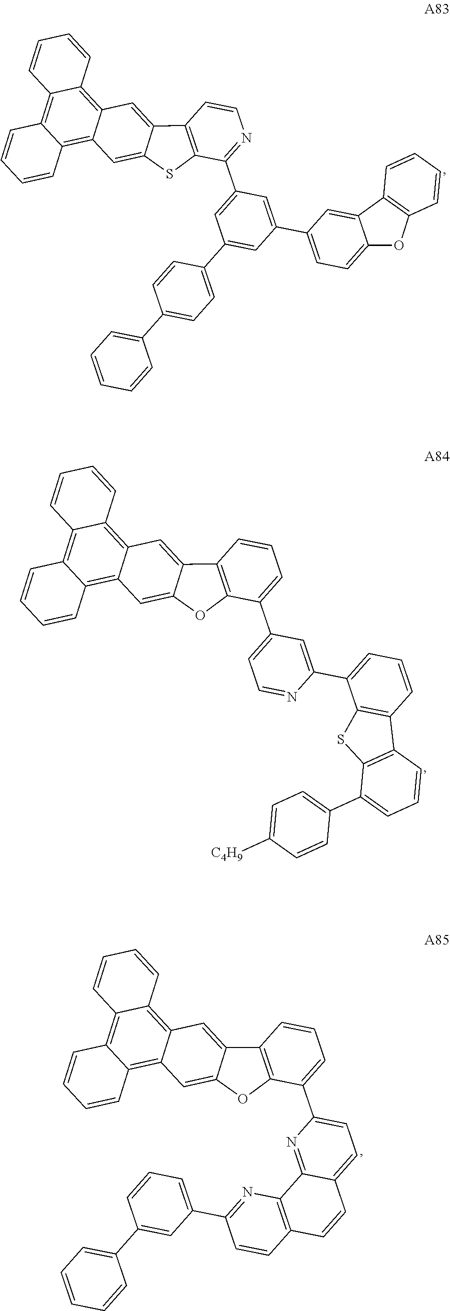

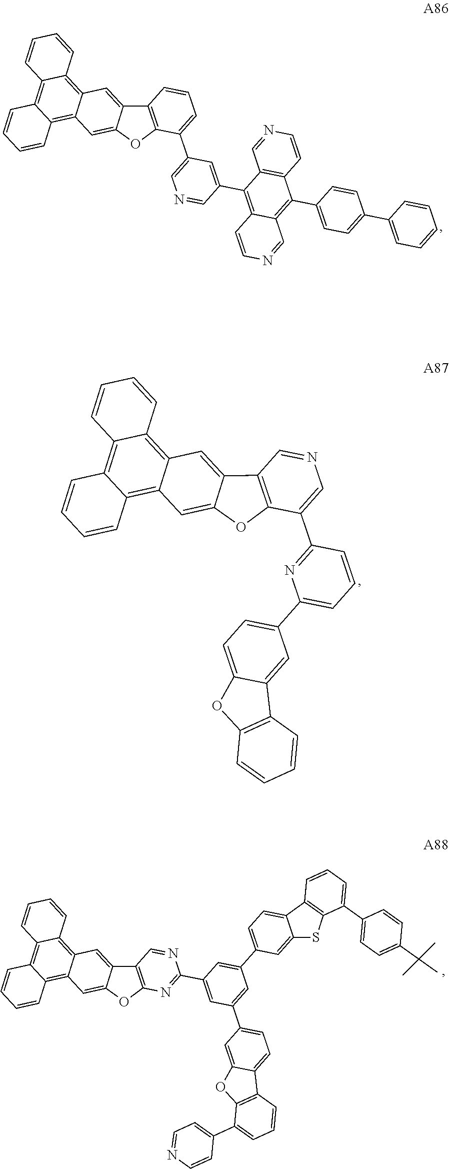 [SMILES: c1ccc(-c2ccc(-c3c4ccncc4c(-c4cncc(-c5cccc6c5oc5cc7c8ccccc8c8ccccc8c7cc56)c4)c4ccncc34)cc2)cc1 ; c1cc(-c2ccc3oc4ccccc4c3c2)nc(-c2cncc3c2oc2cc4c5ccccc5c5ccccc5c4cc23)c1 ; CC(C)(C)c1ccc(-c2cccc3c2sc2cc(-c4cc(-c5ccc6c(c5)oc5c(-c7ccncc7)cccc56)cc(-c5ncc6c(n5)oc5cc7c8ccccc8c8ccccc8c7cc56)c4)ccc23)cc1]

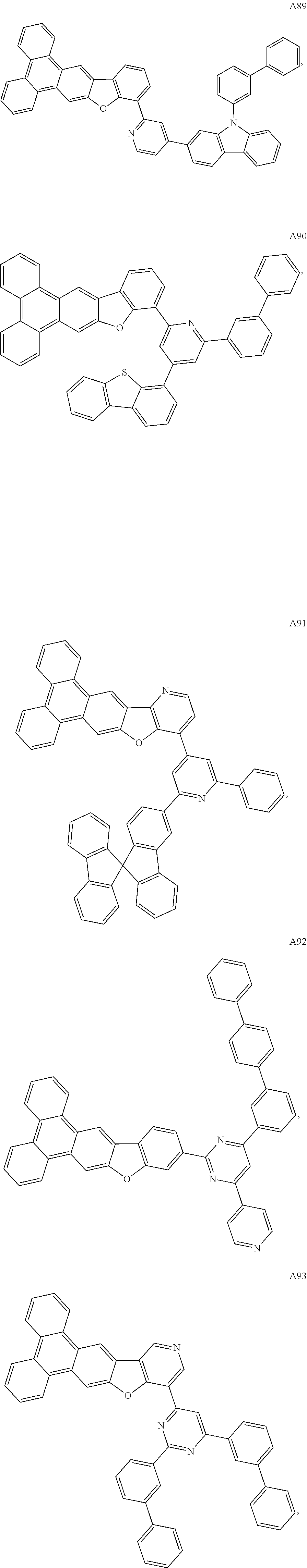 [SMILES: c1ccc(-c2cccc(-n3c4ccccc4c4ccc(-c5ccnc(-c6cccc7c6oc6cc8c9ccccc9c9ccccc9c8cc67)c5)cc43)c2)cc1 ; c1ccc(-c2cccc(-c3cc(-c4cccc5c4sc4ccccc45)cc(-c4cccc5c4oc4cc6c7ccccc7c7ccccc7c6cc45)n3)c2)cc1 ; c1ccc(-c2cc(-c3ccnc4c3oc3cc5c6ccccc6c6ccccc6c5cc34)cc(-c3ccc4c(c3)-c3ccccc3C43c4ccccc4-c4ccccc43)n2)cc1 ; c1ccc(-c2ccc(-c3cccc(-c4cc(-c5ccncc5)nc(-c5ccc6c(c5)oc5cc7c8ccccc8c8ccccc8c7cc56)n4)c3)cc2)cc1 ; c1ccc(-c2cccc(-c3cc(-c4cncc5c4oc4cc6c7ccccc7c7ccccc7c6cc45)nc(-c4cccc(-c5ccccc5)c4)n3)c2)cc1]

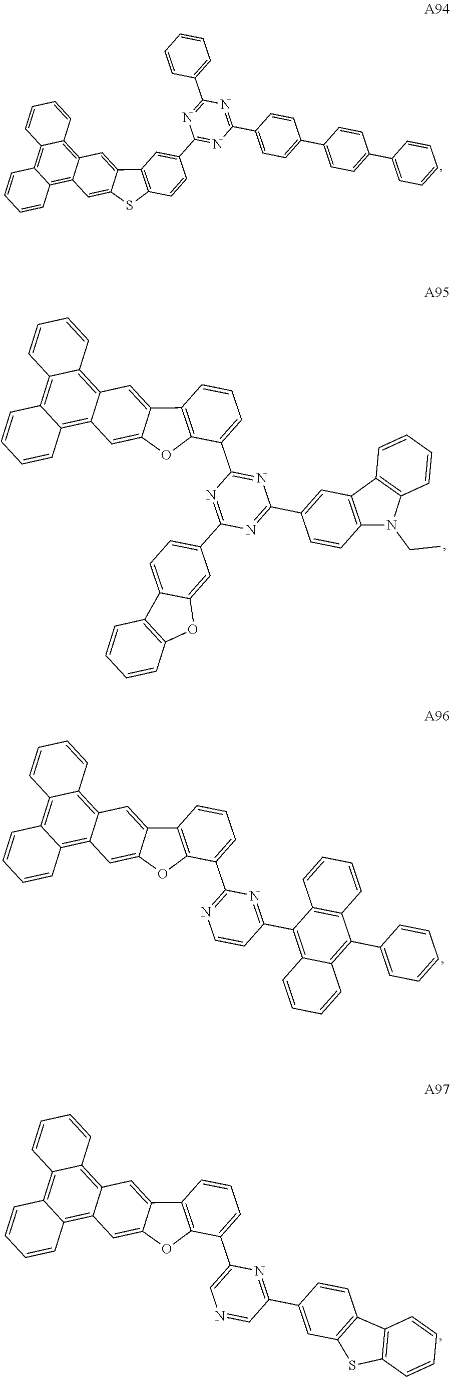 [SMILES: c1ccc(-c2ccc(-c3ccc(-c4nc(-c5ccccc5)nc(-c5ccc6sc7cc8c9ccccc9c9ccccc9c8cc7c6c5)n4)cc3)cc2)cc1 ; CCn1c2ccccc2c2cc(-c3nc(-c4ccc5c(c4)oc4ccccc45)nc(-c4cccc5c4oc4cc6c7ccccc7c7ccccc7c6cc45)n3)ccc21 ; c1ccc(-c2c3ccccc3c(-c3ccnc(-c4cccc5c4oc4cc6c7ccccc7c7ccccc7c6cc45)n3)c3ccccc23)cc1 ; c1ccc2c(c1)sc1cc(-c3cncc(-c4cccc5c4oc4cc6c7ccccc7c7ccccc7c6cc45)n3)ccc12]

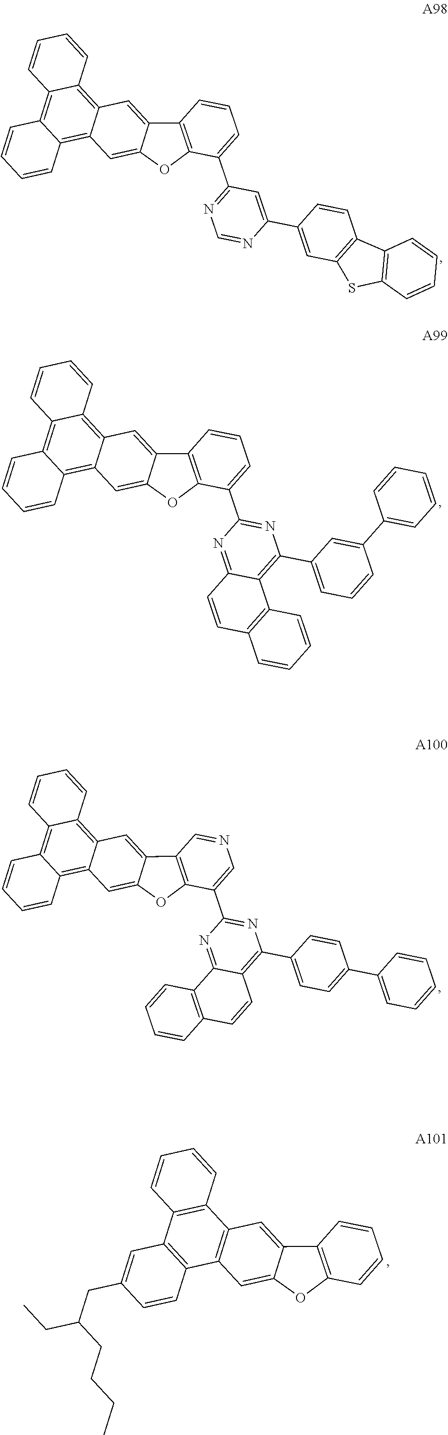 [SMILES: c1ccc2c(c1)sc1cc(-c3cc(-c4cccc5c4oc4cc6c7ccccc7c7ccccc7c6cc45)ncn3)ccc12 ; c1ccc(-c2cccc(-c3nc(-c4cccc5c4oc4cc6c7ccccc7c7ccccc7c6cc45)nc4ccc5ccccc5c34)c2)cc1 ; c1ccc(-c2ccc(-c3nc(-c4cncc5c4oc4cc6c7ccccc7c7ccccc7c6cc45)nc4c3ccc3ccccc34)cc2)cc1 ; CCCCC(CC)Cc1ccc2c(c1)c1ccccc1c1cc3c(cc21)oc1ccccc13]

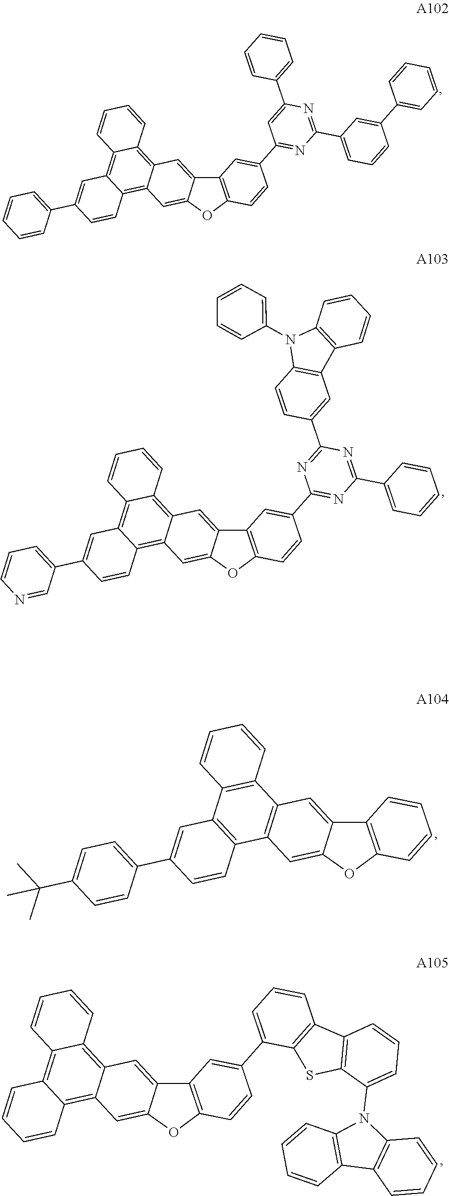 [SMILES: c1ccc(-c2cccc(-c3nc(-c4ccccc4)cc(-c4ccc5oc6cc7c8ccc(-c9ccccc9)cc8c8ccccc8c7cc6c5c4)n3)c2)cc1 ; c1ccc(-c2nc(-c3ccc4oc5cc6c7ccc(-c8cccnc8)cc7c7ccccc7c6cc5c4c3)nc(-c3ccc4c(c3)c3ccccc3n4-c3ccccc3)n2)cc1 ; CC(C)(C)c1ccc(-c2ccc3c(c2)c2ccccc2c2cc4c(cc32)oc2ccccc24)cc1 ; c1cc(-c2ccc3oc4cc5c6ccccc6c6ccccc6c5cc4c3c2)c2sc3c(-n4c5ccccc5c5ccccc54)cccc3c2c1]

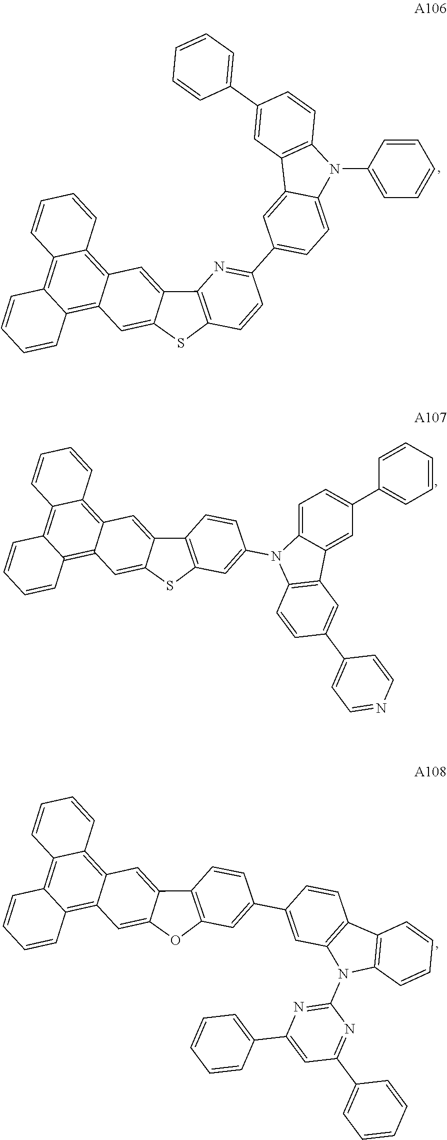 [SMILES: c1ccc(-c2ccc3c(c2)c2cc(-c4ccc5sc6cc7c8ccccc8c8ccccc8c7cc6c5n4)ccc2n3-c2ccccc2)cc1 ; c1ccc(-c2ccc3c(c2)c2cc(-c4ccncc4)ccc2n3-c2ccc3c(c2)sc2cc4c5ccccc5c5ccccc5c4cc23)cc1 ; c1ccc(-c2cc(-c3ccccc3)nc(-n3c4ccccc4c4ccc(-c5ccc6c(c5)oc5cc7c8ccccc8c8ccccc8c7cc56)cc43)n2)cc1]

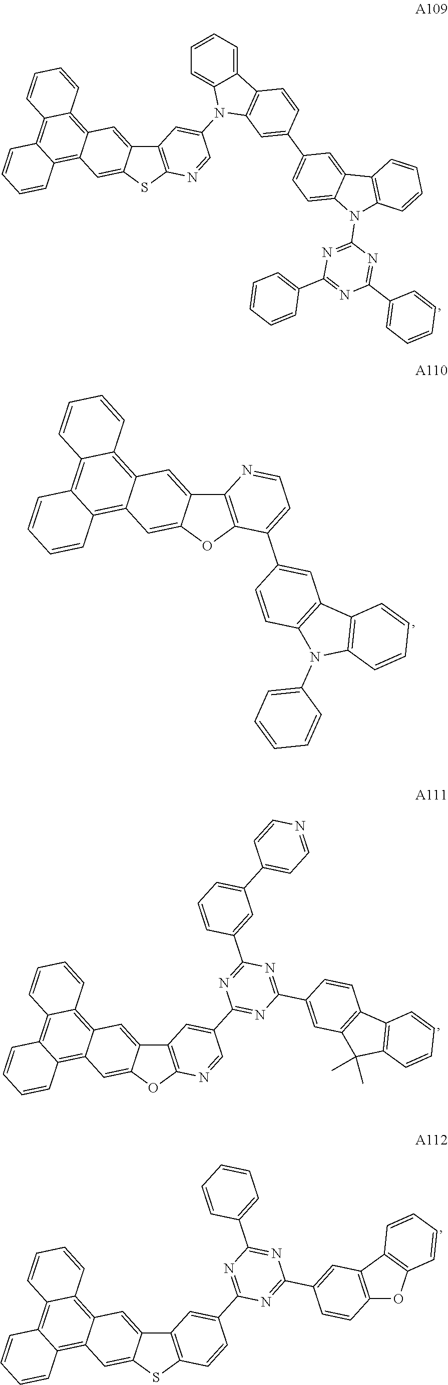 [SMILES: c1ccc(-c2nc(-c3ccccc3)nc(-n3c4ccccc4c4cc(-c5ccc6c7ccccc7n(-c7cnc8sc9cc%10c%11ccccc%11c%11ccccc%11c%10cc9c8c7)c6c5)ccc43)n2)cc1 ; c1ccc(-n2c3ccccc3c3cc(-c4ccnc5c4oc4cc6c7ccccc7c7ccccc7c6cc45)ccc32)cc1 ; CC1(C)c2ccccc2-c2ccc(-c3nc(-c4cccc(-c5ccncc5)c4)nc(-c4cnc5oc6cc7c8ccccc8c8ccccc8c7cc6c5c4)n3)cc21 ; c1ccc(-c2nc(-c3ccc4oc5ccccc5c4c3)nc(-c3ccc4sc5cc6c7ccccc7c7ccccc7c6cc5c4c3)n2)cc1]

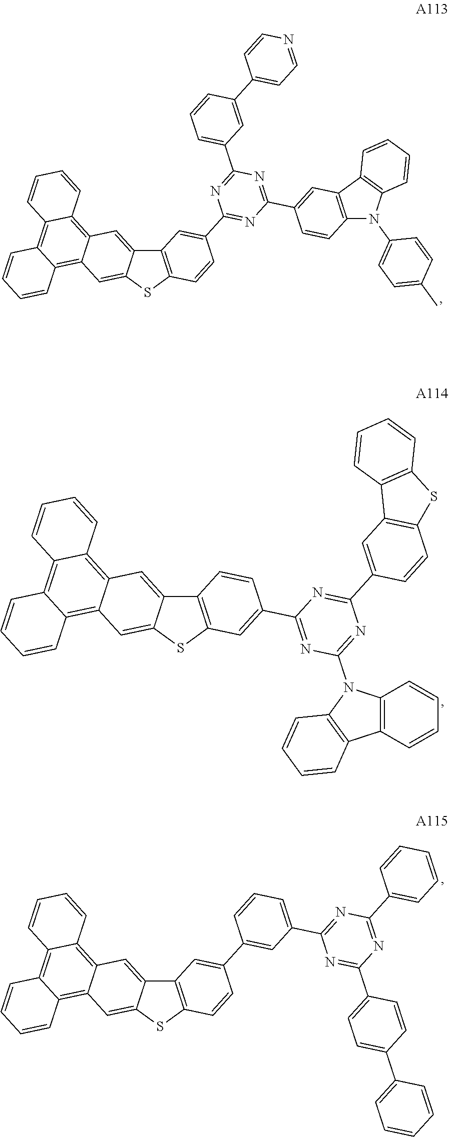 [SMILES: Cc1ccc(-n2c3ccccc3c3cc(-c4nc(-c5cccc(-c6ccncc6)c5)nc(-c5ccc6sc7cc8c9ccccc9c9ccccc9c8cc7c6c5)n4)ccc32)cc1 ; c1ccc2c(c1)sc1ccc(-c3nc(-c4ccc5c(c4)sc4cc6c7ccccc7c7ccccc7c6cc45)nc(-n4c5ccccc5c5ccccc54)n3)cc12 ; c1ccc(-c2ccc(-c3nc(-c4ccccc4)nc(-c4cccc(-c5ccc6sc7cc8c9ccccc9c9ccccc9c8cc7c6c5)c4)n3)cc2)cc1]

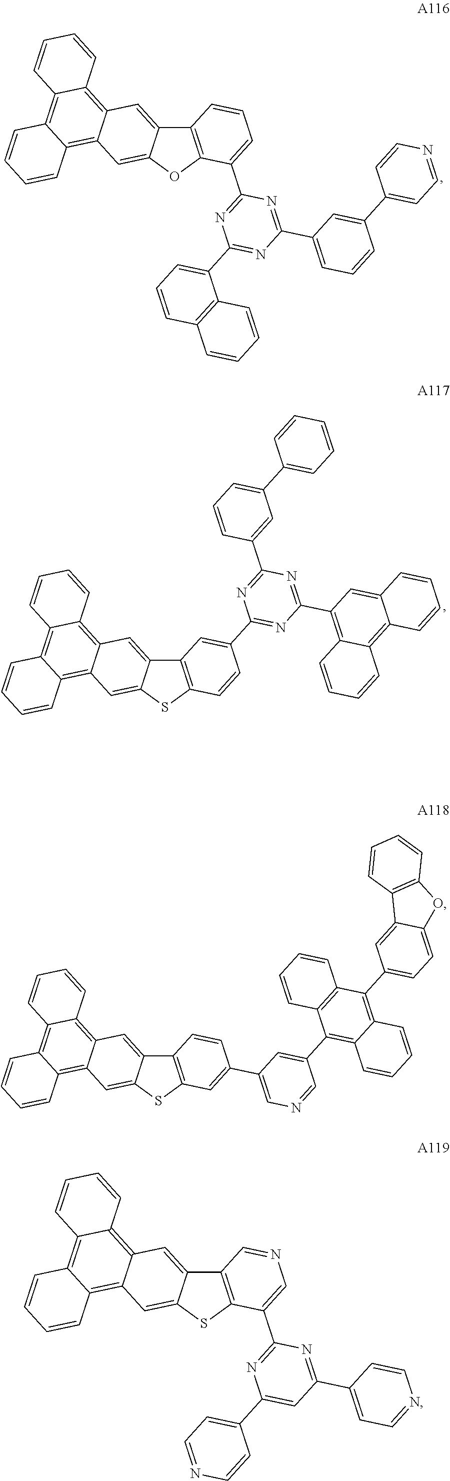 [SMILES: c1cc(-c2ccncc2)cc(-c2nc(-c3cccc4ccccc34)nc(-c3cccc4c3oc3cc5c6ccccc6c6ccccc6c5cc34)n2)c1 ; c1ccc(-c2cccc(-c3nc(-c4ccc5sc6cc7c8ccccc8c8ccccc8c7cc6c5c4)nc(-c4cc5ccccc5c5ccccc45)n3)c2)cc1 ; c1ccc2c(c1)oc1ccc(-c3c4ccccc4c(-c4cncc(-c5ccc6c(c5)sc5cc7c8ccccc8c8ccccc8c7cc56)c4)c4ccccc34)cc12 ; c1ccc2c(c1)c1ccccc1c1cc3c(cc21)sc1c(-c2nc(-c4ccncc4)cc(-c4ccncc4)n2)cncc13]

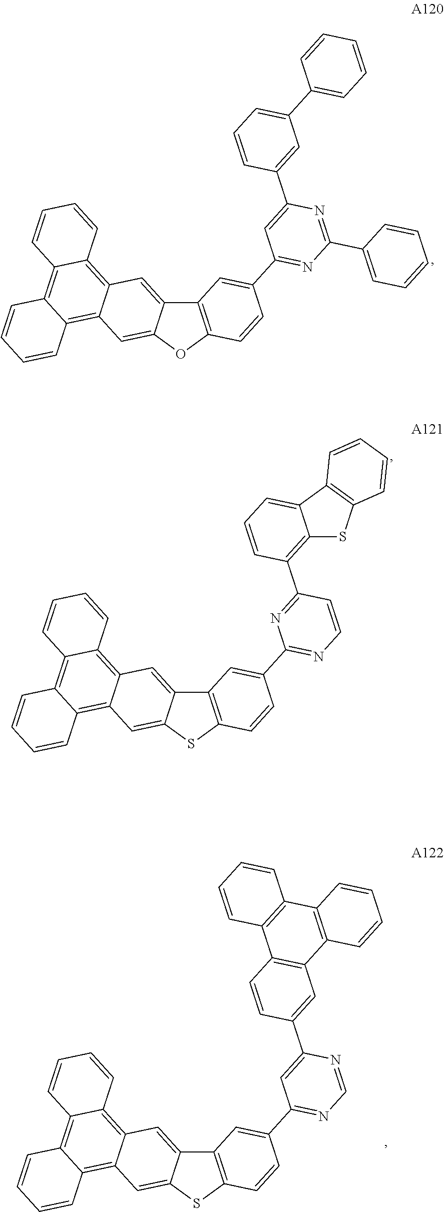 [SMILES: c1ccc(-c2cccc(-c3cc(-c4ccc5oc6cc7c8ccccc8c8ccccc8c7cc6c5c4)nc(-c4ccccc4)n3)c2)cc1 ; c1ccc2c(c1)sc1c(-c3ccnc(-c4ccc5sc6cc7c8ccccc8c8ccccc8c7cc6c5c4)n3)cccc12 ; c1ccc2c(c1)c1ccccc1c1cc(-c3cc(-c4ccc5sc6cc7c8ccccc8c8ccccc8c7cc6c5c4)ncn3)ccc21]

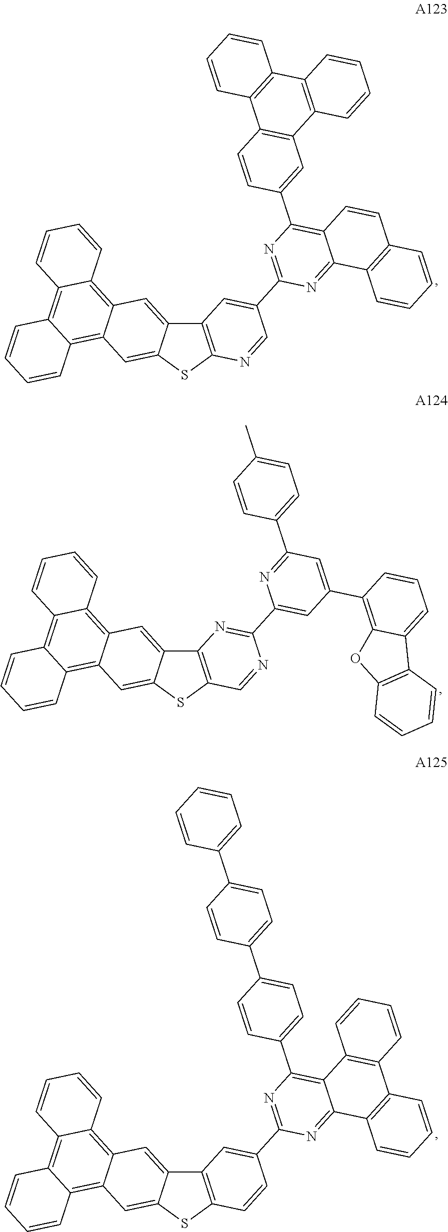 [SMILES: c1ccc2c(c1)ccc1c(-c3ccc4c5ccccc5c5ccccc5c4c3)nc(-c3cnc4sc5cc6c7ccccc7c7ccccc7c6cc5c4c3)nc12 ; Cc1ccc(-c2cc(-c3cccc4c3oc3ccccc34)cc(-c3ncc4sc5cc6c7ccccc7c7ccccc7c6cc5c4n3)n2)cc1 ; c1ccc(-c2ccc(-c3ccc(-c4nc(-c5ccc6sc7cc8c9ccccc9c9ccccc9c8cc7c6c5)nc5c6ccccc6c6ccccc6c45)cc3)cc2)cc1]

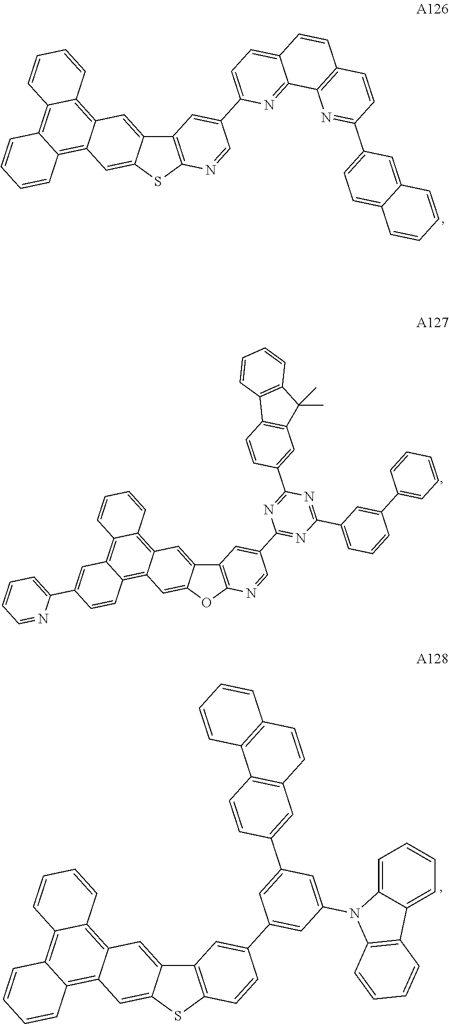 [SMILES: c1ccc2cc(-c3ccc4ccc5ccc(-c6cnc7sc8cc9c%10ccccc%10c%10ccccc%10c9cc8c7c6)nc5c4n3)ccc2c1 ; CC1(C)c2ccccc2-c2ccc(-c3nc(-c4cccc(-c5ccccc5)c4)nc(-c4cnc5oc6cc7c8ccc(-c9ccccn9)cc8c8ccccc8c7cc6c5c4)n3)cc21 ; c1ccc2c(c1)ccc1cc(-c3cc(-c4ccc5sc6cc7c8ccccc8c8ccccc8c7cc6c5c4)cc(-n4c5ccccc5c5ccccc54)c3)ccc12]

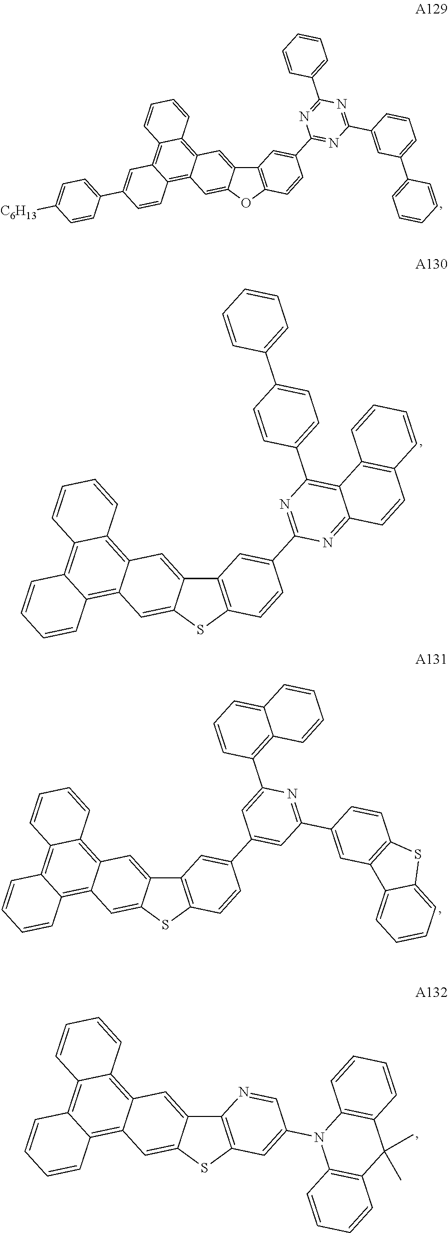

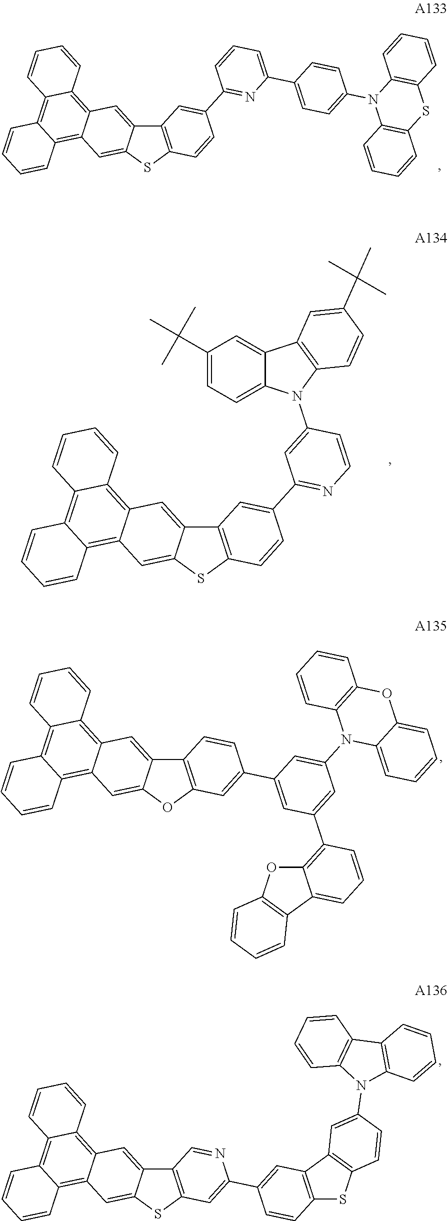 [SMILES: c1cc(-c2ccc(N3c4ccccc4Sc4ccccc43)cc2)nc(-c2ccc3sc4cc5c6ccccc6c6ccccc6c5cc4c3c2)c1 ; CC(C)(C)c1ccc2c(c1)c1cc(C(C)(C)C)ccc1n2-c1ccnc(-c2ccc3sc4cc5c6ccccc6c6ccccc6c5cc4c3c2)c1 ; c1ccc2c(c1)Oc1ccccc1N2c1cc(-c2ccc3c(c2)oc2cc4c5ccccc5c5ccccc5c4cc23)cc(-c2cccc3c2oc2ccccc23)c1 ; c1ccc2c(c1)c1ccccc1c1cc3c(cc21)sc1cc(-c2ccc4sc5ccc(-n6c7ccccc7c7ccccc76)cc5c4c2)ncc13]

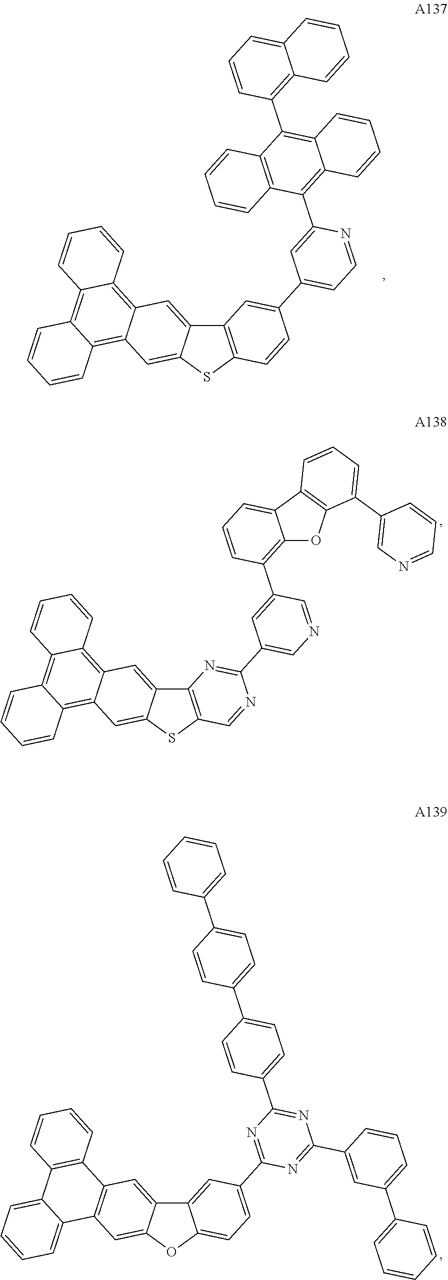 [SMILES: c1ccc2c(-c3c4ccccc4c(-c4cc(-c5ccc6sc7cc8c9ccccc9c9ccccc9c8cc7c6c5)ccn4)c4ccccc34)cccc2c1 ; c1cncc(-c2cccc3c2oc2c(-c4cncc(-c5ncc6sc7cc8c9ccccc9c9ccccc9c8cc7c6n5)c4)cccc23)c1 ; c1ccc(-c2ccc(-c3ccc(-c4nc(-c5cccc(-c6ccccc6)c5)nc(-c5ccc6oc7cc8c9ccccc9c9ccccc9c8cc7c6c5)n4)cc3)cc2)cc1]

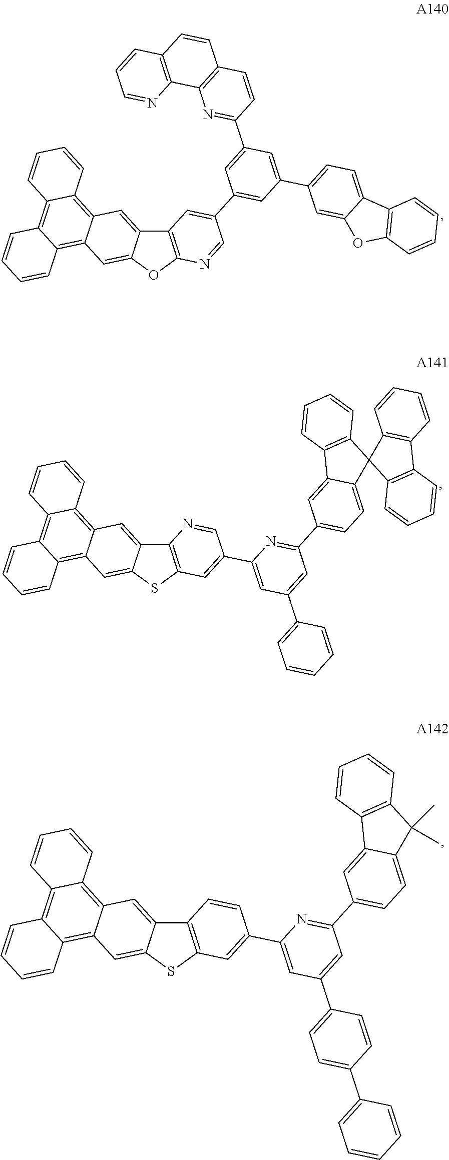 [SMILES: c1cnc2c(c1)ccc1ccc(-c3cc(-c4ccc5c(c4)oc4ccccc45)cc(-c4cnc5oc6cc7c8ccccc8c8ccccc8c7cc6c5c4)c3)nc12 ; c1ccc(-c2cc(-c3ccc4c(c3)-c3ccccc3C43c4ccccc4-c4ccccc43)nc(-c3cnc4c(c3)sc3cc5c6ccccc6c6ccccc6c5cc34)c2)cc1 ; CC1(C)c2ccccc2-c2cc(-c3cc(-c4ccc(-c5ccccc5)cc4)cc(-c4ccc5c(c4)sc4cc6c7ccccc7c7ccccc7c6cc45)n3)ccc21]

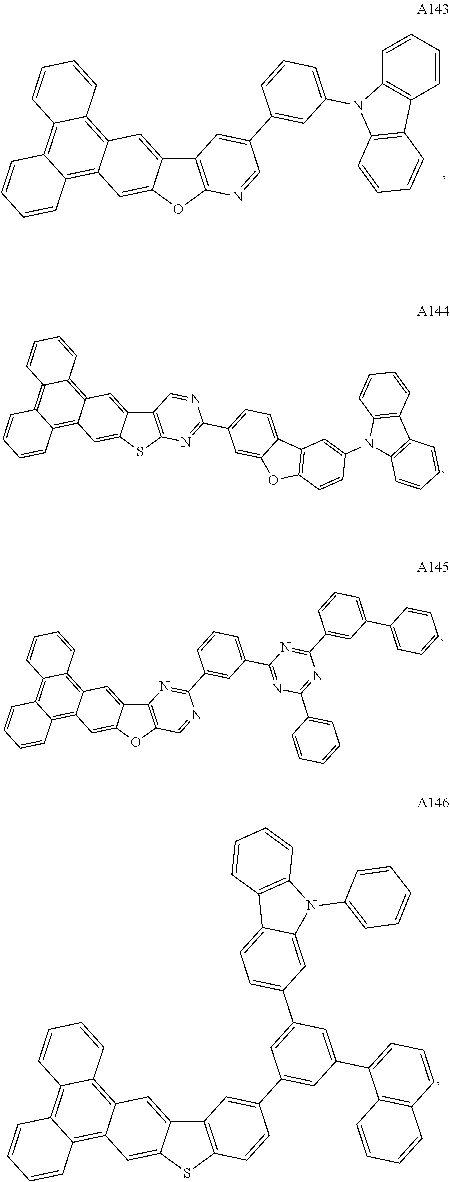 [SMILES: c1cc(-c2cnc3oc4cc5c6ccccc6c6ccccc6c5cc4c3c2)cc(-n2c3ccccc3c3ccccc32)c1 ; c1ccc2c(c1)c1ccccc1c1cc3c(cc21)sc1nc(-c2ccc4c(c2)oc2ccc(-n5c6ccccc6c6ccccc65)cc24)ncc13 ; c1ccc(-c2cccc(-c3nc(-c4ccccc4)nc(-c4cccc(-c5ncc6oc7cc8c9ccccc9c9ccccc9c8cc7c6n5)c4)n3)c2)cc1 ; c1ccc(-n2c3ccccc3c3ccc(-c4cc(-c5ccc6sc7cc8c9ccccc9c9ccccc9c8cc7c6c5)cc(-c5cccc6ccccc56)c4)cc32)cc1]

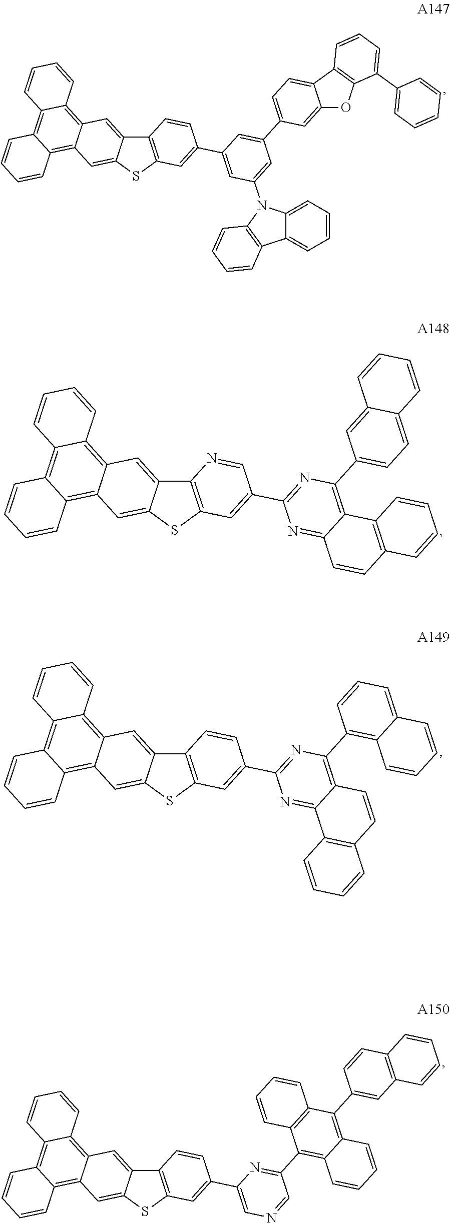 [SMILES: c1ccc(-c2cccc3c2oc2cc(-c4cc(-c5ccc6c(c5)sc5cc7c8ccccc8c8ccccc8c7cc56)cc(-n5c6ccccc6c6ccccc65)c4)ccc23)cc1 ; c1ccc2cc(-c3nc(-c4cnc5c(c4)sc4cc6c7ccccc7c7ccccc7c6cc45)nc4ccc5ccccc5c34)ccc2c1 ; c1ccc2c(-c3nc(-c4ccc5c(c4)sc4cc6c7ccccc7c7ccccc7c6cc45)nc4c3ccc3ccccc34)cccc2c1 ; c1ccc2cc(-c3c4ccccc4c(-c4cncc(-c5ccc6c(c5)sc5cc7c8ccccc8c8ccccc8c7cc56)n4)c4ccccc34)ccc2c1]

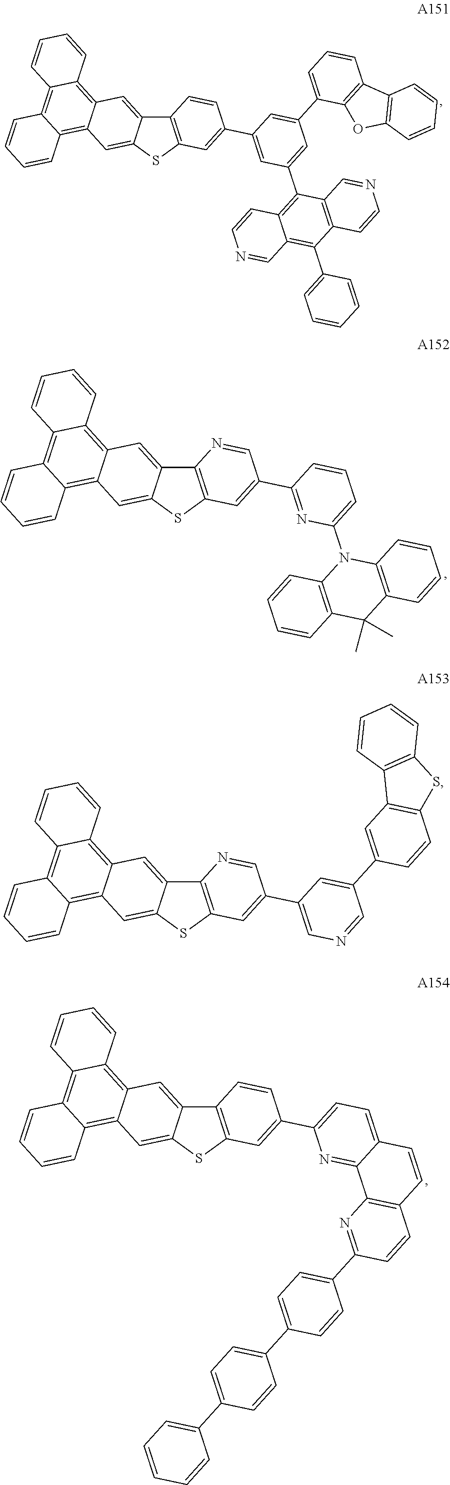 [SMILES: c1ccc(-c2c3ccncc3c(-c3cc(-c4ccc5c(c4)sc4cc6c7ccccc7c7ccccc7c6cc45)cc(-c4cccc5c4oc4ccccc45)c3)c3ccncc23)cc1 ; CC1(C)c2ccccc2N(c2cccc(-c3cnc4c(c3)sc3cc5c6ccccc6c6ccccc6c5cc34)n2)c2ccccc21 ; c1ccc2c(c1)sc1ccc(-c3cncc(-c4cnc5c(c4)sc4cc6c7ccccc7c7ccccc7c6cc45)c3)cc12 ; c1ccc(-c2ccc(-c3ccc(-c4ccc5ccc6ccc(-c7ccc8c(c7)sc7cc9c%10ccccc%10c%10ccccc%10c9cc78)nc6c5n4)cc3)cc2)cc1]

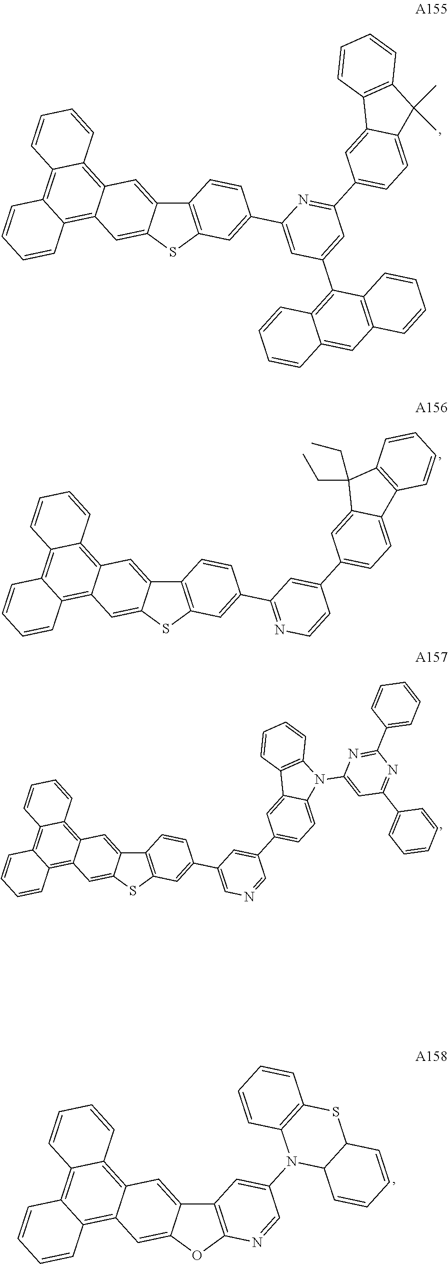 [SMILES: CC1(C)c2ccccc2-c2cc(-c3cc(-c4c5ccccc5cc5ccccc45)cc(-c4ccc5c(c4)sc4cc6c7ccccc7c7ccccc7c6cc45)n3)ccc21 ; CCC1(CC)c2ccccc2-c2ccc(-c3ccnc(-c4ccc5c(c4)sc4cc6c7ccccc7c7ccccc7c6cc45)c3)cc21 ; c1ccc(-c2cc(-n3c4ccccc4c4cc(-c5cncc(-c6ccc7c(c6)sc6cc8c9ccccc9c9ccccc9c8cc67)c5)ccc43)nc(-c3ccccc3)n2)cc1 ; C1=CC2Sc3ccccc3N(c3cnc4oc5cc6c7ccccc7c7ccccc7c6cc5c4c3)C2C=C1]

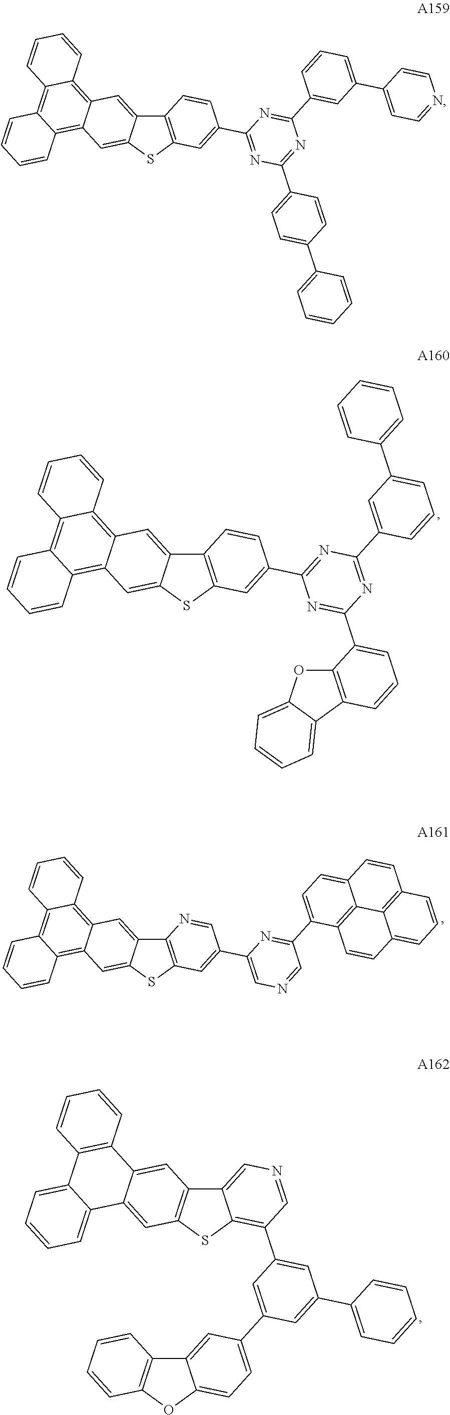 [SMILES: c1ccc(-c2ccc(-c3nc(-c4cccc(-c5ccncc5)c4)nc(-c4ccc5c(c4)sc4cc6c7ccccc7c7ccccc7c6cc45)n3)cc2)cc1 ; c1ccc(-c2cccc(-c3nc(-c4ccc5c(c4)sc4cc6c7ccccc7c7ccccc7c6cc45)nc(-c4cccc5c4oc4ccccc45)n3)c2)cc1 ; c1cc2ccc3ccc(-c4cncc(-c5cnc6c(c5)sc5cc7c8ccccc8c8ccccc8c7cc56)n4)c4ccc(c1)c2c34 ; c1ccc(-c2cc(-c3ccc4oc5ccccc5c4c3)cc(-c3cncc4c3sc3cc5c6ccccc6c6ccccc6c5cc34)c2)cc1]

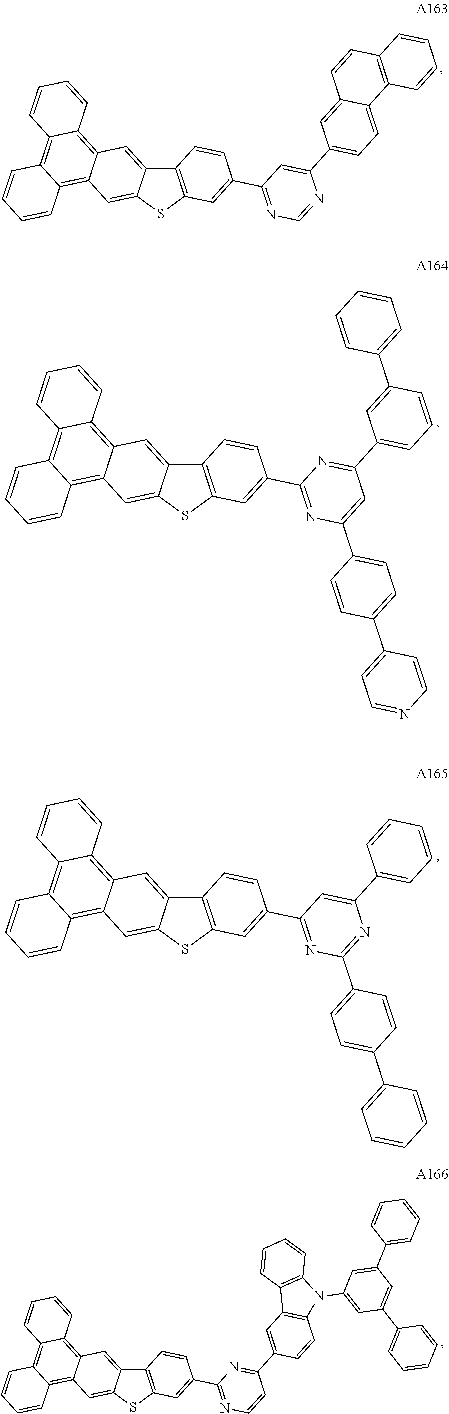 [SMILES: c1ccc2c(c1)ccc1cc(-c3cc(-c4ccc5c(c4)sc4cc6c7ccccc7c7ccccc7c6cc45)ncn3)ccc12 ; c1ccc(-c2cccc(-c3cc(-c4ccc(-c5ccncc5)cc4)nc(-c4ccc5c(c4)sc4cc6c7ccccc7c7ccccc7c6cc45)n3)c2)cc1 ; c1ccc(-c2ccc(-c3nc(-c4ccccc4)cc(-c4ccc5c(c4)sc4cc6c7ccccc7c7ccccc7c6cc45)n3)cc2)cc1 ; c1ccc(-c2cc(-c3ccccc3)cc(-n3c4ccccc4c4cc(-c5ccnc(-c6ccc7c(c6)sc6cc8c9ccccc9c9ccccc9c8cc67)n5)ccc43)c2)cc1]

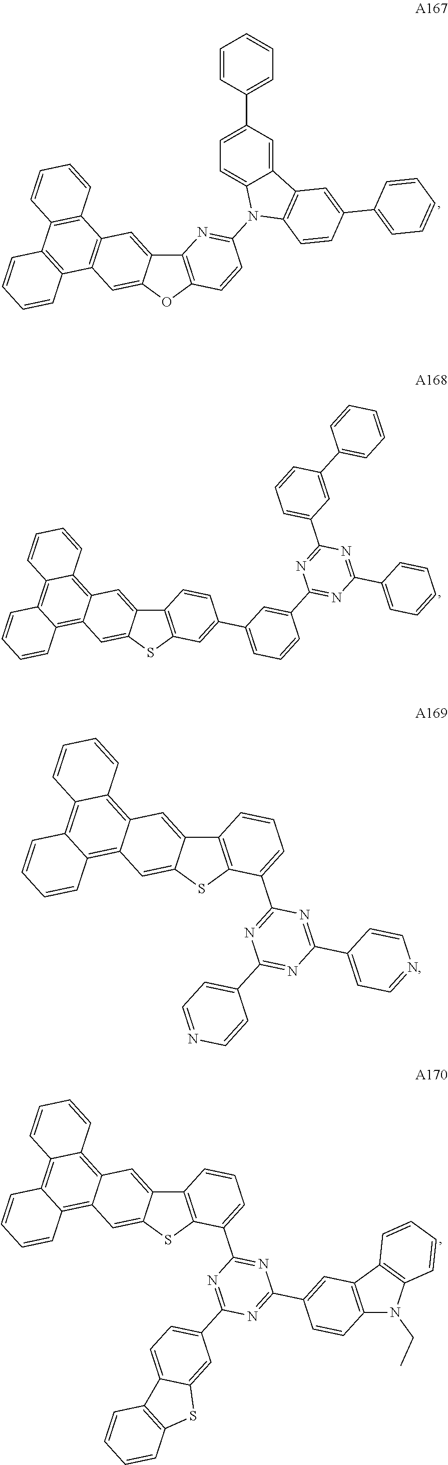 [SMILES: c1ccc(-c2ccc3c(c2)c2cc(-c4ccccc4)ccc2n3-c2ccc3oc4cc5c6ccccc6c6ccccc6c5cc4c3n2)cc1 ; c1ccc(-c2cccc(-c3nc(-c4ccccc4)nc(-c4cccc(-c5ccc6c(c5)sc5cc7c8ccccc8c8ccccc8c7cc56)c4)n3)c2)cc1 ; c1cc(-c2nc(-c3ccncc3)nc(-c3ccncc3)n2)c2sc3cc4c5ccccc5c5ccccc5c4cc3c2c1 ; CCn1c2ccccc2c2cc(-c3nc(-c4ccc5c(c4)sc4ccccc45)nc(-c4cccc5c4sc4cc6c7ccccc7c7ccccc7c6cc45)n3)ccc21]

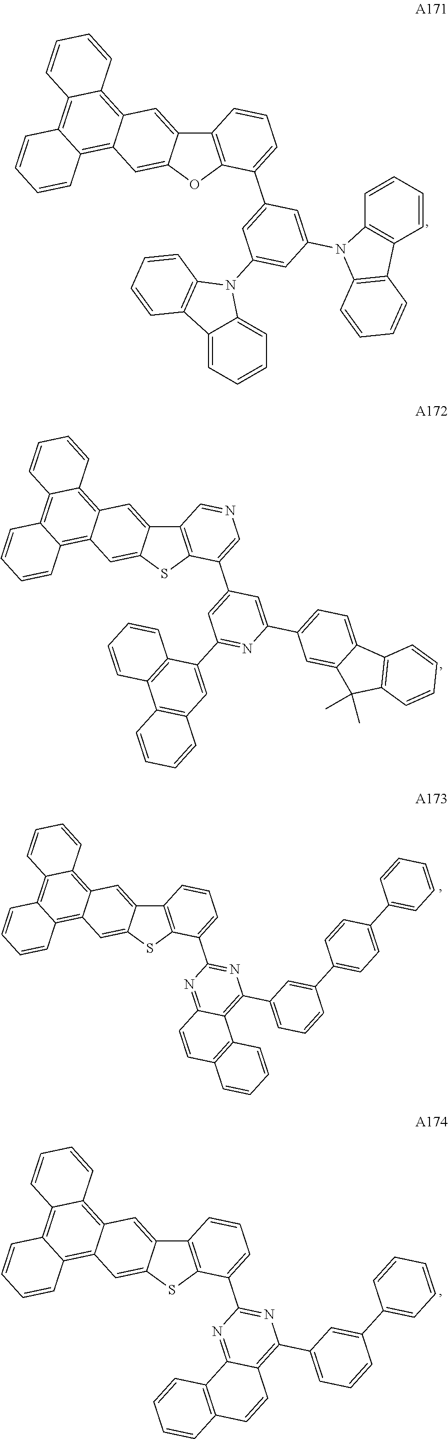 [SMILES: c1cc(-c2cc(-n3c4ccccc4c4ccccc43)cc(-n3c4ccccc4c4ccccc43)c2)c2oc3cc4c5ccccc5c5ccccc5c4cc3c2c1 ; CC1(C)c2ccccc2-c2ccc(-c3cc(-c4cncc5c4sc4cc6c7ccccc7c7ccccc7c6cc45)cc(-c4cc5ccccc5c5ccccc45)n3)cc21 ; c1ccc(-c2ccc(-c3cccc(-c4nc(-c5cccc6c5sc5cc7c8ccccc8c8ccccc8c7cc56)nc5ccc6ccccc6c45)c3)cc2)cc1 ; c1ccc(-c2cccc(-c3nc(-c4cccc5c4sc4cc6c7ccccc7c7ccccc7c6cc45)nc4c3ccc3ccccc34)c2)cc1]

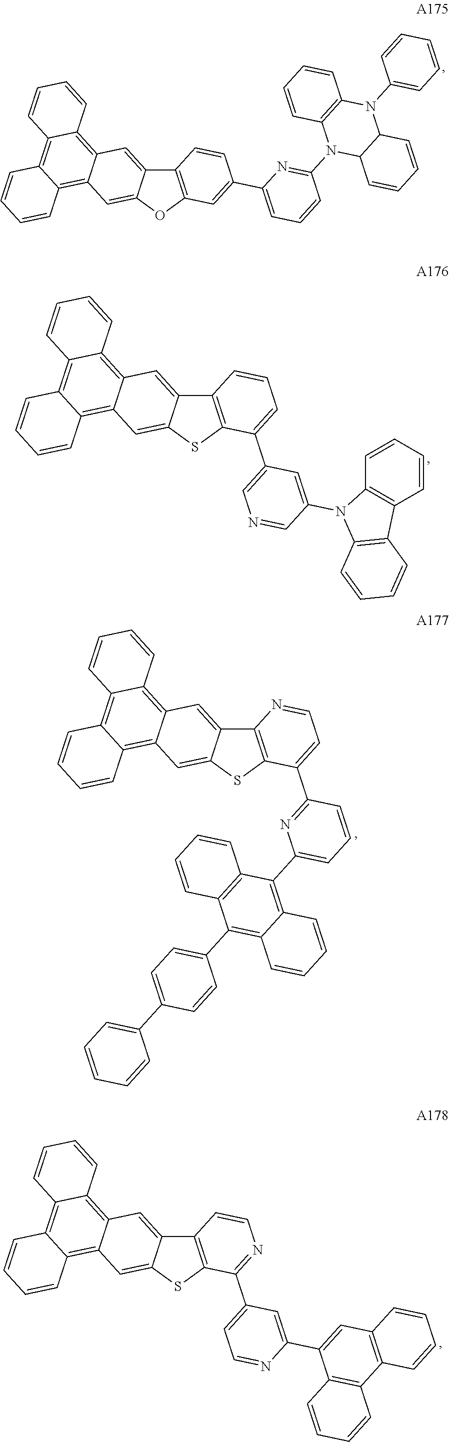 [SMILES: C1=CC2C(C=C1)N(c1cccc(-c3ccc4c(c3)oc3cc5c6ccccc6c6ccccc6c5cc34)n1)c1ccccc1N2c1ccccc1 ; c1cc(-c2cncc(-n3c4ccccc4c4ccccc43)c2)c2sc3cc4c5ccccc5c5ccccc5c4cc3c2c1 ; c1ccc(-c2ccc(-c3c4ccccc4c(-c4cccc(-c5ccnc6c5sc5cc7c8ccccc8c8ccccc8c7cc56)n4)c4ccccc34)cc2)cc1 ; c1ccc2c(c1)cc(-c1cc(-c3nccc4c3sc3cc5c6ccccc6c6ccccc6c5cc34)ccn1)c1ccccc12]

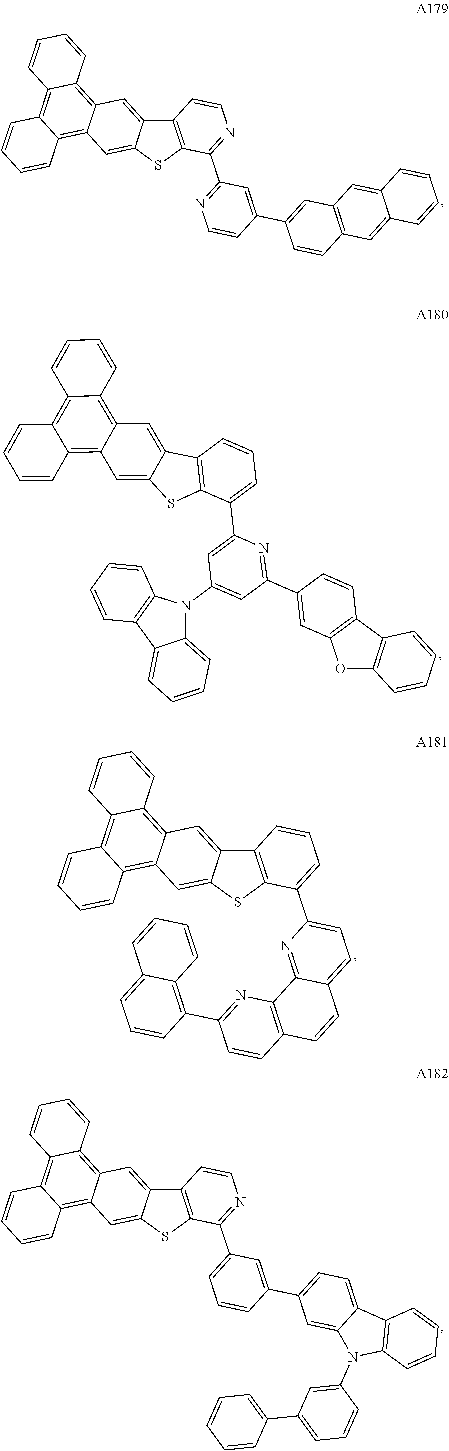 [SMILES: c1ccc2cc3cc(-c4ccnc(-c5nccc6c5sc5cc7c8ccccc8c8ccccc8c7cc56)c4)ccc3cc2c1 ; c1ccc2c(c1)oc1cc(-c3cc(-n4c5ccccc5c5ccccc54)cc(-c4cccc5c4sc4cc6c7ccccc7c7ccccc7c6cc45)n3)ccc12 ; c1ccc2c(-c3ccc4ccc5ccc(-c6cccc7c6sc6cc8c9ccccc9c9ccccc9c8cc67)nc5c4n3)cccc2c1 ; c1ccc(-c2cccc(-n3c4ccccc4c4ccc(-c5cccc(-c6nccc7c6sc6cc8c9ccccc9c9ccccc9c8cc67)c5)cc43)c2)cc1]

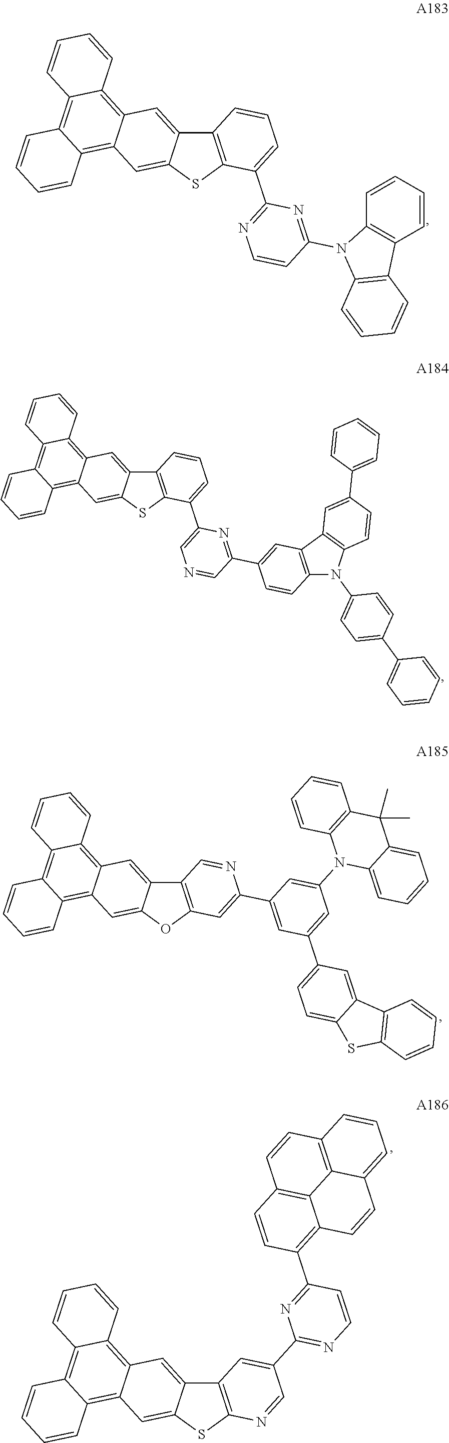 [SMILES: c1cc(-c2nccc(-n3c4ccccc4c4ccccc43)n2)c2sc3cc4c5ccccc5c5ccccc5c4cc3c2c1 ; c1ccc(-c2ccc(-n3c4ccc(-c5ccccc5)cc4c4cc(-c5cncc(-c6cccc7c6sc6cc8c9ccccc9c9ccccc9c8cc67)n5)ccc43)cc2)cc1 ; CC1(C)c2ccccc2N(c2cc(-c3ccc4sc5ccccc5c4c3)cc(-c3cc4oc5cc6c7ccccc7c7ccccc7c6cc5c4cn3)c2)c2ccccc21 ; c1cc2ccc3ccc(-c4ccnc(-c5cnc6sc7cc8c9ccccc9c9ccccc9c8cc7c6c5)n4)c4ccc(c1)c2c34]

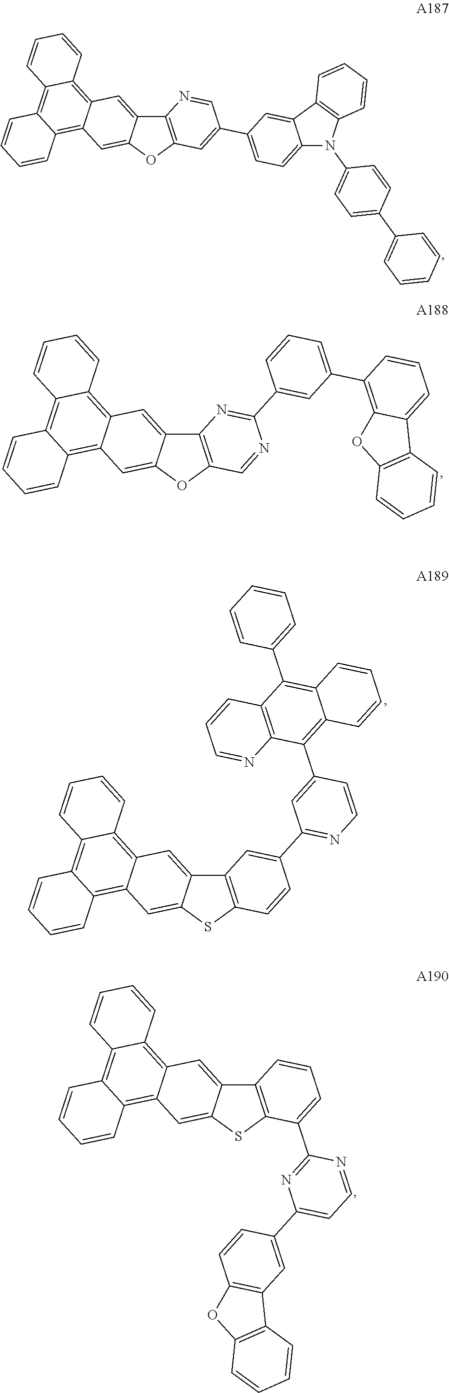 [SMILES: c1ccc(-c2ccc(-n3c4ccccc4c4cc(-c5cnc6c(c5)oc5cc7c8ccccc8c8ccccc8c7cc56)ccc43)cc2)cc1 ; c1cc(-c2ncc3oc4cc5c6ccccc6c6ccccc6c5cc4c3n2)cc(-c2cccc3c2oc2ccccc23)c1 ; c1ccc(-c2c3ccccc3c(-c3ccnc(-c4ccc5sc6cc7c8ccccc8c8ccccc8c7cc6c5c4)c3)c3ncccc23)cc1 ; c1ccc2c(c1)oc1ccc(-c3ccnc(-c4cccc5c4sc4cc6c7ccccc7c7ccccc7c6cc45)n3)cc12]

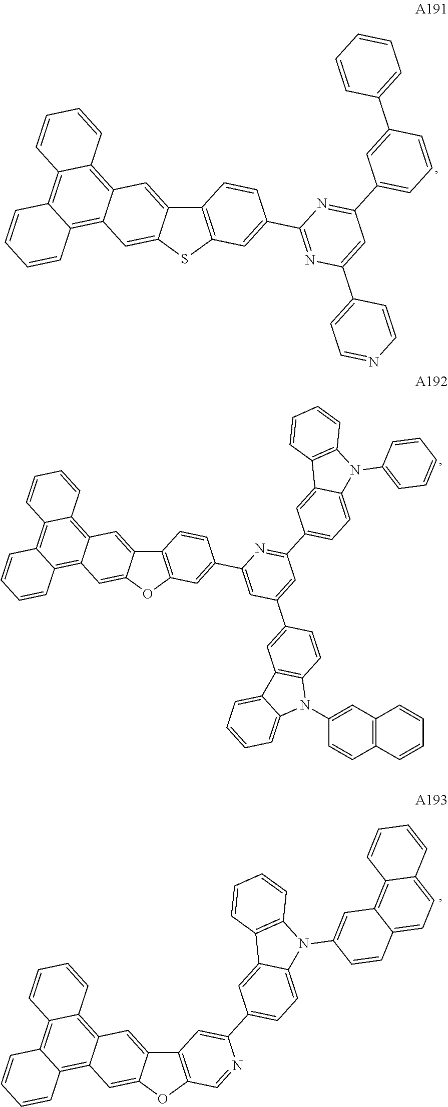 [SMILES: c1ccc(-c2cccc(-c3cc(-c4ccncc4)nc(-c4ccc5c(c4)sc4cc6c7ccccc7c7ccccc7c6cc45)n3)c2)cc1 ; c1ccc(-n2c3ccccc3c3cc(-c4cc(-c5ccc6c(c5)c5ccccc5n6-c5ccc6ccccc6c5)cc(-c5ccc6c(c5)oc5cc7c8ccccc8c8ccccc8c7cc56)n4)ccc32)cc1 ; c1ccc2c(c1)ccc1ccc(-n3c4ccccc4c4cc(-c5cc6c(cn5)oc5cc7c8ccccc8c8ccccc8c7cc56)ccc43)cc12]

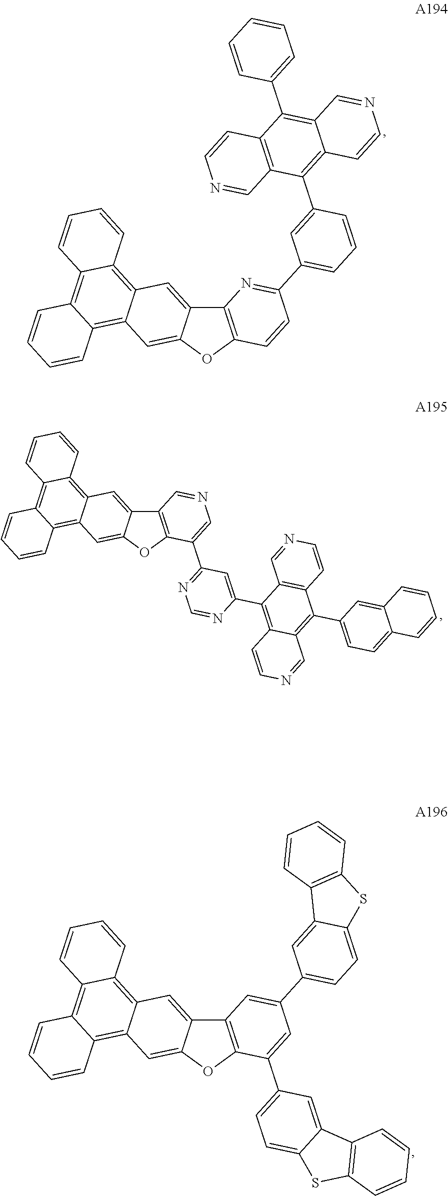 [SMILES: c1ccc(-c2c3ccncc3c(-c3cccc(-c4ccc5oc6cc7c8ccccc8c8ccccc8c7cc6c5n4)c3)c3ccncc23)cc1 ; c1ccc2cc(-c3c4cnccc4c(-c4cc(-c5cncc6c5oc5cc7c8ccccc8c8ccccc8c7cc56)ncn4)c4cnccc34)ccc2c1 ; c1ccc2c(c1)sc1ccc(-c3cc(-c4ccc5sc6ccccc6c5c4)c4oc5cc6c7ccccc7c7ccccc7c6cc5c4c3)cc12]

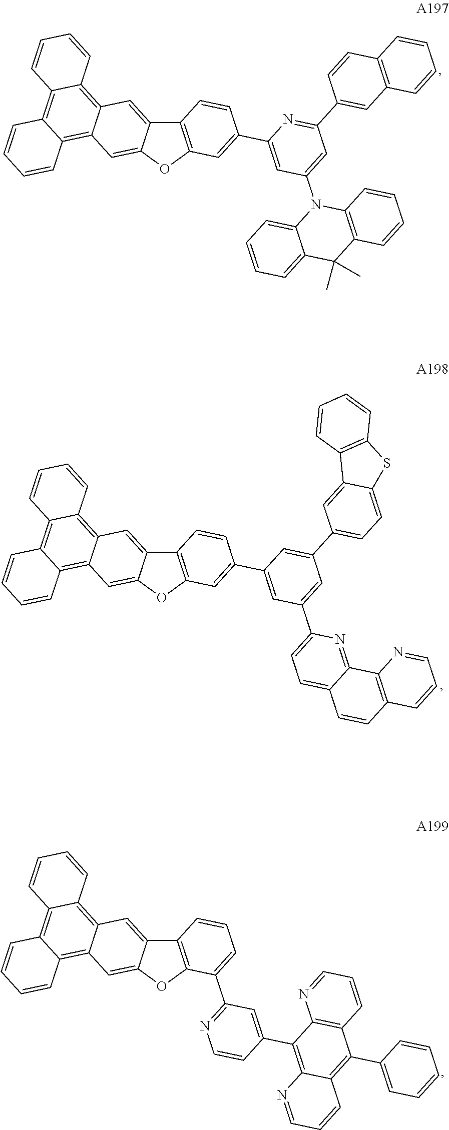 [SMILES: CC1(C)c2ccccc2N(c2cc(-c3ccc4ccccc4c3)nc(-c3ccc4c(c3)oc3cc5c6ccccc6c6ccccc6c5cc34)c2)c2ccccc21 ; c1cnc2c(c1)ccc1ccc(-c3cc(-c4ccc5c(c4)oc4cc6c7ccccc7c7ccccc7c6cc45)cc(-c4ccc5sc6ccccc6c5c4)c3)nc12 ; c1ccc(-c2c3cccnc3c(-c3ccnc(-c4cccc5c4oc4cc6c7ccccc7c7ccccc7c6cc45)c3)c3ncccc23)cc1]

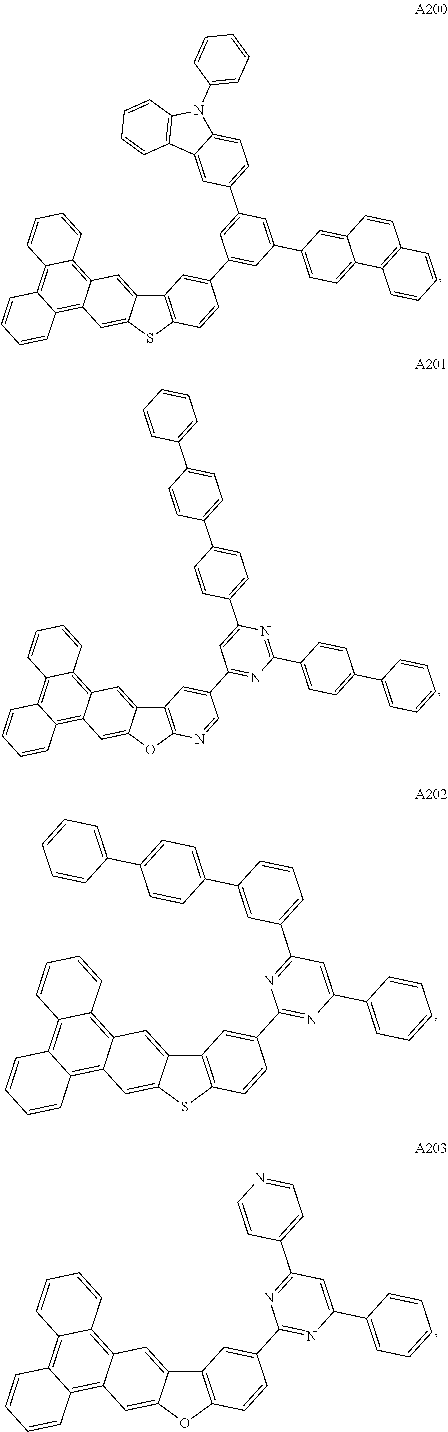 [SMILES: c1ccc(-n2c3ccccc3c3cc(-c4cc(-c5ccc6c(ccc7ccccc76)c5)cc(-c5ccc6sc7cc8c9ccccc9c9ccccc9c8cc7c6c5)c4)ccc32)cc1 ; c1ccc(-c2ccc(-c3ccc(-c4cc(-c5cnc6oc7cc8c9ccccc9c9ccccc9c8cc7c6c5)nc(-c5ccc(-c6ccccc6)cc5)n4)cc3)cc2)cc1 ; c1ccc(-c2ccc(-c3cccc(-c4cc(-c5ccccc5)nc(-c5ccc6sc7cc8c9ccccc9c9ccccc9c8cc7c6c5)n4)c3)cc2)cc1 ; c1ccc(-c2cc(-c3ccncc3)nc(-c3ccc4oc5cc6c7ccccc7c7ccccc7c6cc5c4c3)n2)cc1]

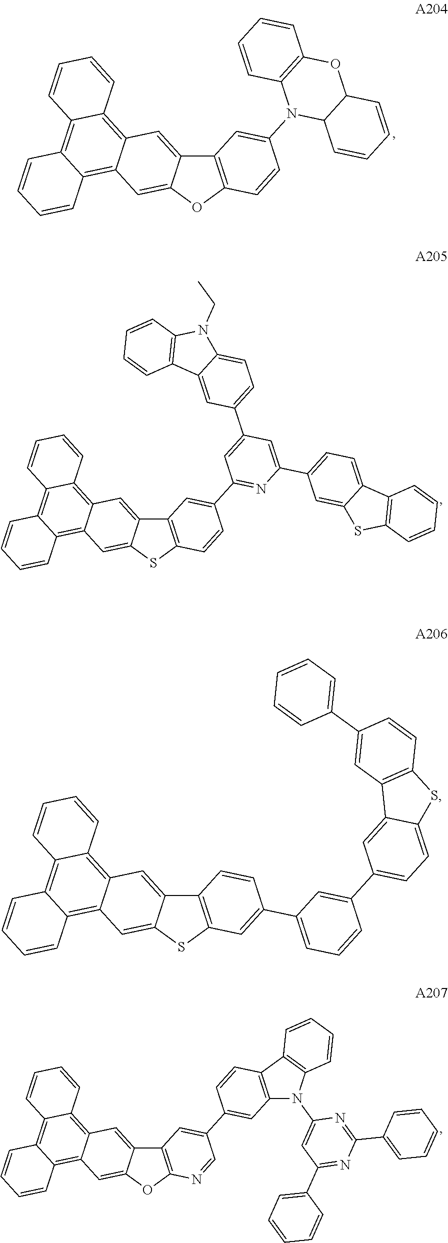 [SMILES: C1=CC2Oc3ccccc3N(c3ccc4oc5cc6c7ccccc7c7ccccc7c6cc5c4c3)C2C=C1 ; CCn1c2ccccc2c2cc(-c3cc(-c4ccc5c(c4)sc4ccccc45)nc(-c4ccc5sc6cc7c8ccccc8c8ccccc8c7cc6c5c4)c3)ccc21 ; c1ccc(-c2ccc3sc4ccc(-c5cccc(-c6ccc7c(c6)sc6cc8c9ccccc9c9ccccc9c8cc67)c5)cc4c3c2)cc1 ; c1ccc(-c2cc(-n3c4ccccc4c4ccc(-c5cnc6oc7cc8c9ccccc9c9ccccc9c8cc7c6c5)cc43)nc(-c3ccccc3)n2)cc1]

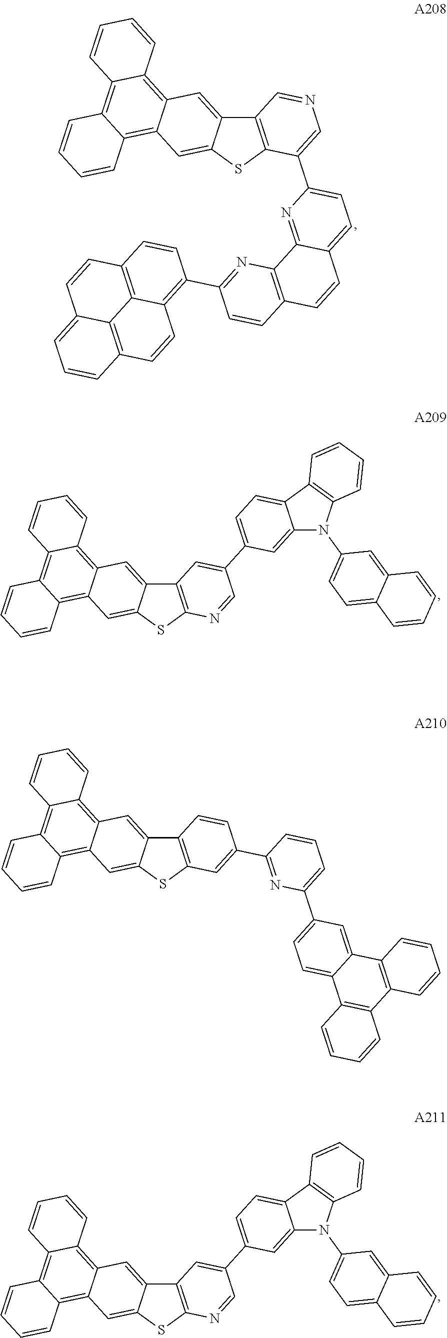 [SMILES: c1cc2ccc3ccc(-c4ccc5ccc6ccc(-c7cncc8c7sc7cc9c%10ccccc%10c%10ccccc%10c9cc78)nc6c5n4)c4ccc(c1)c2c34 ; c1ccc2cc(-n3c4ccccc4c4ccc(-c5cnc6sc7cc8c9ccccc9c9ccccc9c8cc7c6c5)cc43)ccc2c1 ; c1cc(-c2ccc3c(c2)sc2cc4c5ccccc5c5ccccc5c4cc23)nc(-c2ccc3c4ccccc4c4ccccc4c3c2)c1 ; c1ccc2cc(-n3c4ccccc4c4ccc(-c5cnc6sc7cc8c9ccccc9c9ccccc9c8cc7c6c5)cc43)ccc2c1]

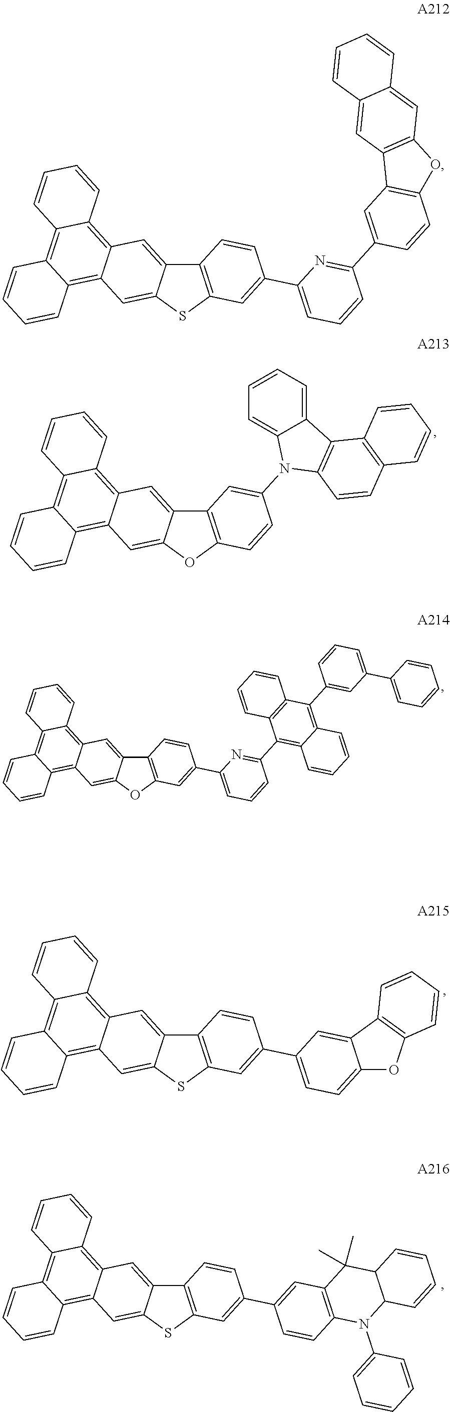 [SMILES: c1cc(-c2ccc3c(c2)sc2cc4c5ccccc5c5ccccc5c4cc23)nc(-c2ccc3oc4cc5ccccc5cc4c3c2)c1 ; c1ccc2c(c1)ccc1c2c2ccccc2n1-c1ccc2oc3cc4c5ccccc5c5ccccc5c4cc3c2c1 ; c1ccc(-c2cccc(-c3c4ccccc4c(-c4cccc(-c5ccc6c(c5)oc5cc7c8ccccc8c8ccccc8c7cc56)n4)c4ccccc34)c2)cc1 ; c1ccc2c(c1)oc1ccc(-c3ccc4c(c3)sc3cc5c6ccccc6c6ccccc6c5cc34)cc12 ; CC1(C)c2cc(-c3ccc4c(c3)sc3cc5c6ccccc6c6ccccc6c5cc34)ccc2N(c2ccccc2)C2C=CC=CC21]

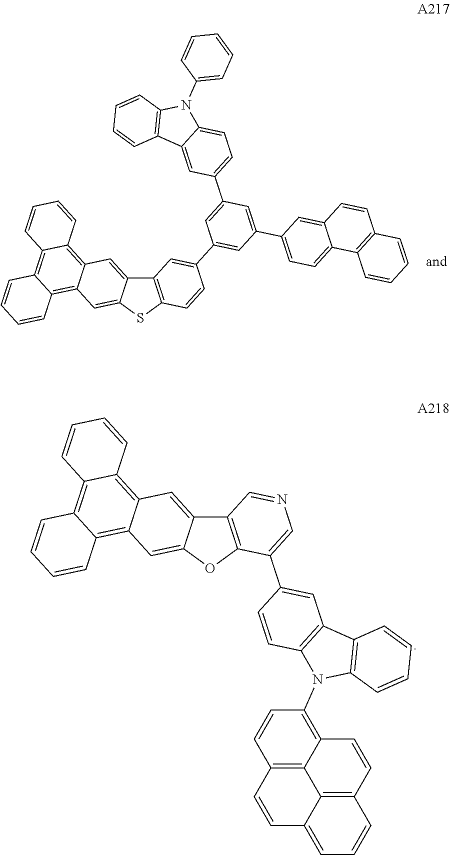 [SMILES: c1ccc(-n2c3ccccc3c3cc(-c4cc(-c5ccc6c(ccc7ccccc76)c5)cc(-c5ccc6sc7cc8c9ccccc9c9ccccc9c8cc7c6c5)c4)ccc32)cc1 ; c1cc2ccc3ccc(-n4c5ccccc5c5cc(-c6cncc7c6oc6cc8c9ccccc9c9ccccc9c8cc67)ccc54)c4ccc(c1)c2c34]

6. An organic electroluminescence device comprising a pair of electrodes having a cathode and an anode, and between the pairs of electrodes comprising at least a light emitting layer and one or more layers of organic thin film layers, wherein the light emitting layer and/or the one or more thin film layers comprise the organic compound according to claim 5.

7. The organic electroluminescence device according to claim 6, wherein the light emitting layer comprising the organic compound of formula (A) is a host material.

8. The organic electroluminescent device according to claim 6, wherein the one or more layers of organic thin film layers comprises an electron transport layer having an organic material of formula (A).

9. The organic electroluminescent device according to claim 6, wherein the one or more layers of organic thin film layers comprises a hole blocking layer having an organic material of formula (A).

9. The organic electroluminescence device according to claim 6, wherein the light emitting layer comprising the organic compound of formula (A) is a fluorescent emitter.

10. The organic electroluminescence device according to claim 6, wherein the light emitting layer emits fluorescent blue lights.

11. The organic electroluminescent device according to claim 6, wherein the device is a lighting panel.

12. The organic electroluminescent device according to claim 6, wherein the device is a backlight panel.
